(19) United States
(12) Patent Application Publication
(10) Pub. No.: US 2016/0166560 A1
(43) Pub. Date: Jun. 16, 2016

(54) Urea Compounds and Their Use as FAAH Enzyme Inhibitors

(71) Applicant: BIAL-PORTELA & Cª, S.A., Coronado (PT)

(72) Inventors: Laszlo Erno Kiss, S. Mamede do Coronado (PT); Rita Gusmão De Noronha, S. Mamede do Coronado (PT); Carla Patrícia Da Costa P Rosa, S. Mamede do Coronado (PT); Rui Pinto, S. Mamede do Coronado (PT)

(21) Appl. No.: 14/908,696

(22) PCT Filed: Aug. 1, 2014
(86) PCT No.: PCT/PT14/00051

§ 371 Date: Jan. 29, 2016

(30) Foreign Application Priority Data
Aug. 1, 2013 (GB) 1313786.4
May 28, 2014 (GB) 1409466.8

(51) Int. Cl. A61K 31/454; C07D 405/14; C07D 401/12; A61K 9/00; A61K 45/06

(52) U.S. Cl. CPC A61K 31/454; A61K 9/0014; A61K 45/06; C07D 401/12; C07D 405/14

(57) ABSTRACT

A compound having Formula I:

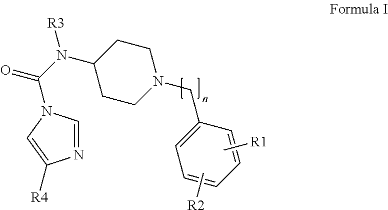

wherein: R1 is selected from hydrogen, halogen, hydroxyl and C₁₋₄ alkoxy; R2 is selected from hydrogen, halogen, hydroxyl and C₁₋₄ alkoxy; R3 is C₁₋₄ alkyl; R4 is aryl which is substituted with a group selected from OSO₂NH₂, NHCONH₂, NHSO₂NH₂, NHSO₂C₁₋₄ alkyl and CONH₂; and n is 0 or 1; or a pharmaceutically acceptable salt thereof; provided that the compound is not N-(1-benzylpiperidin-4-yl)-N-methyl-4-(4-(sulfamoylamino)phenyl)-1H-imidazole-1-carboxamide or N-(1-benzylpiperidin-4-yl)-N-methyl-4-(3-(methyl sulfonamido)phenyl)-1H-imidazole-1-carboxamide. The compound may be used as an inhibitor of fatty acid amide hydrolase.

### FIELD OF THE INVENTION

The present invention relates to water soluble urea compounds and their use. These compounds have been found to be useful in the treatment or prevention of conditions having an association with substrates, such as the neurotransmitter anandamide, which are broken down by the fatty acid amide hydrolase (FAAH) enzyme. In particular, due to their water soluble nature, it has been found that the compounds may be useful in the treatment or prevention of ocular conditions, such as ocular hypertension, ocular pain, dry eye syndrome, retinopathy, glaucoma and certain ocular inflammatory disorders such as uveitis, scleritis, episcleritis, episclera, keratitis, retinal vasculitis and chronic conjunctivitis. Furthermore, systemic administration may prove beneficial in more generalized conditions such as reduction of L-dopa-induced hyperactivity in adjunctive dopamine replacement therapy, bladder control and stress-related neuroinflammatory disorders such as post-traumatic stress disorder, multiple sclerosis and stroke.

### BACKGROUND TO THE INVENTION

FAAH enzyme breaks down fatty acid amides such as anandamide (N-arachidonoylethanolamine), N-oleoylethanolamine, N-palmitoylethanolamine and oleamide. Anandamide, also known as N-arachidonoylethanolamine or AEA, is an endogenous cannabinoid neurotransmitter found in animal and human organs, especially in the brain. It has also been found that anandamide binds to the vanilloid receptor. Anandamide is degraded by the fatty acid amide hydrolase (FAAH) enzyme to ethanolamine and arachidonic acid. Accordingly, inhibitors of FAAH lead to elevated anandamide levels.

Anandamide is a neurotransmitter in the endocannabinoid system and stimulates the cannabinoid receptors. Cannabinoid receptors, such as CB1 and CB2, are G protein-coupled receptors. CB1 is found mainly in the central nervous system whereas CB2 is found mainly in peripheral tissue. The endocannabinoid system has been implicated in a growing number of physiological functions, both in the central and peripheral nervous systems and in peripheral organs. Modulation of the activity of the endocannabinoid system has been shown to have a potentially therapeutic effect on a wide range of disparate diseases and pathological conditions. Therefore, the endocannabinoid system, and the FAAH enzyme in particular, has become a therapeutic target for developing potential treatments for many diseases. The endocannabinoid system has been implicated in appetite regulation, obesity, metabolic disorders, cachexia, anorexia, pain, inflammation, neurotoxicity, neurotrauma, stroke, multiple sclerosis, spinal cord injury, Parkinson's disease, levodopa-induced dyskinesia, Huntington's disease, Gilles de la Tourette's syndrome, tardive dyskinesia, dystonia, amyotrophic lateral sclerosis, Alzheimer's disease, epilepsy, schizophrenia, anxiety, depression, insomnia, nausea, emesis, alcohol disorders, drug addictions such as opiates, nicotine, cocaine, alcohol and psychostimulants, hypertension, circulatory shock, myocardial reperfusion injury, atherosclerosis, asthma, glaucoma, retinopathy, cancer, inflammatory bowel disease, acute and chronic liver disease such as hepatitis and liver cirrhosis, arthritis and osteoporosis. The endocannabinoid system and the conditions with which it is associated is discussed in detail in Pacher et al. Pharmacol. Rev. 2006, 58, 389-462.

In order to modulate the level of endogenous FAAH substrates, such as anandamide, which in turn modulate the endocannabinoid system, inhibitors of the FAAH enzyme have been developed. This allows conditions and diseases associated with the endocannabinoid system to be at least partially treated or prevented.

Since the substrates of FAAH bind to other receptors, e.g. the vanilloid receptor, and/or are involved in other signalling pathways, inhibitors of FAAH may also allow conditions or diseases associated with other pathways or systems, e.g. the vanilloid system, to be at least partially treated or prevented.

WO 2010/074588 discloses compounds which are inhibitors of FAAH.

Käsnäinen et al. (Heikki Käsnänen, Mikko J. Myllymäki, Anna Minkkili, Antti O. Kataja, Susanna M. Saario, Tapio Nevalainen, Ari M. P. Koskinen, and Antti Poso. Chem Med Chem 2010, 5(2), 213-231) discloses carbamate compounds which are FAAH inhibitors. In particular, compound 6b is a FAAH inhibitor which contains an imidazole structure. However, this compound is a weak FAAH inhibitor compared to many of the other carbamate compounds described in this paper and which do not contain an imidazole structure.

### SUMMARY OF THE INVENTION

In a first aspect, the present invention provides a compound having Formula I:

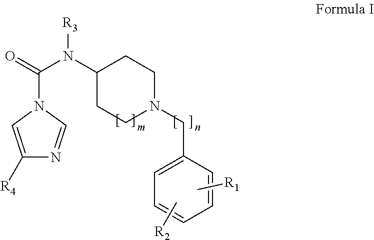

wherein:
R1 is selected from hydrogen, halogen, hydroxyl and C₁₋₄ alkoxy;
R2 is selected from hydrogen, halogen, hydroxyl and C₁₋₄ alkoxy;
R3 is C₁₋₄ alkyl;
R4 is aryl which is substituted with a group selected from OSO₂NH₂, NHCONH₂, NHSO₂NH₂, NHSO₂C₁₋₄ alkyl and CONH₂;
m is 0 or 1; and
n is 0 or 1;
or a pharmaceutically acceptable salt thereof;
provided that the compound is not N-(1-benzylpiperidin-4-yl)-N-methyl-4-(4-(sulfamoylamino)phenyl)-1H-imidazole-1-carboxamide or N-(1-benzylpiperidin-4-yl)-N-methyl-4-(3-(methylsulfonamido)phenyl)-1H-imidazole-1-carboxamide.

The compounds of the invention have been found to modulate the activity of the enzyme fatty acid amide hydrolase (FAAH). These compounds are relatively water soluble and have been found to be useful in the treatment of conditions for which water soluble drugs could be advantageously used. The compounds can be administered topically or systemically due to their water solubility. The compounds possess one or more of the following properties that make them particularly suitable for certain conditions: they are peripherally selective so that they inhibit FAAH to a greater extent in peripheral tissue compared to central nervous system tissue; they are relatively potent and they are relatively water soluble. Further, many of these compounds also display other advantages. For example, it is thought that the genotoxicity risk of at least some of these compounds is reduced so that it is relatively low. Further, these compounds have been found to be particularly useful in the treatment of ocular conditions. For ocular conditions, the compounds are able to penetrate the eye, for example, by crossing the blood-ocular barrier (FAAH is a potential therapeutic target for the treatment of eye diseases (see Pacher et al. Pharmacol. Rev. 2006, 58, 389-462 and Nucci et al., Investigative Ophthalmology & Visual Science, 2007, 48(7), 2997-3004)). The compounds of the invention have been shown to give better results relating to one or more of the above properties compared to the compounds disclosed in WO 2010/074588.

The term ‘C_{x-y} alkyl’ as used herein refers to a linear or branched saturated hydrocarbon group containing from x to y carbon atoms. For example, C₁₋₄ alkyl refers to a linear or branched saturated hydrocarbon group containing from 1 to 4 carbon atoms. Examples of C₁₋₄ alkyl groups include methyl, ethyl, n-propyl, isopropyl, n-butyl, isobutyl, sec-butyl and tert-butyl. Preferably, the hydrocarbon group is linear.

The term ‘C_{x-y} alkoxy’ as used herein refers to an —O—C_{x-y} alkyl group wherein C_{x-y} alkyl is as defined above. Examples of such groups include methoxy, ethoxy, propoxy and butoxy. Where adjacent atoms in a chain are both substituted with an alkoxy group, the alkyl part of each alkoxy group can be joined so as to form a ring structure such as a dioxolyl group. For example, adjacent carbon atoms in a phenyl group can both be substituted with an alkoxy group with the alkyl parts joined so as to form benzo[d][1,3]dioxol-5-yl.

The term ‘aryl’ as used herein refers to a C₆₋₁₂ monocyclic or bicyclic hydrocarbon ring wherein at least one ring is aromatic. Examples of such groups include phenyl, naphthalenyl and tetrahydronaphthalenyl.

The term ‘halogen’ as used herein refers to a fluorine, chlorine, bromine or iodine atom, unless otherwise specified. In preferred embodiments, the halogen may be selected from fluorine, chlorine and bromine.

‘Pharmaceutically acceptable salts’ of the compounds of the present invention include salts with inorganic bases, salts with organic bases, salts with inorganic acids, salts with organic acids and salts with basic or acidic amino acids. Salts with acids may, in particular, be employed in some instances. Exemplary salts include hydrochloride, hydrobromide, hydroiodide, sulphate (mono- and di-), phosphate (mono-, di- and tri-), nitrate, carbonate, formate, acetate, propionate, pamoate, maleate, fumarate, succinate, tartrate [(D), (L), meso], citrate, mesylate, tosylate, aspartate and saccharate. The compounds of the present invention may be in either solvate (e.g. hydrate) or non-solvate (e.g. non-hydrate) form. When in a solvate form, additional solvents may be alcohols such as propan-2-ol.

General methods for the preparation of salts are well known to the person skilled in the art. Pharmaceutical acceptability of salts will depend on a variety of factors, including formulation processing characteristics and in vivo behaviour, and the skilled person would readily be able to assess such factors having regard to the present disclosure.

Where compounds of the invention exist in different enantiomeric and/or diastereoisomeric forms (including geometric isomerism about a double bond), these compounds may be prepared as isomeric mixtures or racemates, although the invention relates to all such enantiomers or isomers, whether present in an optically pure form or as mixtures with other isomers. Individual enantiomers or isomers may be obtained by methods known in the art, such as optical resolution of products or intermediates (for example chiral chromatographic separation (e.g. chiral HPLC)), or an enantioselective synthetic approach. Similarly, where compounds of the invention may exist as alternative tautomeric forms (e.g. keto/enol, amide/imidic acid), the invention relates to the individual tautomers in isolation, and to mixtures of the tautomers in all proportions. It has been found that the presence of a hydroxyl group as a substituent on the ring comprising R1 and R2 in Formula I helps to provide the compounds with good peripheral selectivity. Further, it has been found that CYP enzymes, which are responsible for drug metabolism in the human body, can metabolise at least some of the compounds of the invention to provide the necessary hydroxyl group. For example, if R1 and R2 are both hydrogen, it has been found that CYP enzymes will oxidise the compound so that a hydroxyl group is added to the phenyl ring on the right hand side of the compound. For example, see below:

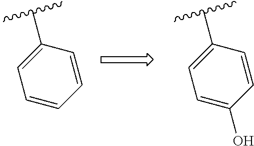 [SMILES: *c1ccccc1 ; *c1ccc(O)cc1]

Further, if R1 and/or R2 are C₁₋₄ alkoxy, the alkoxy group is metabolised to a hydroxyl group. For example, see below:

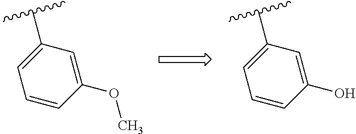

In addition, a compound may be peripherally selective as a result of having a relatively short half-life. For example, a compound which, in theory, could cross the blood brain barrier to enter central nervous system tissue may be metabolised relatively quickly so that the compound has been converted to an inactive form before it can cross the blood brain barrier and cause FAAH inhibition in the central nervous system.

The aryl group of R4 on the left hand side of the compound of Formula I is substituted with OSO₂NH₂, NHCONH₂, NHSO₂NH₂, NHSO₂C₁₋₄ alkyl or CONH₂. It has been found that these substituents, in combination with the aryl group, provide a relatively large polar surface area which helps to ensure that the compounds are confined to peripheral tissue. Furthermore, the protonable N-benzyl or N-phenyl motif on the right hand side of the compounds of Formula I helps to make the compounds water soluble. These properties help to make the compounds of the invention especially suitable for certain conditions, in particular, ocular conditions.

In addition, the presence of a hydroxyl group or a methoxy group on the right hand side of the compound, i.e. at R1 or R2, has also been found to provide improved water solubility of the compounds. Therefore, such compounds show advantageous properties.

As mentioned above, the compounds are relatively water soluble. In particular embodiments, the compounds have a water solubility of more than 4 mg/ml (in purified water having a pH of 5.6-5.8 at room temperature (see the examples for more details). In various embodiments, the compounds have a water solubility of more than 4 mg/ml. In some embodiments, the compounds have a water solubility of more than 6 mg/ml. In certain embodiments, the compounds have a water solubility of more than 8 mg/ml. In particular embodiments, the compounds have a water solubility of more than 10 mg/ml. In various embodiments, the compounds have a water solubility of more than 11 mg/ml. In some embodiments, the compounds have a water solubility of more than 12 mg/ml. In certain embodiments, the compounds have a water solubility of more than 13 mg/ml. In particular embodiments, the compounds have a water solubility of more than 14 mg/ml. In various embodiments, the compounds have a water solubility of more than 15 mg/ml. In some embodiments, the compounds have a water solubility of more than 16 mg/ml.

R1 is selected from hydrogen, halogen (such as fluorine), hydroxyl and C₁₋₄ alkoxy. In particular embodiments, R1 is preferably selected from hydrogen, hydroxyl and C₁₋₄ alkoxy. In various embodiments, R1 is selected from hydrogen, hydroxyl and C₁₋₃ alkoxy. In some embodiments, R1 is selected from hydrogen, hydroxyl and C₁₋₂ alkoxy. In certain embodiments, R1 is selected from hydrogen, hydroxyl and methoxy. In particular embodiments, R1 is selected from hydroxyl and C₁₋₄ alkoxy. In various embodiments, R1 is selected from hydroxyl and C₁-3 alkoxy. In some embodiments, R1 is selected from hydroxyl and C₁₋₂ alkoxy. In certain embodiments, R1 is selected from hydroxyl and methoxy. In particular embodiments, R1 is hydroxyl. In other embodiments, R1 is C₁₋₄ alkoxy. Further, R1 may be C₁₋₃ alkoxy. Additionally, R1 may be C₁₋₂ alkoxy. In certain embodiments, R1 is methoxy.

In various embodiments, R2 is selected from hydrogen, halogen, hydroxyl and C₁-3 alkoxy. In some embodiments, R2 is selected from hydrogen, halogen, hydroxyl and C₁₋₂ alkoxy. In particular embodiments, R2 is selected from hydrogen, halogen, hydroxyl and methoxy. R2 may be selected from hydrogen, fluorine, chlorine, hydroxyl and C₁₋₄ alkoxy. In various embodiments, R2 is selected from hydrogen, fluorine, chlorine, hydroxyl and C₁₋₃ alkoxy. In some embodiments, R2 is selected from hydrogen, fluorine, chlorine, hydroxyl and C₁₋₂ alkoxy. In particular embodiments, R2 is selected from hydrogen, fluorine, chlorine, hydroxyl and methoxy. R2 may be selected from hydrogen, fluorine, hydroxyl and C₁₋₄ alkoxy. In various embodiments, R2 is selected from hydrogen, fluorine, hydroxyl and C₁₋₃ alkoxy. In certain embodiments, R2 is selected from hydrogen, fluorine, hydroxyl and C₁₋₂ alkoxy. In particular embodiments, R2 is selected from hydrogen, fluorine, hydroxyl and methoxy. In various embodiments, R2 is hydrogen. In other embodiments, R2 is hydroxyl. In further embodiments, R2 is halogen. In certain embodiments, R2 is fluorine or chlorine. In some embodiments, R2 is fluorine. In particular embodiments, R2 is C₁₋₄ alkoxy. Further, R2 may be C₁₋₃alkoxy. Additionally, R2 may be C₁₋₂ alkoxy. In certain embodiments, R2 is methoxy.

In particular embodiments, R1 is selected from hydroxyl and C₁₋₄ alkoxy and R2 is selected from hydrogen, halogen, hydroxyl and C₁₋₄ alkoxy. In specific embodiments, R1 is selected from hydroxyl and C₁₋₄ alkoxy and R2 is selected from halogen, hydroxyl and C₁₋₄alkoxy. In particular embodiments, R1 is selected from hydroxyl and C₁₋₄ alkoxy and R2 is selected from hydrogen, hydroxyl and C₁₋₄ alkoxy. In some embodiments, R1 is selected from hydroxyl and C₁₋₄ alkoxy and R2 is selected from hydrogen, halogen and C₁₋₄ alkoxy. In various embodiments, R1 is selected from hydroxyl and C₁₋₄ alkoxy and R2 is selected from hydrogen, halogen and hydroxyl.

In particular embodiments, R1 is hydroxyl and R2 is selected from hydrogen, halogen (such as fluorine) and hydroxyl. In other embodiments, R1 is hydroxyl and R2 is selected from halogen (such as fluorine) and hydroxyl. In certain embodiments, R1 is hydroxyl and R2 is selected from hydrogen and hydroxyl. In various embodiments, R1 is hydroxyl and R2 is selected from hydrogen and halogen (such as fluorine).

In some embodiments, R1 and R2 are both hydroxyl. In some embodiments, R1 is hydroxyl and R2 is hydrogen. In further embodiments, R1 is hydroxyl and R2 is halogen (such as fluorine).

In particular embodiments, R1 is C₁₋₄ alkoxy (such as methoxy) and R2 is selected from hydrogen, halogen (such as fluorine) and C₁₋₄ alkoxy (such as methoxy). In other embodiments, R1 is C₁₋₄ alkoxy (such as methoxy) and R2 is selected from halogen (such as fluorine) and C₁₋₄ alkoxy (such as methoxy). In certain embodiments, R1 is C₁₋₄ alkoxy (such as methoxy) and R2 is selected from hydrogen and C₁₋₄ alkoxy (such as methoxy). In various embodiments, R1 is C₁₋₄ alkoxy (such as methoxy) and R2 is selected from hydrogen and halogen (such as fluorine).

In some embodiments, R1 and R2 are both C₁₋₄ alkoxy (such as methoxy). In some embodiments, R1 is C₁₋₄ alkoxy (such as methoxy) and R2 is hydrogen. In further embodiments, R1 is C₁₋₄ alkoxy (such as methoxy) and R2 is halogen such as fluorine.

In the embodiments above, when R1 is hydroxyl (but R1 is not limited to hydroxyl), R2 may be selected from hydrogen, halogen and hydroxyl. In some embodiments, when R1 is hydroxyl (but R1 is not limited to hydroxyl), R2 may be selected from hydrogen and halogen.

In the embodiments above, when R1 is C₁₋₄ alkoxy, such as methoxy, (but R1 is not limited to C₁₋₄ alkoxy), R2 may be selected from hydrogen, halogen and C₁₋₄ alkoxy, such as methoxy. Where two C₁₋₄ alkoxy groups are present, these may form a ring structure.

In particular embodiments, R3 is C₁₋₃ alkyl. In some embodiments, R3 is C₁₋₂ alkyl. In preferred embodiments, R3 is methyl.

R4 is aryl which is substituted with a group selected from OSO₂NH₂, NHCONH₂, NHSO₂NH₂, NHSO₂C₁₋₄ alkyl and CONH₂. In certain embodiments, R4 is aryl which is substituted with a group selected from OSO₂NH₂, NHCONH₂, NHSO₂NH₂, NHSO₂C₁₋₃ alkyl and CONH₂. In some embodiments, R4 is aryl which is substituted with a group selected from OSO₂NH₂, NHCONH₂, NHSO₂NH₂, NHSO₂C₁₋₂alkyl and CONH₂. In particular embodiments, R4 is aryl which is substituted with a group selected from OSO₂NH₂, NHCONH₂, NHSO₂NH₂, NHSO₂CH₃ and CONH₂.

In various embodiments, R4 is phenyl which is substituted with a group selected from OSO₂NH₂, NHCONH₂, NHSO₂NH₂, NHSO₂C₁₋₄ alkyl and CONH₂. In certain embodiments, R4 is phenyl which is substituted with a group selected from OSO₂NH₂, NHCONH₂, NHSO₂NH₂, NHSO₂C₁₋₃ alkyl and CONH₂. In some embodiments, R4 is phenyl which is substituted with a group selected from OSO₂NH₂, NHCONH₂, NHSO₂NH₂, NHSO₂C₁₋₂ alkyl and CONH₂. In particular embodiments, R4 is phenyl which is substituted with a group selected from OSO₂NH₂, NHCONH₂, NHSO₂NH₂, NHSO₂CH₃ and CONH₂.

R4 is substituted with a group selected from OSO₂NH₂, NHCONH₂, NHSO₂NH₂, NHSO₂C₁₋₄ alkyl and CONH₂. R4 may be substituted with a group selected from OSO₂NH₂, NHCONH₂, NHSO₂NH₂ and CONH₂. R4 may be substituted with a group selected from OSO₂NH₂, NHCONH₂, NHSO₂C₁₋₄ alkyl and CONH₂. R4 may be substituted with a group selected from OSO₂NH₂, NHCONH₂, NHSO₂NH₂ and NHSO₂C₁₋₄ alkyl. R4 may be substituted with a group selected from OSO₂NH₂, NHCONH₂ and CONH₂. R4 may be substituted with a group selected from OSO₂NH₂ and NHCONH₂. The options described in this paragraph are also applicable to R5 described below.

In various embodiments, R4 is aryl which is substituted with OSO₂NH₂. In some embodiments, R4 is aryl which is substituted with NHCONH₂. In other embodiments, R4 is aryl which is substituted with NHSO₂NH₂. In certain embodiments, R4 is aryl which is substituted with NHSO₂C₁₋₄ alkyl. In a number of embodiments, R4 is aryl which is substituted with NHSO₂C₁₋₃ alkyl. In certain embodiments, R4 is aryl which is substituted with NHSO₂C₁₋₂ alkyl. In particular embodiments, R4 is aryl which is substituted with NHSO₂CH₃. In various embodiments, R4 is aryl which is substituted with CONH₂.

In various embodiments, R4 is phenyl which is substituted with OSO₂NH₂. In some embodiments, R4 is phenyl which is substituted with NHCONH₂. In other embodiments, R4 is phenyl which is substituted with NHSO₂NH₂. In certain embodiments, R4 is phenyl which is substituted with NHSO₂C₁₋₄ alkyl. In a number of embodiments, R4 is phenyl which is substituted with NHSO₂C₁₋₃ alkyl. In certain embodiments, R4 is phenyl which is substituted with NHSO₂C₁₋₂ alkyl. In particular embodiments, R4 is phenyl which is substituted with NHSO₂CH₃. In various embodiments, R4 is phenyl which is substituted with CONH₂.

In embodiments in which R4 is aryl substituted with OSO₂NH₂, the OSO₂NH₂ group is preferably at the meta or para position. In particular, when R4 is phenyl substituted with OSO₂NH₂, the OSO₂NH₂ group is preferably at the meta or para position.

In embodiments in which R4 is aryl substituted with NHCONH₂, the NHCONH₂ group is preferably at the meta position. In particular, when R4 is phenyl substituted with NHCONH₂, the NHCONH₂ group is preferably at the meta position.

In embodiments in which R4 is aryl substituted with NHSO₂NH₂, the NHSO₂NH₂ group is preferably at the meta position. In particular, when R4 is phenyl substituted with NHSO₂NH₂, the NHSO₂NH₂ group is preferably at the meta position. It has been found that having the NHSO₂NH₂ group in the meta position rather than the para position reduces the genotoxicity risk of the compound.

In embodiments in which R4 is aryl substituted with NHSO₂C₁₋₄ alkyl (or NHSO₂C₁₋₃ alkyl, NHSO₂C₁₋₂ alkyl or NHSO₂CH₃), the NHSO₂C₁₋₄ alkyl group (or NHSO₂C₁₋₃ alkyl, NHSO₂C₁₋₂ alkyl or NHSO₂CH₃) is preferably at the meta position. In particular, when R4 is phenyl substituted with NHSO₂C₁₋₄ alkyl (or NHSO₂C₁₋₃ alkyl, NHSO₂C₁₋₂ alkyl or NHSO₂CH₃), the NHSO₂C₁₋₄ alkyl group (or NHSO₂C₁₋₃ alkyl, NHSO₂C₁₋₂ alkyl or NHSO₂CH₃) is preferably at the meta position.

In embodiments in which R4 is aryl substituted with CONH₂, the CONH₂ group is preferably at the meta or para position. In particular, when R4 is phenyl substituted with CONH₂, the CONH₂ group is preferably at the meta or para position. More preferably, the CONH₂ group is at the meta position. In particular, when R4 is phenyl substituted with CONH₂, the CONH₂ group is preferably at the meta position.

In the compound of the invention, m is 0 or 1. This means that when m is 0, the ring structure is pyrrolidinyl and when m is 1, the ring structure is piperidinyl.

In a particularly preferred embodiment, m is 1 in the compound of the invention. Therefore, the present invention provides a compound having Formula Ia:

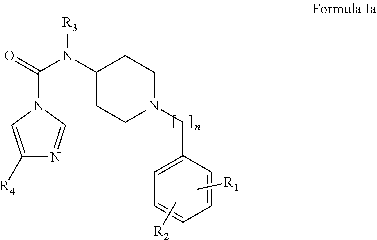

wherein:
R1 is selected from hydrogen, halogen, hydroxyl and C₁₋₄ alkoxy;
R2 is selected from hydrogen, halogen, hydroxyl and C₁₋₄ alkoxy;
R3 is C₁₋₄ alkyl;
R4 is aryl which is substituted with a group selected from OSO₂NH₂, NHCONH₂, NHSO₂NH₂, NHSO₂C₁₋₄ alkyl and CONH₂; and
n is 0 or 1;
or a pharmaceutically acceptable salt thereof;
provided that the compound is not N-(1-benzylpiperidin-4-yl)-N-methyl-4-(4-(sulfamoylamino)phenyl)-1H-imidazole-1-carboxamide or N-(1-benzylpiperidin-4-yl)-N-methyl-4-(3-(methylsulfonamido)phenyl)-1H-imidazole-1-carboxamide.

The preference of m being 1 also applies to all embodiments and Formulae described below.

In the compound of the invention, n is 0 or 1. This means that when n is 0, a bond is present between the ring nitrogen atom and the phenyl ring and when n is 1, a CH₂ moiety is present between the ring nitrogen atom and the phenyl ring to form a benzyl moiety.

In particular embodiments, when m is 0, n is 0. In some embodiments, when m is 0, n is not 1. Further, when m is 1, n may be 0 or 1. In specific embodiments, when m is 1, n is 0. In other embodiments, when m is 1, n is 1.

It is specifically envisaged that any option described above can be combined with any other option described above. Therefore, the various options for R1, R2, R3, R4, m and n can be combined in any way and all such combinations are specifically envisaged. Further, for the avoidance of doubt, it is specifically envisaged that the various options for R1 can be combined and that the various options for R2 can be combined. Furthermore, it is also specifically envisaged that the various options or combinations for R1 can be combined with the various options and combinations for R2.

In a preferred embodiment, there is provided a compound having Formula II:

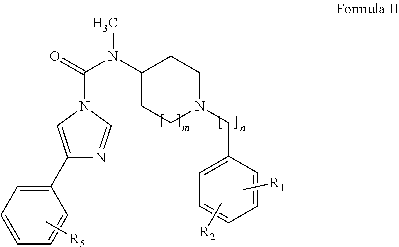

wherein:
R1 is selected from hydrogen, hydroxyl and C₁₋₄ alkoxy;
R2 is selected from hydrogen, halogen, hydroxyl and C₁₋₄ alkoxy;
R5 is selected from OSO₂NH₂, NHCONH₂, NHSO₂NH₂, NHSO₂C₁₋₄ alkyl and CONH₂;
m is 0 or 1; and
n is 0 or 1;
or a pharmaceutically acceptable salt thereof;
provided that the compound is not N-(1-benzylpiperidin-4-yl)-N-methyl-4-(4-(sulfamoylamino)phenyl)-1H-imidazole-1-carboxamide or N-(1-benzylpiperidin-4-yl)-N-methyl-4-(3-(methylsulfonamido)phenyl)-1H-imidazole-1-carboxamide.

The options described above for Formula I for R1, R2, m and n also apply to above Formula II. Further, the options for R4 described above for Formula I, where R4 is a substituted phenyl, are equally applicable to R5 in Formula II.

In various embodiments, R5 is OSO₂NH₂. In some embodiments, R5 is NHCONH₂. In other embodiments, R5 is NHSO₂NH₂. In certain embodiments, R5 is NHSO₂C₁₋₄ alkyl. In a number of embodiments, R5 is NHSO₂C₁₋₃ alkyl. In certain embodiments, R5 is NHSO₂C₁₋₂ alkyl. In particular embodiments, R5 is NHSO₂CH₃. In various embodiments, R5 is CONH₂. Each of these R5 groups may be in a particular position on the phenyl ring and the preferred position is described above with respect to R4.

In another preferred embodiment, there is provided a compound having Formula III:

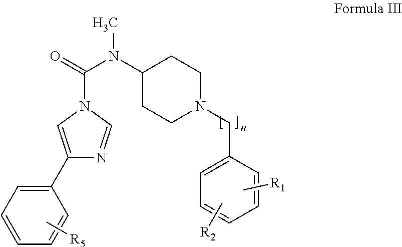

wherein:
R1 is selected from hydrogen, hydroxyl and C₁₋₄ alkoxy;
R2 is selected from hydrogen, halogen, hydroxyl and C₁₋₄ alkoxy;
R5 is selected from OSO₂NH₂, NHCONH₂, NHSO₂NH₂, NHSO₂C₁₋₄ alkyl and CONH₂;
and
n is 0 or 1;
or a pharmaceutically acceptable salt thereof;
provided that the compound is not N-(1-benzylpiperidin-4-yl)-N-methyl-4-(4-(sulfamoylamino)phenyl)-1H-imidazole-1-carboxamide or N-(1-benzylpiperidin-4-yl)-N-methyl-4-(3-(methylsulfonamido)phenyl)-1H-imidazole-1-carboxamide.

The options described above for Formula I for R1, R2 and n also apply to above Formula III. Further, the options for R4 described above for Formula I, where R4 is a substituted phenyl, are equally applicable to R5 in Formula III.

In a further preferred embodiment, there is provided a compound having Formula IV:

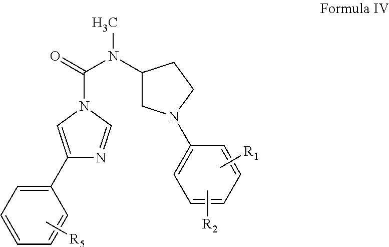

wherein:
R1 is selected from hydrogen, hydroxyl and C₁₋₄ alkoxy;
R2 is selected from hydrogen, halogen, hydroxyl and C₁₋₄ alkoxy; and
R5 is selected from OSO₂NH₂, NHCONH₂, NHSO₂NH₂, NHSO₂C₁₋₄ alkyl and CONH₂;
or a pharmaceutically acceptable salt thereof.

The options described above for Formula I for R1 and R2 also apply to above Formula IV. Further, the options for R4 described above for Formula I, where R4 is a substituted phenyl, are equally applicable to R5 in Formula IV.

In a particularly preferred embodiment, the present invention provides a compound having Formula V:

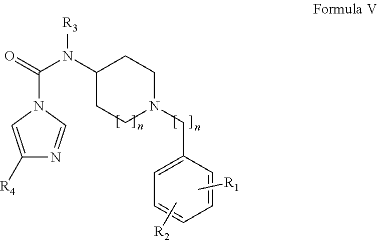

wherein:
R1 is selected from hydroxyl and C₁₋₄ alkoxy;
R2 is selected from hydrogen, halogen, hydroxyl and C₁₋₄ alkoxy;
R3 is C₁₋₄ alkyl;
R4 is aryl which is substituted with a group selected from OSO₂NH₂, NHCONH₂, NHSO₂NH₂, NHSO₂C₁₋₄ alkyl and CONH₂;
m is 0 or 1; and
n is 0 or 1;
or a pharmaceutically acceptable salt thereof.

The options described above for Formula I for R1, R2, R3, R4, m and n also apply to above Formula V with the exception that R1 cannot be hydrogen, unlike in Formula I.

In another particularly preferred embodiment, the present invention provides a compound having Formula VI:

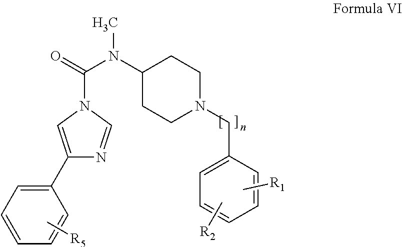

wherein:
R1 is selected from hydrogen, halogen, hydroxyl and C₁₋₄ alkoxy;
R2 is selected from hydrogen, halogen, hydroxyl and C₁₋₄ alkoxy;
R5 is selected from OSO₂NH₂, NHCONH₂, NHSO₂NH₂, NHSO₂CH₃ and CONH₂; and
n is 0 or 1;
or a pharmaceutically acceptable salt thereof;
provided that the compound is not N-(1-benzylpiperidin-4-yl)-N-methyl-4-(4-(sulfamoylamino)phenyl)-1H-imidazole-1-carboxamide or N-(1-benzylpiperidin-4-yl)-N-methyl-4-(3-(methylsulfonamido)phenyl)-1H-imidazole-1-carboxamide.

The options described above for the various embodiments for R1, R2, R5 and n also apply to Formula VI.

In some embodiments for Formula VI, R1 is selected from hydrogen, hydroxyl and C₁₋₄ alkoxy (e.g. methoxy); and R2 is selected from hydrogen, halogen, hydroxyl and C₁₋₄ alkoxy (e.g. methoxy).

In certain embodiments for Formula VI, R1 is selected from hydroxyl and C₁₋₄ alkoxy (e.g. methoxy); and R2 is selected from hydrogen, halogen, hydroxyl and C₁₋₄ alkoxy (e.g. methoxy).

In particular embodiments for Formula VI, R1 is selected from hydroxyl and C₁₋₄ alkoxy (e.g. methoxy); and R2 is selected from hydrogen, halogen and C₁₋₄ alkoxy (e.g. methoxy).

In accordance with a second aspect of the invention, there is provided a pharmaceutical composition comprising a compound according to the first aspect of the invention, together with one or more pharmaceutically acceptable excipients.

Pharmaceutical compositions of this invention comprise the compound of the first aspect of the present invention with any pharmaceutically acceptable carrier, adjuvant or vehicle. The pharmaceutical compositions may contain any conventional non-toxic pharmaceutically-acceptable carriers, adjuvants or vehicles. Pharmaceutically acceptable carriers, adjuvants and vehicles that may be used in the pharmaceutical compositions of this invention are those conventionally employed in the field of pharmaceutical formulation, and include, but are not limited to, sugars, sugar alcohols, starches, ion exchangers, alumina, aluminium stearate, lecithin, serum proteins, such as human serum albumin, buffer substances such as phosphates, glycerine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, intravenously or topically.

The pharmaceutical compositions of this invention may be orally administered in any orally acceptable dosage form including, but not limited to, capsules, tablets, powders, granules, and aqueous suspensions and solutions. These dosage forms are prepared according to techniques well-known in the art of pharmaceutical formulation. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavouring and/or colouring agents may be added.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as that described in Ph. Helv, or a similar alcohol.

The compounds of the present invention may be administered in a dose of around 1 to around 20,000 μg/kg per dose, for example, around 1 to around 10,000 μg/kg, around 1 to around 5,000 μg/kg, around 1 to around 3,000 μg/kg, around 1 to around 2,000 μg/kg, around 1 to around 1,500 μg/kg, around 1 to around 1,000 μg/kg, around 1 to around 500 μg/kg, around 1 to around 250 μg/kg, around 1 to around 100 μg/kg, around 1 to around 50 μg/kg or around 1 to around 25 μg/kg per dose depending on the condition to be treated or prevented, and the characteristics of the subject being administered with the compound. In many instances, the dose may be around 1 to around 10 μg/kg per dose. In particular embodiments, the dose may be around 250 μg/kg per dose, around 100 μg/kg, around 50 μg/kg or around 10 μg/kg per dose. The dosing regimen for a given compound could readily be determined by the skilled person having access to this disclosure.

In one particular embodiment, the pharmaceutical composition of the invention additionally comprises one or more additional active pharmaceutical ingredients. The compound of the invention may be administered with one or more additional active pharmaceutical ingredients, such as anandamide, N-oleoylethanolamine or N-palmitoylethanolamine. This may be in the form of a single composition comprising the compound of the invention and one or more additional active pharmaceutical ingredients. Alternatively, this may be in two or more separate compositions where the compound of the invention is contained in one composition and the one or more additional active pharmaceutical ingredients are contained in one or more separate compositions.

Administration of the compounds of the present invention may therefore be simultaneous with, or staggered with respect to, the one or more additional active pharmaceutical ingredient.

In a third aspect, the present invention provides a compound according to the first aspect of the invention, or a composition according to the second aspect, for use in therapy.

In a fourth aspect, the invention provides a compound according to the first aspect of the invention, or a composition according to the second aspect, for use in the treatment or prevention of a condition, for example an ocular condition, whose development or symptoms are linked to a substrate of the FAAH enzyme.

The invention also provides the use of a compound according to the first aspect of the invention, or a composition according to the second aspect, in the manufacture of a medicament for the treatment or prevention of a condition, for example an ocular condition, whose development or symptoms are linked to a substrate of the FAAH enzyme.

A number of ocular conditions whose development or symptoms are linked to a substrate of the FAAH enzyme are known to the skilled person. These include ocular hypertension, retinopathy, glaucoma, ocular pain, chronic corneal pain, dry eye syndrome, post-surgical recovery and ocular inflammatory disorders such as uveitis, scleritis, episcleritis, episclera, keratitis, retinal vasculitis and chronic conjunctivitis.

Furthermore, other conditions whose development or symptoms are linked to a substrate of the FAAH enzyme include reduction of L-dopa-induced hyperactivity in adjunctive dopamine replacement therapy, bladder control and stress-related neuroinflammatory disorders such as post-traumatic stress disorder, multiple sclerosis and stroke. Systemic administration may be useful for such conditions. For example, see Johnston et al. The Journal of Pharmacology and Experimental Therapeutics 2011, 336(2), 423-430; Di Carlo et al. Expert Opin. Investig. Drugs 2003, 12(1), 39-49; Schicho et al. Expert Rev. Clin. Pharmacol. 2010, 3(2), 193-207; and Aizawa et al. The Journal of Urology 2014 Apr. 16, DOI: http://dx.doi.org/10.1016/j.juro.2014.04.008.

In a fifth aspect, the invention also provides a method of treatment or prevention of a condition, for example an ocular condition, whose development or symptoms are linked to a substrate of the FAAH enzyme, the method comprising the administration, to a subject in need of such treatment or prevention, of a therapeutically effective amount of a compound according to the first aspect of the invention, or a composition according to the second aspect.

A compound according to the fourth aspect, or a method according to the fifth aspect, wherein the condition, for example ocular condition, is a disorder associated with the endocannabinoid system.

### DETAILED DESCRIPTION OF THE INVENTION

The invention will now be described in more detail by way of example only:

### 1. Synthetic Methodologies

The methods used for synthesis of the compounds of the invention are illustrated by the general schemes below. The starting materials and reagents used in preparing these compounds are available from commercial suppliers or can be prepared by methods obvious to those skilled in the art. These general schemes are merely illustrative of methods by which the compounds of this invention can be synthesised, and various modifications to these schemes can be made and will be suggested to one skilled in the art having referred to this disclosure.

All compounds and intermediates were characterised by nuclear magnetic resonance (NMR). NMR spectra were recorded on a Bruker Avance III 600 MHz spectrometer with solvent used as internal standard. 13C spectra were recorded at 150 MHz and 1H spectra were recorded at 600 MHz. Data are reported in the following order: approximate chemical shift (ppm), number of protons, multiplicity (br, broad; d, doublet; m, multiplet; s, singlet, t; triplet) and coupling constant (Hz).

Room temperature in the following scheme means the temperature ranging from 20° C. to 25° C.

### Intermediate 1: 4-(3-nitrophenyl)-1H-imidazole

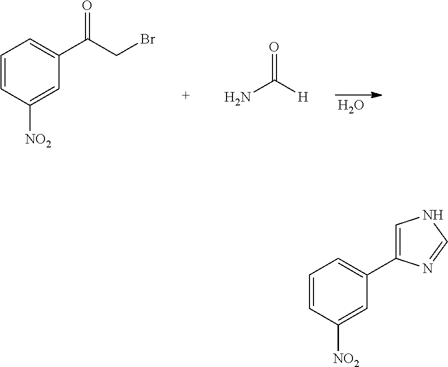

In a 50 mL pear flask 2-bromo-1-(3-nitrophenyl)ethanone (3 g, 12.29 mmol), formamide (6.08 ml, 152 mmol) and water (0.45 ml) were placed. The mixture was heated at 140° C. and stirred for 7 h. Then, it was cooled to room temperature and poured onto water. The precipitate was filtered off, washed with water. The filtrate's pH was set to 12 by adding 3N NaOH solution. The resultant precipitate was filtered off, washed with water and dried under vacuum. (Yield: 0.74 g, 30%).

### Intermediate 2: 4-(3-methoxyphenyl)-1H-imidazole

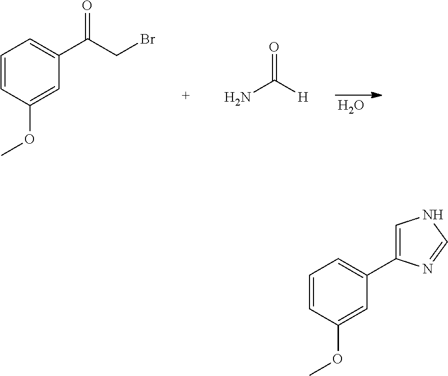

In a 50 mL pear flask 2-Bromo-3′-methoxyacetophenone (4.3 g, 18.77 mmol), formamide (9.28 mL, 233 mmol) and water (0.69 mL) were placed. The mixture was heated at 140° C. and stirred for 4 hours. Then, it was cooled to room temperature and poured into water (100 mL). The precipitate was filtered off and washed with water. The filtrate's pH was set to 12 by adding 3N NaOH solution. The resultant precipitate was filtered off, washed with water and dried under vacuum. (Yield: 1.45 g, 44%).

### Intermediate 3: 4-(4-methoxyphenyl)-1H-imidazole

The title compound, was prepared by analogous manner to intermediate 2 from 1-(4-methoxyphenyl)ethanone.

### Intermediate 4: N-methyl-1-phenylpiperidin-4-amine

### Step1: 1-phenylpiperidin-4-one

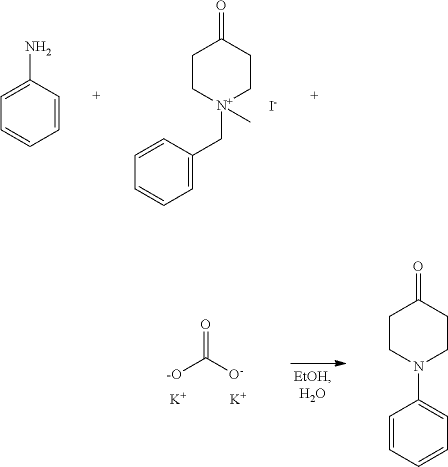

In a 250 mL round-bottomed flask aniline (2.84 mL, 31.1 mmol), potassium carbonate (0.603 g, 4.36 mmol) and ethanol (57 mL) were placed. The mixture was heated at reflux and a suspension of 1-benzyl-1-methyl-4-oxopiperidinium iodide (15.69 g, 47.4 mmol) in water (43 mL) was added over a period of 1 h. The reaction mixture was stirred at reflux for 45 min. Then, it was quenched with water (175 mL) and the solution was extracted with dichloromethane. The organic phase was dried over MgSO₄, filtered and evaporated. The obtained yellow oil was separated by column chromatography (petroleum ether/ethyl acetate, 9:1, 4:1). (Yield: 4.67 g, 81%).

### Step2: N-methyl-1-phenylpiperidin-4-amine

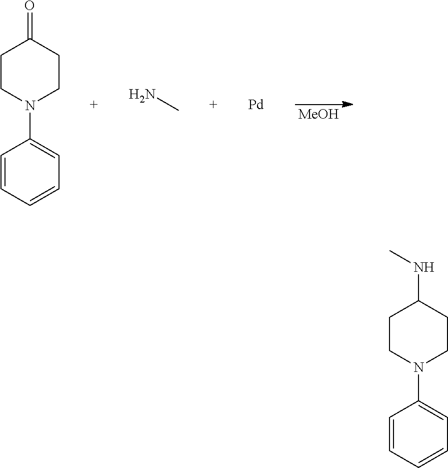

In a 50 mL pear flask, 1-phenylpiperidin-4-one (1.52 g, 8.67 mmol) and methanol (15.2 mL) were placed under inert atmosphere. Methylamine (3.78 mL, 38.2 mmol) was added, followed by palladium (10% on carbon, 0.138 g, 0.130 mmol). The reaction flask was placed in an autoclave and was charged with 20 atm hydrogen. The autoclave was heated at 50° C. and stirred for 2 h. The reaction mixture was filtered through celite and then the solvent was removed. The resultant pale yellow oil crystallized on standing. (Yield: 1.58 g, 91%).

### Intermediate 5: 1-(4-methoxyphenyl)-N-methylpiperidin-4-amine

The title compound was prepared by analogous manner to intermediate 4 from 4-methoxyaniline.

### Intermediate 6: N-methyl-1-phenylpiperidin-4-amine

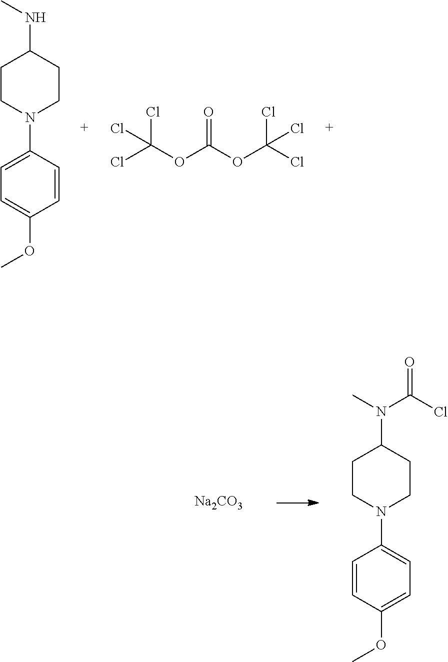 [SMILES: CNC1CCN(c2ccc(OC)cc2)CC1 ; O=C(OC(Cl)(Cl)Cl)OC(Cl)(Cl)Cl ; "Na2CO3" ; COc1ccc(N2CCC(N(C)C(=O)Cl)CC2)cc1]

A cooled solution (0° C.) of bis(trichloromethyl)carbonate (1.078 g, 3.63 mmol) in dichloromethane (10 mL) was treated with a solution of 1-(4-methoxyphenyl)-N-methylpiperidin-4-amine (intermediate 5) (2 g, 9.08 mmol) in dichloromethane (10 mL). Then, sodium carbonate (1.924 g, 18.16 mmol) was added portionwise. The reaction mixture was allowed to warm up to room temperature and stirred for 3 h. Then, it was quenched with water. The organic phase was separated and dried over MgSO₄. The solvent was removed under reduced pressure and the resulting solid was triturated with petroleum ether, filtered and dried under vacuum. (Yield: 2.11 g, 82%).

### Intermediate 7: 4-(3-aminophenyl)-N-(1-benzylpiperidin-4-yl)-N-methyl-1H-imidazole-1-carboxamide

### Step1: phenyl 4-(3-nitrophenyl)-1H-imidazole-1-carboxylate

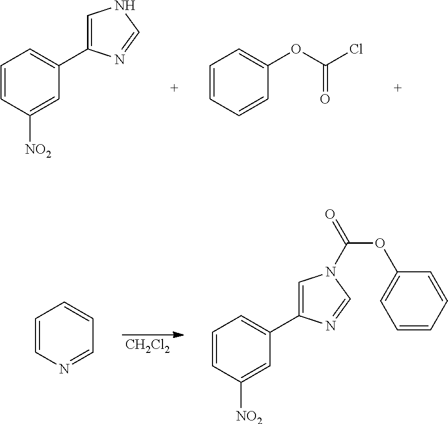

In a 500 mL round-bottomed flask 4-(3-nitrophenyl)-1H-imidazole (8.3 g, 43.9 mmol) and dichloromethane (400 ml) were placed under inert atmosphere. The suspension was cooled to 0° C. and was treated dropwise with pyridine (4.26 ml, 52.7 mmol), followed by addition of phenyl chloroformate (6.61 ml, 52.7 mmol). The mixture was allowed to warm up to room temperature and stirred for 4 h. The reaction mixture was quenched with ice-water. The phases were separated and the organic phase was washed with water, 1N HCl solution and saturated NaCl solution, respectively. The organic layer was dried over MgSO₄, filtered and evaporated. The resultant pale yellow solid was crystallized from a mixture of dichloromethane/isopropanol, filtered and dried under vacuum. (Yield: 11.79 g, 74%).

### Step2: N-(1-benzylpiperidin-4-yl)-N-methyl-4-(3-nitrophenyl)-1H-imidazole-1-carboxamide

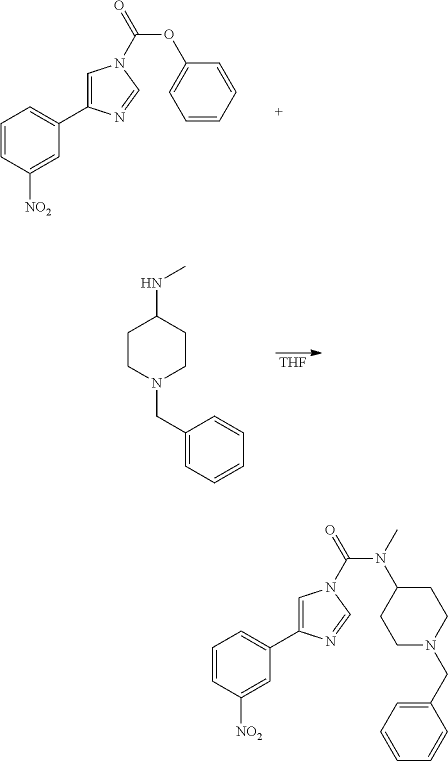 [SMILES: O=C(Oc1ccccc1)n1cnc(-c2cccc([N+](=O)[O-])c2)c1 ; CNC1CCN(Cc2ccccc2)CC1 ; CN(C(=O)n1cnc(-c2cccc([N+](=O)[O-])c2)c1)C1CCN(Cc2ccccc2)CC1 ; "THF"]

A solution of 1-benzyl-N-methylpiperidin-4-amine (1.40 g, 6.85 mmol) in tetrahydrofuran (2 mL) was added to a stirred solution of phenyl 4-(3-nitrophenyl)-1H-imidazole-1-carboxylate (1.06 g, 3.43 mmol) in tetrahydrofuran (18 mL) at room temperature. The reaction mixture was stirred at room temperature for 72 h. The solvent was evaporated to give a yellow solid that was recrystallized from a mixture of dichloromethane/ethanol. (Yield: 0.493 g, 34%).

### Step3: 4-(3-aminophenyl)-N-(1-benzylpiperidin-4-yl)-N-methyl-1H-imidazole-1-carboxamide

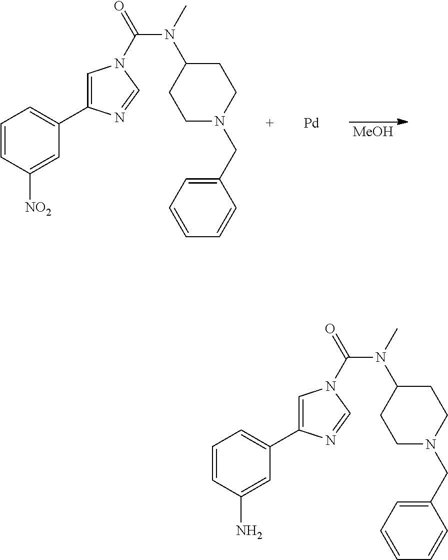

Palladium (10% on carbon, 0.061 g, 0.057 mmol) was added to a stirred suspension of N-(1-benzylpiperidin-4-yl)-N-methyl-4-(3-nitrophenyl)-1H-imidazole-1-carboxamide (0.481 g, 1.147 mmol) in methanol (50 ml) at room temperature, under inert atmosphere. The reaction mixture was allowed to stir at room temperature, under hydrogen atmosphere (at atmospheric pressure), for 40 min. Then, it was filtered through celite and the filter cake was washed with a mixture of ethyl acetate/methanol 1:1. The filtrate was evaporated to leave a pale yellow solid. The solid was recrystallised from isopropanol/dichloromethane. (Yield: 0.172 g, 38%).

### Intermediate 8: 4-(3-aminophenyl)-N-(1-(3-methoxybenzyl)piperidin-4-yl)-N-methyl-1H-imidazole-1-carboxamide

### Step1: tert-butyl 4-(N-methyl-4-(3-nitrophenyl)-1H-imidazole-1-carboxamido)piperidine-1-carboxylate

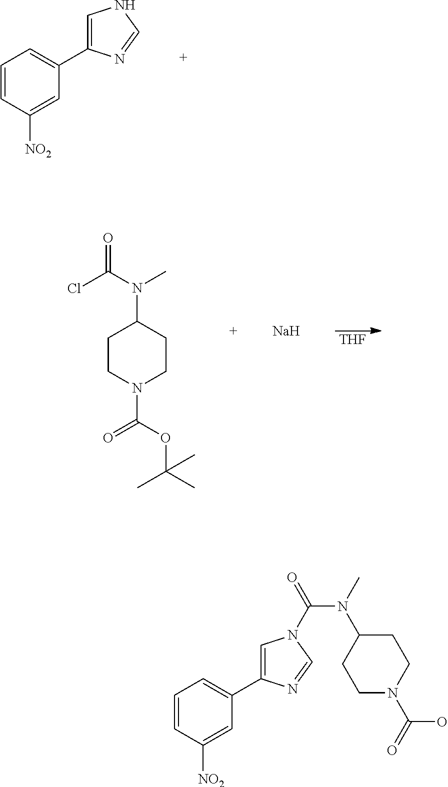

In a 250 mL round-bottomed flask 4-(3-nitrophenyl)-1H-imidazole (Intermediate 1) (5 g, 26.4 mmol) and anhydrous tetrahydrofuran (125 ml) were placed, under inert atmosphere. The mixture was cooled to 0° C. and sodium hydride (60% dispersion in mineral oil, 1.269 g, 31.7 mmol) was added portionwise. Then, the reaction mixture was allowed to warm up to room temperature and stirred for 30 min. Then, tert-butyl 4-(chlorocarbonyl(methyl)amino)piperidine-1-carboxylate (Intermediate 16) (10.97 g, 39.6 mmol) was added portionwise and the reaction mixture was allowed to stir for 2 h. The mixture was cooled to 0° C. and quenched with water. The phases were separated, the organic phase was diluted with ethyl acetate, washed with water, dried over MgSO₄, filtered and evaporated. The crude oil was crystallized from isopropanol. (Yield: 9.56 g, 84%).

### Step2: N-methyl-4-(3-nitrophenyl)-N-(piperidin-4-yl)-1H-imidazole-1-carboxamide hydrochloride

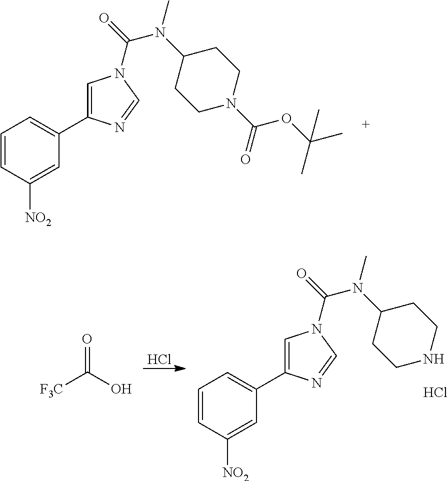 [SMILES: CN(C(=O)n1cnc(-c2cccc([N+](=O)[O-])c2)c1)C1CCN(C(=O)OC(C)(C)C)CC1 ; O=C(O)C(F)(F)F ; Cl ; CN(C(=O)n1cnc(-c2cccc([N+](=O)[O-])c2)c1)C1CCNCC1 ; Cl]

tert-butyl 4-(N-methyl-4-(3-nitrophenyl)-1H-imidazole-1-carboxamido)piperidine-1-carboxylate (4.3 g, 10.01 mmol) was dissolved in trifluoroacetic acid (35 mL), at 0° C., and the reaction was allowed to stir vigorously at room temperature for 1 h. Then, trifluoroacetic acid was removed under reduced pressure and the obtained residue was dissolved in 20 mL of methanol. The solution was cooled to 0° C. and treated with 2N hydrogen chloride solution in diethyl ether (5.51 mL, 11.01 mmol). Diethyl ether was then added dropwise until a white precipitate was formed. The precipitate was filtered, washed with diethyl ether and dried under vacuum. (Yield: 3.637 g, 99%).

### Step3: N-(1-(3-methoxybenzyl)piperidin-4-yl)-N-methyl-4-(3-nitrophenyl)-1H-imidazole-1-carboxamide

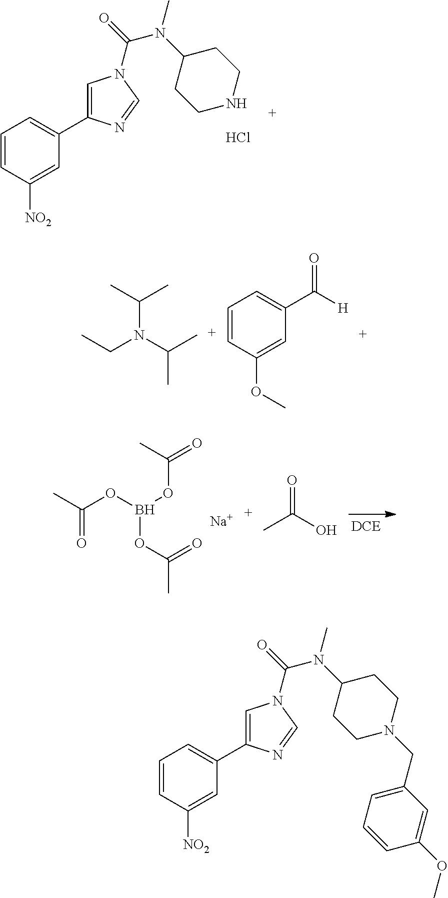

In a 100 mL round-bottomed flask N-methyl-4-(3-nitrophenyl)-N-(piperidin-4-yl)-1H-imidazole-1-carboxamide hydrochloride (2 g, 5.47 mmol) and dichloroethane (60 mL) were placed. N,N-diisopropylethylamine (3.82 ml, 21.87 mmol) was added, followed by 3-methoxybenzaldehyde (1.332 ml, 10.93 mmol). The mixture was stirred at room temperature for 30 min. and then sodium triacetoxyborohydride (2.317 g, 10.93 mmol) was added followed by acetic acid (0.313 ml, 5.47 mmol). After 5 h of stirring at room temperature, another portion of sodium triacetoxyborohydride (1.159 g, 5.47 mmol) was added and the mixture was further stirred overnight. The reaction mixture was quenched with water, and then NaHCO₃ saturated solution was added. The phases were separated and the aqueous phase was extracted with dichloromethane. The combined organic phases were washed with brine, dried over MgSO₄, filtered and evaporated. The pale yellow oil was triturated with diethyl ether. (Yield: 1.74 g, 71%).

### Step4: 4-(3-aminophenyl)-N-(1-(3-methoxybenzyl)piperidin-4-yl)-N-methyl-1H-imidazole-1-carboxamide

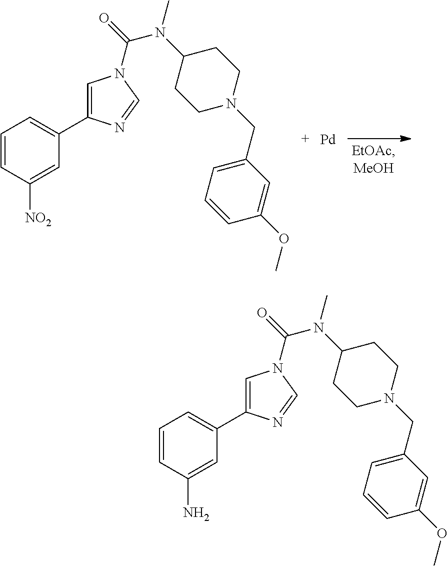

Palladium (10% on carbon, 0.314 g, 0.295 mmol) was added to a solution of N-(1-(3-methoxybenzyl)piperidin-4-yl)-N-methyl-4-(3-nitrophenyl)-1H-imidazole-1-carboxamide (2.65 g, 5.90 mmol) in a mixture of ethyl acetate (108 mL) and methanol (108 mL) at room temperature, under inert atmosphere. The reaction mixture was allowed to stir at room temperature, under hydrogen atmosphere (1 atm.), for 2 h. Thereupon, the reaction was filtered through celite, the filter cake was washed with a mixture of ethyl acetate/methanol 1:1. The filtrate was evaporated to give a pale yellow solid. The solid was recrystallised from isopropanol/dichloromethane. (Yield: 1.3 g, 50%).

### Intermediate 9: 4-(3-aminophenyl)-N-(1-(3,4-dimethoxybenzyl)piperidin-4-yl)-N-methyl-1H-imidazole-1-carboxamide

The title compound was prepared by analogous manner to intermediate 8 from 3,4-dimethoxy benzaldehyde.

### Intermediate 10: 4-(3-hydroxyphenyl)-N-methyl-N-(1-phenylpiperidin-4-yl)-1H-imidazole-1-carboxamide hydrobromide

### Step1: phenyl 4-(3-methoxyphenyl)-1H-imidazole-1-carboxylate

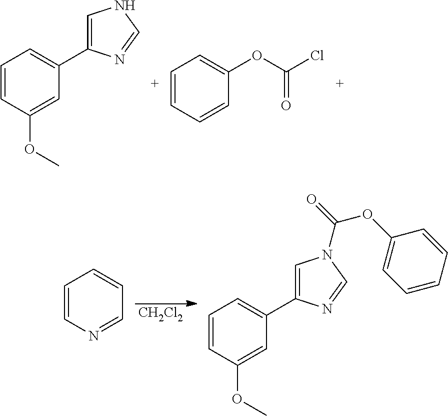

In a 250 mL round-bottomed flask 4-(3-methoxyphenyl)-1H-imidazole (Intermediate 2) (1.9 g, 10.91 mmol) and dichloromethane (95 mL) were placed, under inert atmosphere. The suspension was cooled to 0° C. and was treated dropwise with pyridine (1.059 mL, 13.09 mmol), followed by phenyl chloroformate (1.642 mL, 13.09 mmol). The mixture was allowed to warm up to room temperature and stirred for 1 h. The reaction mixture was quenched with ice, followed by water. The phases were separated and organic layer was washed with water, 1N HCl solution and saturated NaCl solution, respectively. The organic layer was dried over MgSO₄, filtered and evaporated. The resultant pale yellow solid was triturated with petroleum ether, filtered and dried under vacuum. (Yield: 2.9 g, 86%).

### Step2: phenyl 4-(3-methoxyphenyl)-1H-imidazole-1-carboxylate

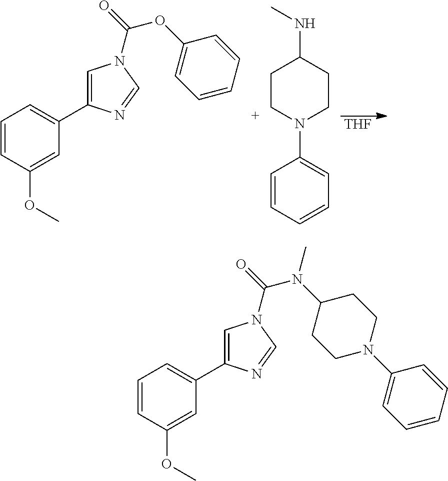

In a 100 mL round-bottomed flask, 4-(3-methoxyphenyl)-1H-imidazole-1-carboxylate (2 g, 6.80 mmol) and anhydrous tetrahydrofuran (75 mL) were placed under inert atmosphere. N-methyl-1-phenylpiperidin-4-amine (Intermediate 4) (2.59 g, 13.59 mmol) was added and the solution was stirred at room temperature for 24 h, then was heated at reflux for another 24 h. The reaction mixture was cooled to room temperature, filtered and the filtrate was evaporated. The solid was crystallized from tetrahydrofuran. (Yield: 1.23 g, 44%).

### Step3: 4-(3-hydroxyphenyl)-N-methyl-N-(1-phenylpiperidin-4-yl)-1H-imidazole-1-carboxamide hydrobromide

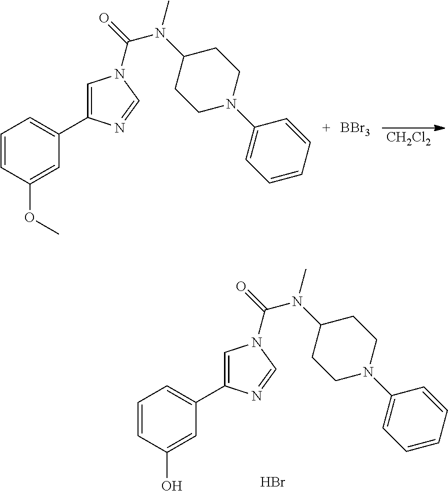

In a 100 mL round-bottomed flask, 4-(3-methoxyphenyl)-N-methyl-N-(1-phenylpiperidin-4-yl)-1H-imidazole-1-carboxamide (1.2 g, 3.07 mmol) and anhydrous dichloromethane (50 mL) were placed under inert atmosphere. The mixture was cooled to −78° C. and boron tribromide (0.872 mL, 9.22 mmol) was added dropwise. The reaction was stirred at −78° C. for 5 min, and then was allowed to warm up to room temperature and stirred for 4 h. The reaction mixture was cooled to 0° C., quenched with water and stirred for 15 min. The precipitate was filtered off, washed with water and dried under vacuum. The solid was crystallized from dichloromethane/isopropanol. (Yield: 0.984 g, 70%).

### Intermediate 11: N-(1-benzylpiperidin-4-yl)-4-(3-hydroxyphenyl)-N-methyl-1H-imidazole-1-carboxamide hydrobromide

The title compound was prepared by analogous manner to intermediate 10 from N-methyl-1-benzylpiperidin-4-amine.

### Intermediate 12: 4-(4-hydroxyphenyl)-N-methyl-N-(piperidin-4-yl)-1H-imidazole-1-carboxamide hydrobromide

### Step1: tert-butyl 4-(4-(4-methoxyphenyl)-N-methyl-1H-imidazole-1-carboxamido)piperidine-1-carboxylate

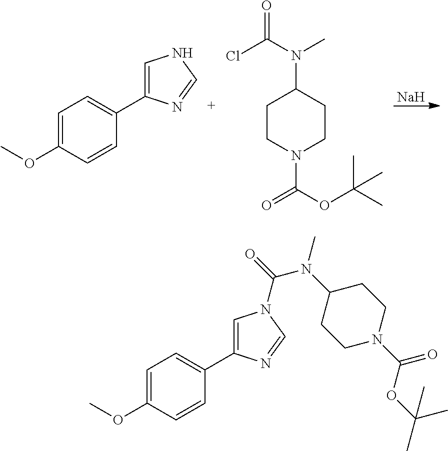

To a chilled suspension of 4-(4-methoxyphenyl)-1H-imidazole (intermediate 3) (8.03 g, 46.1 mmol) in Tetrahydrofuran (200 mL) was added sodium hydride (60% in mineral oil dispersion, 2.212 g, 55.3 mmol). After stirring the reaction mixture for ca 15 min, tert-butyl 4-(chlorocarbonyl(methyl)amino)piperidine-1-carboxylate (Intermediate 16) (14.03 g, 50.7 mmol) was added and stirring was continued at room temperature for 4 h. The reaction was quenched upon addition of water, transferred into a separatory funnel and partitioned between water and mixture of dichloromethane/isopropanol 7/3. The biphasic mixture was separated and the aqueous phase was further extracted into mixture of dichloromethane/isopropanol 7/3. The combined organic layers were dried over anhydrous Na₂SO₄, filtered through a short pad of silica/celite and concentrated. The residue was triturated with hot ethyl acetate, filtered and dried under vacuum. (Yield: 19 g, 99%).

### Step2: 4-(4-methoxyphenyl)-N-methyl-N-(piperidin-4-yl)-1H-imidazole-1-carboxamide hydrochloride

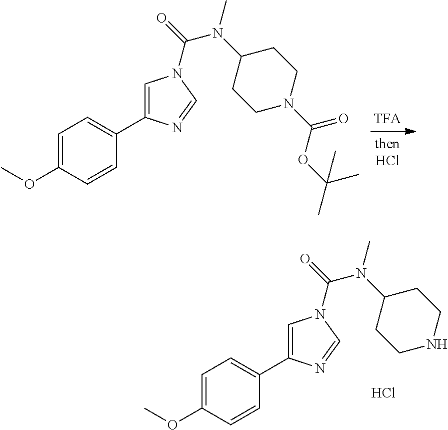

To a chilled portion of tert-butyl 4-(4-(4-methoxyphenyl)-N-methyl-1H-imidazole-1-carboxamido)piperidine-1-carboxylate (19 g, 45.8 mmol) was added trifluoro acetic acid (141 mL, 1834 mmol) and the reaction mixture was stirred in the cold for 1 h. Then, trifluoro acetic acid was removed under reduced pressure, the residue was dissolved in methanol and treated with excess of hydrogen chloride (2M solution in diethyl ether). The reaction was stirred until a thick white suspension was formed. Removal of the solvents under reduced pressure resulted in a sticky oil that was recrystallized from isopropanol. (Yield:17.52 g, 109%).

### Step3: 4-(4-hydroxyphenyl)-N-methyl-N-(piperidin-4-yl)-1H-imidazole-1-carboxamide hydrobromide

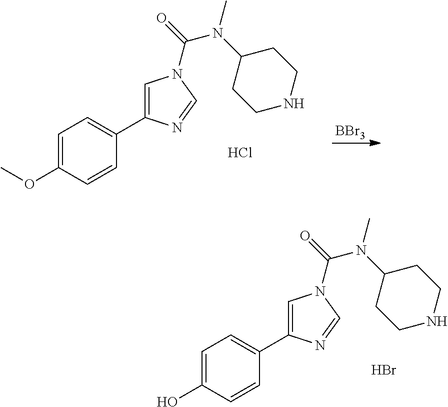

To a suspension of 4-(4-methoxyphenyl)-N-methyl-N-(piperidin-4-yl)-1H-imidazole-1-carboxamide hydrochloride (5 g, 14.25 mmol) in dry dichloromethane (190 mL) was added dropwise boron tribromide (5.39 mL, 57.0 mmol) at −78° C. The reaction was allowed to stir in the cold for 1 h, then at room temperature until its completion. Thereupon, the mixture was cooled to 0° C., carefully quenched with crushed ice and stirred for a while The obtained white solid was filtered off and dried under vacuum. The resulting crude mixture was separated by column chromatography (dichloromethane/methanol 95:5, then dichloromethane/methanol/25% aq. ammonia 7:1:0.2). (Yield: 4.91 g, 81%).

### Intermediate 13: 4-(3-hydroxyphenyl)-N-methyl-N-(piperidin-4-yl)-1H-imidazole-1-carboxamide hydrobromide

The title compound was prepared by analogous manner to intermediate 12 from Intermediate 2.

### Intermediate 14: 4-(3-aminophenyl)-N-(1-(benzo[d][1,3]dioxol-5-ylmethyl)piperidin-4-yl)-N-methyl-1H-imidazole-1-carboxamide

The title compound was prepared by analogous manner to intermediate 8 from benzo [d][1,3]dioxole-5-carbaldehyde.

### Intermediate 15: 4-(3-aminophenyl)-N-(1-(4-methoxyphenyl)piperidin-4-yl)-N-methyl-1H-imidazole-1-carboxamide

### Step1: N-(1-(4-methoxyphenyl)piperidin-4-yl)-N-methyl-4-(3-nitrophenyl)-1H-imidazole-1-carboxamide

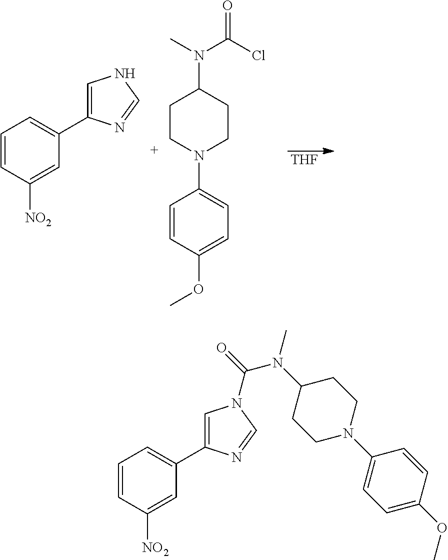

To a slightly turbid solution of Intermediate 1 (1.5 g, 7.93 mmol) in THF (39.6 mL) added sodium hydride (0.349 g, 8.72 mmol). After 15 min, Intermediate 6 (2.69 g, 9.52 mmol) was added and the reaction mixture was stirred at room temperature overnight. The resultant precipitate was filtered, washed with ether and dried under vacuum. (Yield: 3.43 g, 99%).

### Step2: 4-(3-aminophenyl)-N-(1-(4-methoxyphenyl)piperidin-4-yl)-N-methyl-1H-imidazole-1-carboxamide

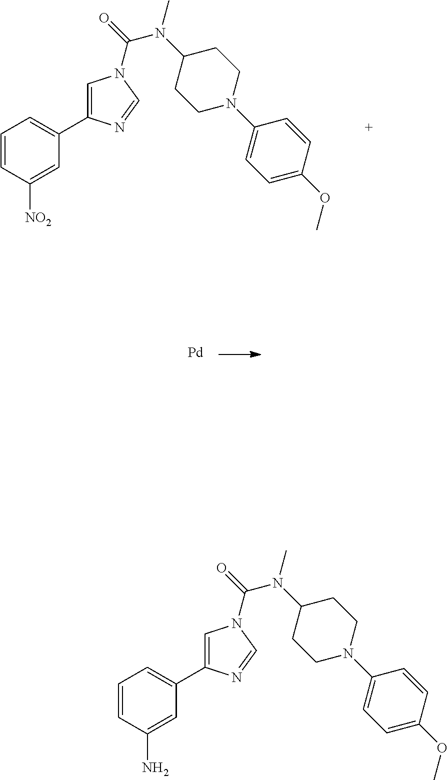

A suspension of N-(1-(4-methoxyphenyl)piperidin-4-yl)-N-methyl-4-(3-nitrophenyl)-1H-imidazole-1-carboxamide (2.5 g, 5.74 mmol) in ethanol (100 mL) and ethyl acetate (100 mL) was flushed with a stream of argon. Palladium (10% on charcoal, 0.305 g, 0.287 mmol) was added under inert atmosphere. The reaction mixture was stirred at room temperature under hydrogen atmosphere for 4 h. The suspension became a solution during the reaction course. Then, the reaction mixture was filtered through celite, concentrated and the resulting crude solid was recrystallized from hot isopropanol to yield the title compound as an off-white powder. (Yield: 745 mg, 32%).

### Intermediate 16: tert-butyl 4-((chlorocarbonyl)(methyl)amino)piperidine-1-carboxylate

### Step1: tert-butyl 4-(methylamino)piperidine-1-carboxylate

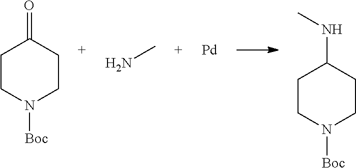

In a 250 mL flask was placed tert-butyl 4-oxopiperidine-1-carboxylate (20 g, 100 mmol) in methanol (100 mL), at room temperature under inert atmosphere, to give a colorless solution. Methanamine (40% in water) (38 mL, 441 mmol) was added, followed by palladium (10% on charcoal, 1.709 g, 1.606 mmol). The reaction flask was placed in an autoclave and was charged with 20 bar of hydrogen. The autoclave was heated at 50° C. and stirred for 2 h. The reaction mixture was cooled slowly, filtered through celite, and the solvent was evaporated. The crude product was dissolved in water and the aqueous mixture was extracted with ethyl acetate. The organics were dried over MgSO₄ and concentrated to yield a pale yellow oil. (Yield: 16.27 g, 72%).

### Step2: tert-butyl 4-((chlorocarbonyl)(methyl)amino)piperidine-1-carboxylate

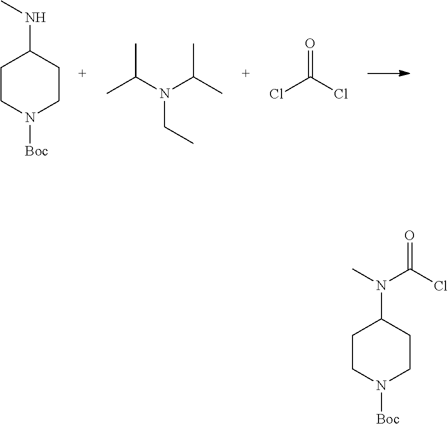

A solution of tert-butyl 4-(methylamino)piperidine-1-carboxylate (16.27 g, 76 mmol) and N,N-diisopropylethylamine (26.5 mL, 152 mmol) in tetrahydrofuran (84 mL) was added to phosgene (47.9 mL, 91 mmol) at 0° C. The reaction mixture was allowed to stir at room temperature for 3 h under inert atmosphere. The reaction mixture was quenched into ice and the solution was transferred to a separatory funnel and extracted with ethyl acetate. The organic layer was dried over MgSO₄ and concentrated under reduced pressure to yield a crude pale yellow solid that was triturated with heptane. (Yield: 11.22 g, 51%).

### Intermediate 17: 4-(4-(benzyloxy)phenyl)-1H-imidazole

### Step1: 1-(4-(benzyloxy)phenyl)ethanone

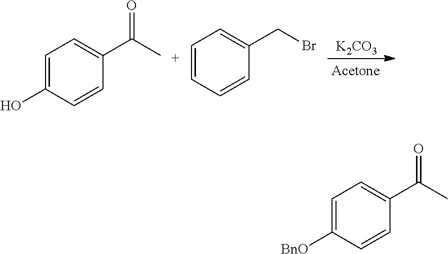

To a clear solution of 1-(4-hydroxyphenyl)ethanone (10 g, 73.4 mmol) in acetone (150 mL) was added potassium carbonate (13.20 g, 95 mmol), followed by benzyl bromide (11.42 mL, 95 mmol), dropwise. The mixture was heated at reflux overnight. Then, reaction mixture was cooled to room temperature, filtered, the filter cake was washed with acetone and the filtrate was evaporated. The resulting white solid was suspended in petroleum ether, filtered and dried to yield a white solid. (Yield: 16.22 g, 93%).

### Step2: 1-(4-(benzyloxy)phenyl)-2-bromoethanone

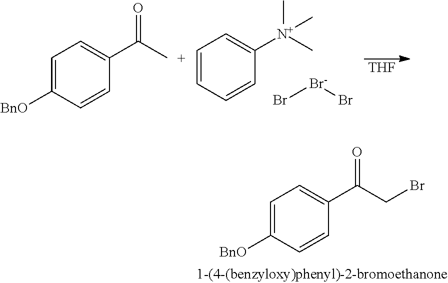

To a clear solution of 1-(4-(benzyloxy)phenyl)ethanone (16.1 g, 71.2 mmol) in tetrahydrofuran (200 mL) was added dropwise a solution of phenyltrimethylammonium tribromide (29.4 g, 78 mmol) in tetrahydrofuran (150 mL) at 0° C. After completion of the reaction, the insoluble material was filtered off and washed with tetrahydrofuran. The filtrate was evaporated and the yellow oil was crystallized from isopropanol. (Yield: 17.99 g, 83%).

### Step3: 4-(4-(benzyloxy)phenyl)-1H-imidazole

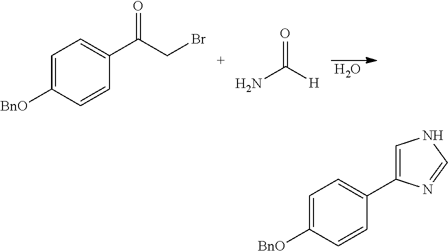

A mixture of 1-(4-(benzyloxy)phenyl)-2-bromoethanone (17.99 g, 59.0 mmol), formamide (29.1 mL, 731 mmol) and water (2 mL) was heated at 140° C. for 7 h. Then, it was poured onto 100 mL water/ice and the solid was filtered off. The filtrate was basified until pH 12, transferred into separatory funnel and extracted into dichloromethane/isopropanol 7:3; the organic layer was further washed with water, concentrated to afford a brown solid. (Yield: 8.36 g, 57%).

### Intermediate 18: (1-(4-bromo-3-methoxyphenyl)piperidin-4-yl)(methyl)carbamic chloride

### Step1 1-(4-bromo-3-methoxyphenyl)piperidin-4-one

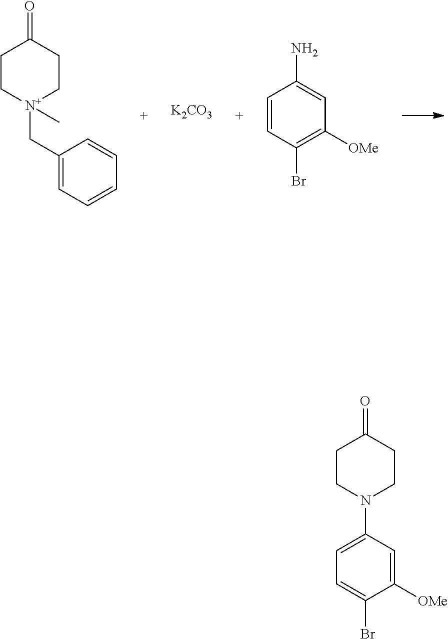 [SMILES: C[N+]1(Cc2ccccc2)CCC(=O)CC1 ; "K2CO3" ; COc1cc(N)ccc1Br ; COc1cc(N2CCC(=O)CC2)ccc1Br]

4-bromo-3-methoxyaniline (3 g, 14.85 mmol) was dissolved in ethanol (40 mL) and the resulting mixture was treated with potassium carbonate (0.287 g, 2.079 mmol). The reaction mixture was heated to reflux and a thick suspension of 1-benzyl-1-methyl-4-oxopiperidinium iodide (7.38 g, 22.27 mmol) in water (30 mL) was added portionwise. After 3 h of reflux, 50 mL of water were added to the reaction mixture and the solution was extracted with dichloromethane. The organic phase was dried over MgSO₄, concentrated and the crude product was purified by column chromatography (petroleum ether/ethyl acetate 4:1). The fractions containing the desired product were concentrated to yield a pale yellow oil. (Yield: 2.30 g, 54%).

### Step2 1-(4-bromo-3-methoxyphenyl)-N-methylpiperidin-4-amine

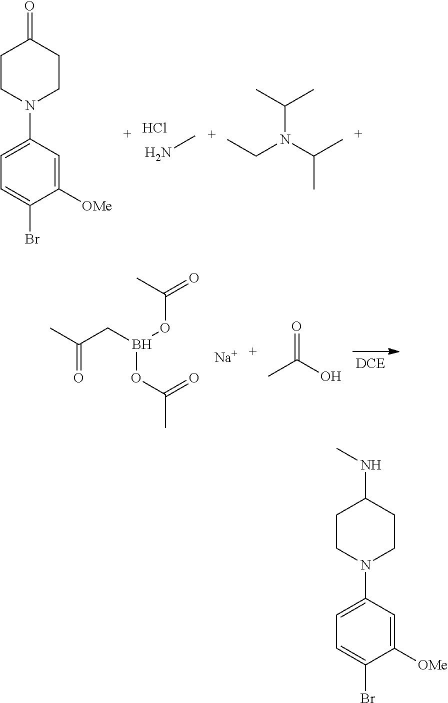

A suspension of methanamine hydrochloride (0.653 g, 9.67 mmol) in 1,2-dichloroethane (14 mL) was treated with N,N-diisopropylethylamine (5.62 mL, 32.2 mmol). The mixture turned homogeneous. Then 1-(4-bromo-3-methoxyphenyl)piperidin-4-one (2.29 g, 8.06 mmol) was added and the reaction was allowed to stir at room temperature. After 30 min, sodium triacetoxyhydroborate (3.42 g, 16.12 mmol) and acetic acid (0.461 mL, 8.06 mmol) were added to the reaction mixture and the reaction was stirred overnight. The reaction was quenched with ice and the heterogeneous mixture was transferred to a separatory funnel. The aqueous layer was extracted with dichloromethane/isopropanol 7:3. The organic layers were collected, dried over MgSO₄, and concentrated under vacuum. The crude mixture was purified by column chromatography (dichloromethane/methanol 9:1) to yield a yellow oil. (Yield: 1.736 g, 72%).

### Step3 (1-(4-bromo-3-methoxyphenyl)piperidin-4-yl)(methyl)carbamic chloride

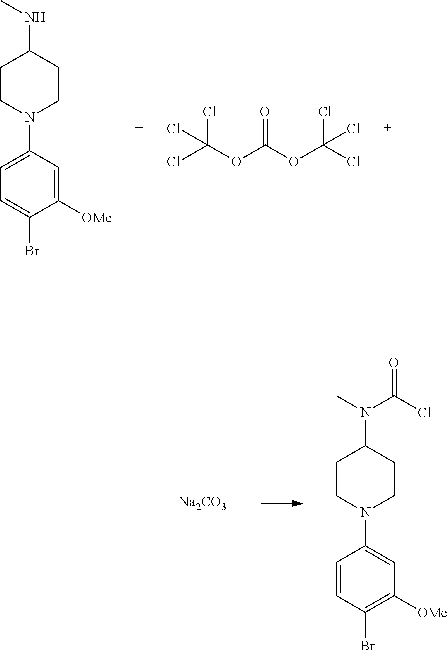 [SMILES: CNC1CCN(c2ccc(Br)c(OC)c2)CC1 ; O=C(OC(Cl)(Cl)Cl)OC(Cl)(Cl)Cl ; "Na2CO3" ; COc1cc(N2CCC(N(C)C(=O)Cl)CC2)ccc1Br]

A cold solution of bis(trichloromethyl) carbonate (0.686 g, 2.313 mmol) in dichloromethane (12 mL) was treated with a solution of 1-(4-bromo-3-methoxyphenyl)-N-methylpiperidin-4-amine (1.73 g, 5.78 mmol) in dichloromethane (12 mL). Then, Sodium carbonate (1.226 g, 11.56 mmol) was added portionwise (gas evolution). The reaction was allowed to warm up to room temperature and was stirred for 3 h. The solvent was removed under vacuum and the obtained crude product was purified by column chromatography (petroleum ether/ethyl acetate 4:1) to yield the title product as an off-white solid. (Yield: 1.762 g, 84%).

### Intermediate 19: 3-(1H-imidazol-4-yl)benzamide

### Step1 3-(1H-imidazol-4-yl)benzonitrile

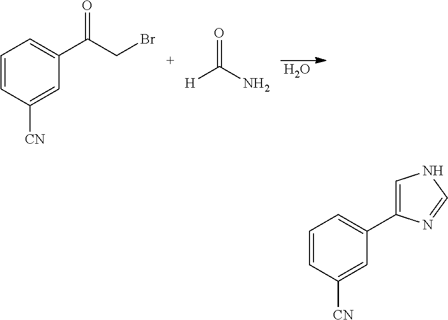

In a 100 mL round-bottomed flask 3-(2-bromoacetyl)benzonitrile (10 g, 44.6 mmol), formamide (21.98 mL, 553 mmol) and water (1.65 mL) were placed. The mixture was heated at 140° C. and stirred for 2 h. Then, it was cooled to room temperature and poured into 1N HCl solution (100 mL). The resultant precipitate was filtered off and washed with 1N HCl solution. The filtrate's pH was set to 10 by adding 3N NaOH solution. The resultant precipitate was filtered off, washed with water and dried under vacuum. (Yield: 1.95 g, 26%).

### Step2: 3-(1H-imidazol-4-yl)benzamide

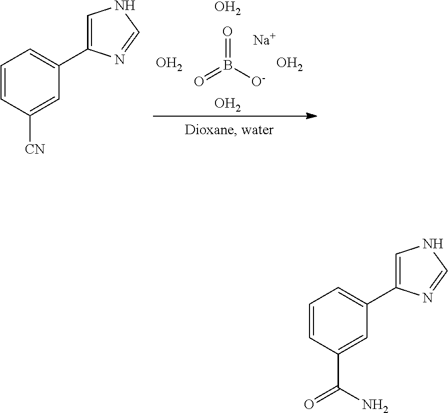

In a 100 mL round-bottomed flask were placed 3-(1H-imidazol-4-yl)benzonitrile (521.6 mg, 3.08 mmol), water (10 mL) and dioxane (10 mL). Then, sodium perborate tetrahydrate (1309 mg, 8.51 mmol) was added and the mixture was heated at 80° C. for 48 h. A second crop of sodium perborate tetrahydrate (750 mg, 4.87 mmol) was added and the mixture was stirred at 80° C. for further 24 h. Then the reaction mixture was cooled to room temperature and dioxane was removed under reduced pressure. The aqueous phase was extracted several times with a mixture of dichloromethane/isopropanol 7:3. The combined organic layers were dried over MgSO₄ and concentrated. The resultant foam was dissolved in minimal volume of methanol and precipitated upon addition of diethyl ether. The precipipate was filtered and dried under vacuum. (Yield: 380.7 mg, 59%).

### Intermediate 20: 4-(3-carbamoylphenyl)-N-methyl-N-(piperidin-4-yl)-1H-imidazole-1-carboxamide hydrochloride

### Step1: tert-butyl 4-(4-(3-carbamoylphenyl)-N-methyl-1H-imidazole-1-carboxamido)piperidine-1-carboxylate

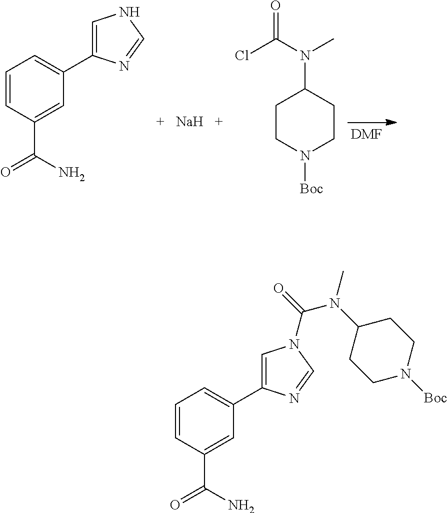

In a 50 mL round-bottomed flask, 3-(1H-imidazol-4-yl)benzamide (0.75 g, 4.01 mmol) (intermediate 19) and N,N-dimethylformamide (20 mL) were placed under inert atmosphere. The mixture was cooled to 0° C. and sodium hydride (60% in mineral oil, 0.192 g, 4.81 mmol) was added portionwise. Then, the reaction mixture was allowed to warm up to room temperature and stirred for 30 min. Thereupon, tert-butyl 4-(chlorocarbonyl(methyl)amino)piperidine-1-carboxylate (1.331 g, 4.81 mmol) (intermediate 16) was added portionwise and the reaction mixture was stirred for 2.5 h. Then the mixture was cooled to 0° C. and quenched with water. The two phases were separated and the aqueous phase was extracted several times with dichloromethane/isopropanol 7:3. The combined organic layers were dried over MgSO₄ and concentrated. The obtained residue was purified by column chromatography (dichloromethane/methanol 1:0, 9:1) to yield the title product as a colourless oil which was further triturated with ethyl ether to give a solid. (Yield: 0.656 g, 38%).

### Step2: 4-(3-carbamoylphenyl)-N-methyl-N-(piperidin-4-yl)-1H-imidazole-1-carboxamide hydrochloride

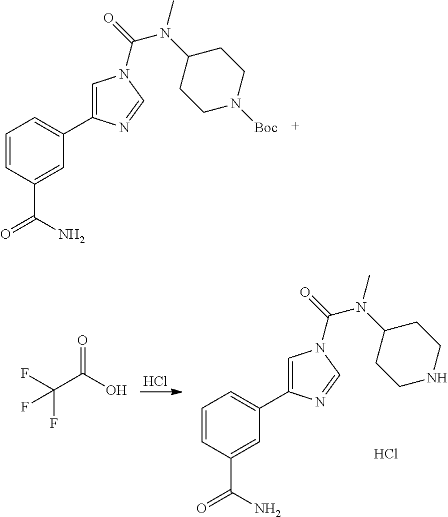 [SMILES: CN(C(=O)n1cnc(-c2cccc(C(N)=O)c2)c1)C1CCN(C(=O)OC(C)(C)C)CC1 ; O=C(O)C(F)(F)F ; Cl ; CN(C(=O)n1cnc(-c2cccc(C(N)=O)c2)c1)C1CCNCC1 ; Cl]

tert-butyl 4-(4-(3-carbamoylphenyl)-N-methyl-1H-imidazole-1-carboxamido)piperidine-1-carboxylate (500 mg, 1.170 mmol) was dissolved in trifluoroacetic acid (2 mL), at 0° C., and the reaction was allowed to stir vigorously at room temperature for 1 h. Thereupon, trifluoroacetic acid was removed under reduced pressure and the obtained residue was dissolved in 5 mL of ethyl acetate. The solution was cooled to 0° C. and treated with 2N hydrogen chloride solution in diethyl ether (0.585 mL, 1.17 mmol). The precipitate was filtered, washed with diethyl ether and dried under vacuum. (Yield: 380 mg, 89%).

### Intermediate 21: 4-(3-carbamoylphenyl)-N-(1-(2-methoxybenzyl)piperidin-4-yl)-N-methyl-1H-imidazole-1-carboxamide

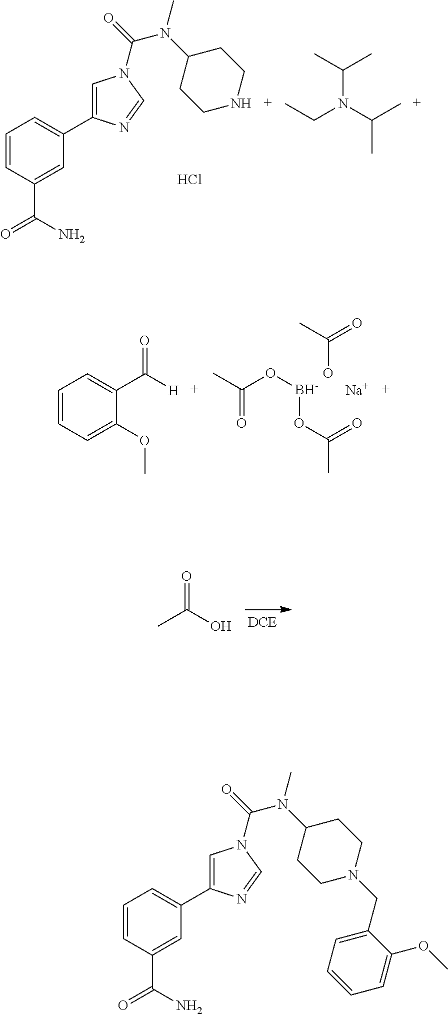

A suspension of 4-(3-carbamoylphenyl)-N-methyl-N-(piperidin-4-yl)-1H-imidazole-1-carboxamide hydrochloride (0.5 g, 1.374 mmol) (Intermediate 20), in 1,2-Dichloroethane (16 mL) was treated with N-ethyl-N-isopropylpropan-2-amine (0.96 mL, 5.50 mmol). The mixture turned homogeneous. Then 2-methoxybenzaldehyde (0.374 g, 2.75 mmol) was added and the reaction was allowed to stir at room temperature. After 30 min., sodium triacetoxyhydroborate (0.583 g, 2.75 mmol) was added followed by acetic acid (0.08 mL, 1.374 mmol) and the reaction was stirred overnight. Thereupon, the reaction was quenched with crushed ice and the heterogeneous mixture was transferred to a separatory funnel. The aqueous layer was extracted with a mixture of DCM:IPA 7/3. The organic layers were combined, dried over MgSO₄, and concentrated under vacuum. The crude mixture was purified by column chromatography (DCM to DCM:MeOH 10%), then precipitated from diethyl ether to yield intermediate 21 (286 mg, 56% yield) as an off-white powder.

### Example 1

### N-(1-benzylpiperidin-4-yl)-N-methyl-4-(3-(methylsulfonamido)phenyl)-1H-imidazole-1-carboxamide hydrochloride

### Step1: N-(1-benzylpiperidin-4-yl)-N-methyl-4-(3-(methylsulfonamido)phenyl)-1H-imidazole-1-carboxamide

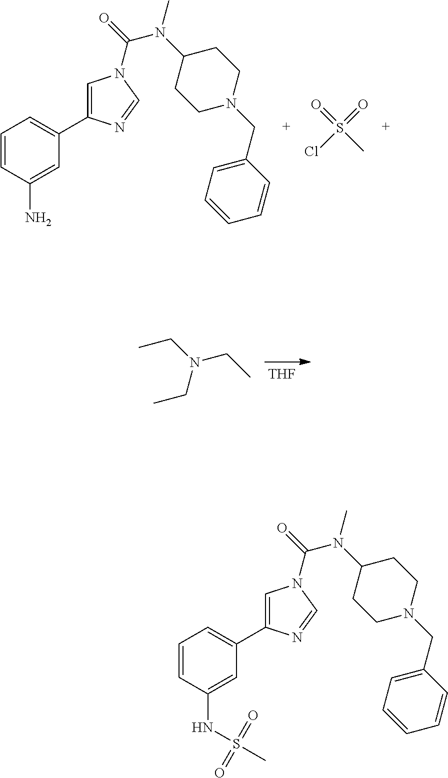

Methanesulfonyl chloride (0.220 mL, 2.82 mmol) was added to a stirred suspension of 4-(3-aminophenyl)-N-(1-benzylpiperidin-4-yl)-N-methyl-1H-imidazole-1-carboxamide (Intermediate 7) (1 g, 2.57 mmol) and triethylamine (0.391 mL, 2.82 mmol) in tetrahydrofuran (5 mL) at room temperature. The mixture was allowed to stir at room temperature overnight. The solvent was removed under reduced pressure. The residue was dissolved in a mixture of dichloromethane/isopropanol 7:3 and washed with water. The aqueous layer was extracted with a mixture of dichloromethane/isopropanol 7:3. The combined organic layers were dried over MgSO₄ and evaporated to give a colorless oil. The product was separated by column chromatography (dichloromethane/methanol 9:1) and was precipitated by trituration with petroleum ether. (Yield: 0.227 g, 19%).

### Step2 N-(1-benzylpiperidin-4-yl)-N-methyl-4-(3-(methylsulfonamido)phenyl)-1H-imidazole-1-carboxamide hydrochloride

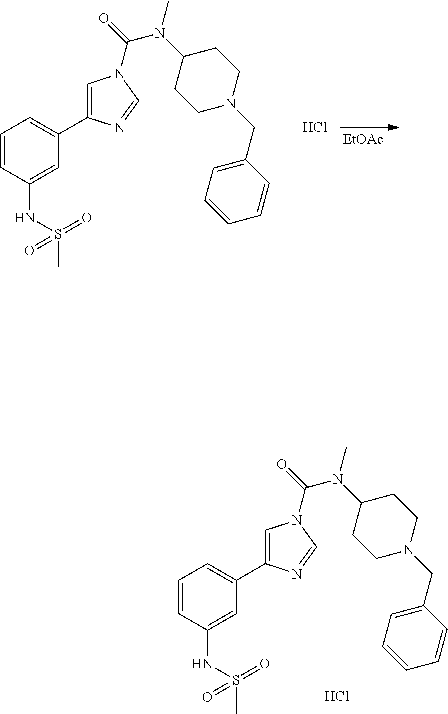 [SMILES: "EtOAc" ; CN(C(=O)n1cnc(-c2cccc(NS(C)(=O)=O)c2)c1)C1CCN(Cc2ccccc2)CC1 ; Cl ; CN(C(=O)n1cnc(-c2cccc(NS(C)(=O)=O)c2)c1)C1CCN(Cc2ccccc2)CC1 ; Cl]

In a 50 mL pear flask N-(1-benzylpiperidin-4-yl)-N-methyl-4-(3-(methylsulfonamido)phenyl)-1H-imidazole-1-carboxamide (420 mg, 0.898 mmol) and ethyl acetate (3 ml) were placed. The mixture was cooled to 0° C. and treated dropwise with HCl 2N diethyl ether solution (3.37 ml, 6.74 mmol). The resultant white suspension was stirred at 0° C. for 10 min, then was allowed to warm up to room temperature and stirred for 2 h. The precipitate was filtered; the filter cake was washed with diethyl ether and dried under vacuum. (Yield: 0.39 g, 86%).

¹H NMR (DMSO), δ(ppm): 10.77 (1H, s br), 9.82 (1H, s), 8.34 (1H, s br), 8.04 (1H, s), 7.72 (1H, t, J=2 Hz), 7.60 (2H, m), 7.57 (1H, ddd, J=1.0, 1.5, 8.0 Hz), 7.47 (3H, m), 7.36 (1H, t, J=7.8 Hz), 7.13 (1H, ddd, J=1.0, 2.0, 8.0 Hz), 4.26 (2H, d, J=5.2 Hz), 4.18 (1H, br), 3.40 (2H, d, J=12.0 Hz), 3.10 (2H, mq, J=12.3 Hz), 3.10 (2H, mq, J=12.0 Hz), 3.0 (3H, s), 2.93 (3H, s), 2.31 (2 h, dq, J=3.5, 13.5 Hz), 1.96 (2H, d, J=13.0 Hz).

¹³C NMR (DMSO), δ (ppm): 150.4, 138.9, 138.4, 137.7, 132.7, 131.4, 129.8, 129.7, 129.4, 128.8, 120.8, 119.3, 116.5, 115.3, 58.7, 52.2, 50.2, 39.8, 31.5, 24.7.

### Example 2

### N-(1-benzylpiperidin-4-yl)-N-methyl-4-(3-(sulfamoylamino)phenyl)-1H-imidazole-1-carboxamide hydrochloride

### Step1: N-(1-benzylpiperidin-4-yl)-N-methyl-4-(3-(sulfamoylamino)phenyl)-1H-imidazole-1-carboxamide

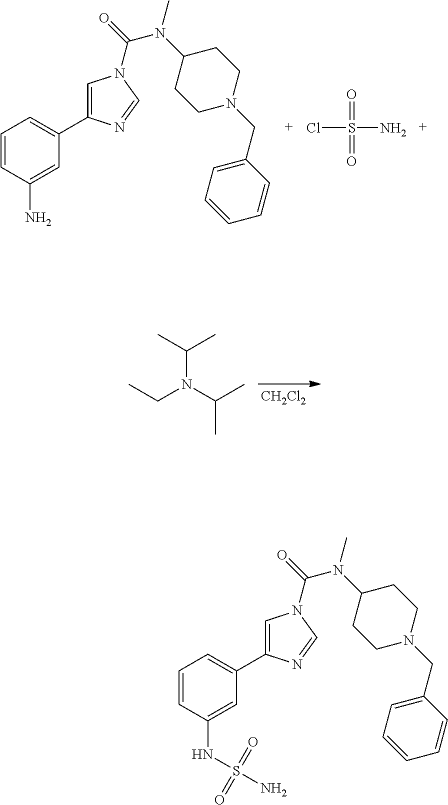

In a 50 mL pear flask 4-(3-aminophenyl)-N-(1-benzylpiperidin-4-yl)-N-methyl-1H-imidazole-1-carboxamide (Intermediate 7) (0.60 g, 1.540 mmol), anhydrous dichloromethane (20 mL) and N,N-diisopropylethylamine (0.404 ml, 2.311 mmol) were placed. The reaction mixture was cooled to 0° C. and sulfamoyl chloride (0.214 g, 1.849 mmol) was added. The reaction mixture was stirred at 0° C. for 10 min and then was allowed to warm up to room temperature and stirred for 24 h. Then, another portion of sulfamoyl chloride (0.214 mg, 1.849 mmol) was added and the reaction was stirred for 1 h. The reaction mixture was filtered and washed with dichloromethane. The filtered cake was suspended in 500 mL of hot mixture of dichloromethane/isopropanol, then the solvents were evaporated to small volume. The precipitate was filtered and purified by column chromatography (dichloromethane/methanol, 49:1, 19:1, 9:1, 5:1). (Yield: 0.100 g, 12%).

### Step2: N-(1-benzylpiperidin-4-yl)-N-methyl-4-(3-(sulfamoylamino)phenyl)-1H-imidazole-1-carboxamide hydrochloride

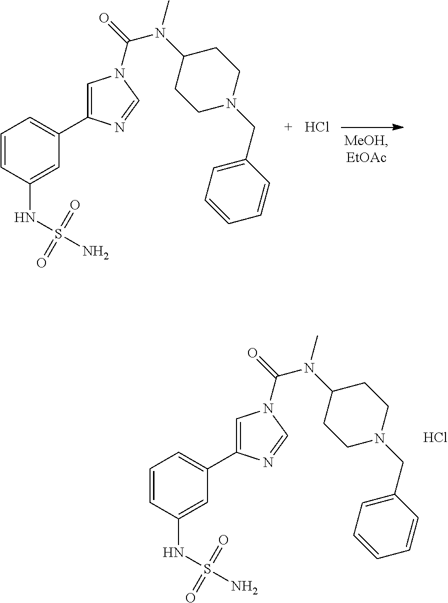 [SMILES: CN(C(=O)n1cnc(-c2cccc(NS(N)(=O)=O)c2)c1)C1CCN(Cc2ccccc2)CC1 ; Cl ; CN(C(=O)n1cnc(-c2cccc(NS(N)(=O)=O)c2)c1)C1CCN(Cc2ccccc2)CC1 ; Cl]

In a 50 mL pear flask N-(1-benzylpiperidin-4-yl)-N-methyl-4-(3-(sulfamoylamino)phenyl)-1H-imidazole-1-carboxamide (100 mg, 0.213 mmol), ethyl acetate (2 mL) and methanol (2 mL) were placed. The mixture was cooled to 0° C. and treated dropwise with HCl 2N diethyl ether solution (0.80 ml, 1.601 mmol). The resultant white suspension was stirred at 0° C. for 10 min, then was allowed to warm to room temperature and stirred for 2 h. The precipitate was filtered off; the filter cake was washed with diethyl ether and dried under vacuum. (Yield: 87 mg, 81%).

¹H NMR (DMSO), δ (ppm): 11.26 (1H, s br), 9.66 (1H, s), 8.77 (1H, s br), 8.15 (1H, s), 7.63 (3H, m), 7.47 (4H, m), 7.33 (1H, t, J=8.0 Hz), 7.24 (2H, br), 7.09 (1H, dd, J=1.5, 8.0 Hz), 4.26 (2H, d, J=4.8 Hz), 4.20 (1H, s br), 3.39 (2H, d, J=11.5 Hz), 3.09 (2H, q, J=11.5 Hz), 2.96 (3H, s), 2.40 (2H, dq, J=3.0, 12.5 Hz), 1.96 (2H, d, J=12.5 Hz).

¹³C NMR (DMSO), δ (ppm): 150, 140.1, 137.7, 137.4, 131.4, 131.2, 129.8, 129.4, 129.3, 128.8, 118.9, 117.8, 115.4, 114.3, 58.7, 52.3, 50.2, 31.6, 24.7

### Example 3

### N-(1-(3-methoxybenzyl)piperidin-4-yl)-N-methyl-4-(3-(sulfamoylamino)phenyl)-1H-imidazole-1-carboxamide hydrochloride

### Step1: N-(1-(3-methoxybenzyl)piperidin-4-yl)-N-methyl-4-(3-(sulfamoylamino)phenyl)-1H-imidazole-1-carboxamide

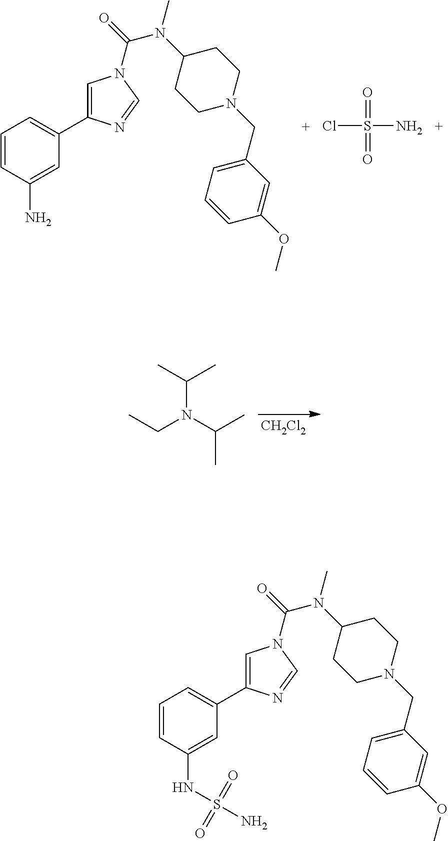

In a 50 mL pear flask 4-(3-aminophenyl)-N-(1-(3-methoxybenzyl)piperidin-4-yl)-N-methyl-1H-imidazole-1-carboxamide (Intermediate 8) (0.420 g, 1.001 mmol), anhydrous dichloromethane (15 ml) and N,N-diisopropylethylamine (0.262 ml, 1.502 mmol) were placed. The reaction mixture was cooled to 0° C. and sulfamoyl chloride (0.139 g, 1.849 mmol) was added. The reaction mixture was stirred at 0° C. for 10 min, then was allowed to warm to room temperature and stirred overnight. The reaction mixture was quenched with water. The phases were separated and the organic layer was dried over MgSO₄, filtered, concentrated and purified by column chromatography (dichloromethane/methanol 49:1, 19:1, 9:1). (Yield: 0.428 g, 77%).

### Step2: N-(1-(3-methoxybenzyl)piperidin-4-yl)-N-methyl-4-(3-(sulfamoylamino)phenyl)-1H-imidazole-1-carboxamide hydrochliride

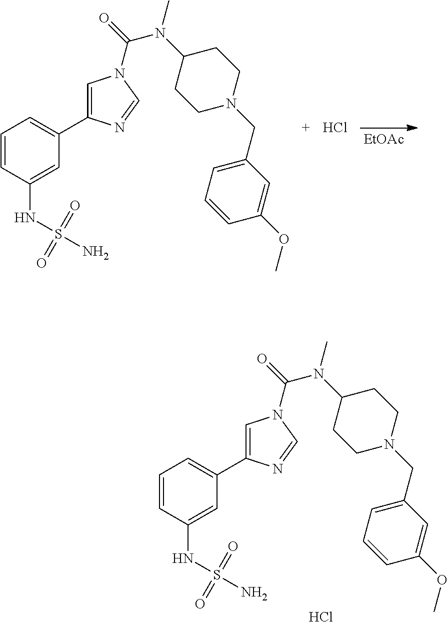 [SMILES: "EtOAc" ; COc1cccc(CN2CCC(N(C)C(=O)n3cnc(-c4cccc(NS(N)(=O)=O)c4)c3)CC2)c1 ; Cl ; COc1cccc(CN2CCC(N(C)C(=O)n3cnc(-c4cccc(NS(N)(=O)=O)c4)c3)CC2)c1 ; Cl]

In a 50 mL pear flask N-(1-(3-methoxybenzyl)piperidin-4-yl)-N-methyl-4-(3-(sulfamoylamino)phenyl)-1H-imidazole-1-carboxamide (428 mg, 0.858 mmol) and ethyl acetate (5 mL) were placed. The mixture was cooled to 0° C. and treated dropwise with HCl 2N diethyl ether solution (3.22 ml, 6.44 mmol). The resultant white suspension was stirred at 0° C. for 10 min, then was allowed to warm up to room temperature and stirred for 2 h. The precipitate was filtered off, washed with diethyl ether and recrystallized from a hot mixture of dichloromethane/methanol. (Yield: 93 mg, 20%).

¹H NMR (DMSO), δ (ppm): 11.31 (1H, s br), 9.67 (1H, s br), 8.80 (1H, s br), 8.16 (1H, s), 7.63 (1H, t, J=1.9 Hz), 7.47 (1H, tt, J=1.2, 7.8 Hz), 7.38-7.31 (3H, m), 7.24 (2H, br), 7.14 (1H, d, J=7.5 Hz), 7.10 (1H, ddd, J=1.0, 2.0, 8.0 Hz), 7.0 (1H, dd, J=3.0, 8.3 Hz), 4.22 (2H, d, J=5.1 Hz), 4.20 (1H, br), 3.79 (3H, s), 3.38 (2H, d, J=11.5 Hz), 3.08 (2H, q, J=11.0 Hz), 2.97 (3H, s), 2.43 (2H, dq, J=3.3, 12.7 Hz), 1.96 (2H, d, J=12.3 Hz).

¹³C NMR (DMSO), δ (ppm): 159.4, 149.9, 140.1, 137.7, 137.4, 131.3, 131.1, 129.9, 129.3, 123.3, 118.9, 117.8, 116.6, 115.4, 115.2, 114.3, 58.8, 55.2, 52.3, 50.3, 31.5, 24.6.

### Example 4

### N-(1-(3-methoxybenzyl)piperidin-4-yl)-N-methyl-4-(3-(methylsulfonamido)phenyl)-1H-imidazole-1-carboxamide hydrochloride

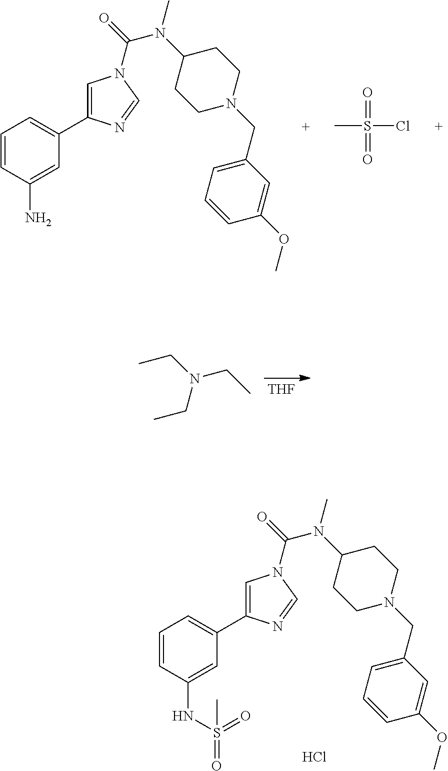

Methanesulfonyl chloride (0.085 mL, 1.101 mmol) was added to a stirred solution of 4-(3-aminophenyl)-N-(1-(3-methoxybenzyl)piperidin-4-yl)-N-methyl-1H-imidazole-1-carboxamide (Intermediate 8) (420 mg, 1.001 mmol) and triethylamine (0.153 mL, 1.101 mmol) in tetrahydrofuran (15 mL). The mixture was stirred overnight at room temperature. Then, the resultant precipitate was filtered off and washed with tetrahydrofuran. The filter cake was dissolved in a mixture of dichloromethane/isopropanol 7:3 and washed with water. The organic layer was dried over MgSO₄ and the solvent was evaporated to yield a pale yellow solid that was triturated with diethyl ether, filtered and dried under vacuum. (Yield: 87 mg, 15%).

¹H NMR (DMSO), δ (ppm): 11.85 (1H, s br), 9.78 (1H, s), 8.14 (1H, s), 7.97 (1H, s), 7.74 (1H, s), 7.56 (1H, d, J=7.7 Hz), 7.74-7.19 (2H, m br), 7.34 (1H, t, J=7.8 Hz), 7.11 (1H, dd, J=2.5, 7.9 Hz), 7.07-6.70 (2H, m br), 4.20 (3H, m), 3.78 (3H, s), 3.37 (2H, m), 3.07 (2H, m), 2.99 (3H, s), 2.94 (3H, s), 2.34 (2H, m), 1.96 (2H, m).

¹³C NMR (DMSO), δ (ppm): 159, 150.6, 139.9, 138.4, 137.3, 134.1, 130.9, 129.5, 129.2, 122.9, 120.1, 118.2, 116.3, 115.9, 114.7, 114.4, 58.5, 54.8, 51.9, 50.2, 31.2, 24.5.

### Example 5

### N-(1-(3,4-dimethoxybenzyl)piperidin-4-yl)-N-methyl-4-(3-(methylsulfonamido)phenyl)-1H-imidazole-1-carboxamide hydrochloride

### Step1: N-(1-(3-methoxybenzyl)piperidin-4-yl)-N-methyl-4-(3-(sulfamoylamino)phenyl)-1H-imidazole-1-carboxamid

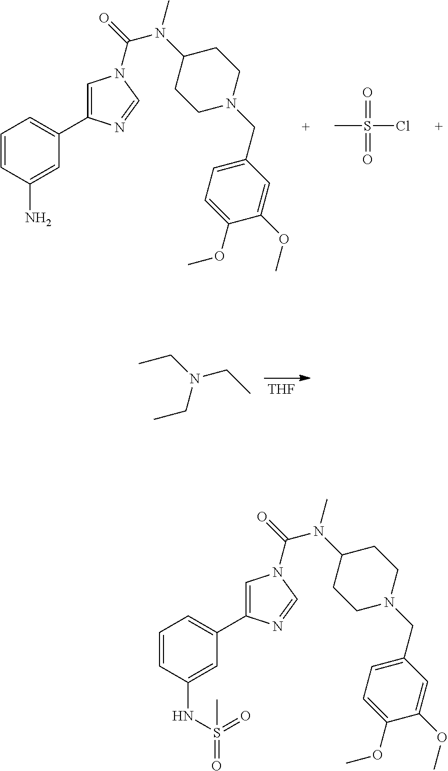

In a 50 mL pear flask 4-(3-aminophenyl)-N-(1-(3,4-dimethoxybenzyl)piperidin-4-yl)-N-methyl-1H-imidazole-1-carboxamide (Intermediate 9) (0.700 g, 1.557 mmol), anhydrous tetrahydrofuran (25 ml) and triethylamine (0.237 ml, 1.713 mmol) were placed. The reaction mixture was cooled to 0° C. and sulfamoyl chloride (0.133 mL, 1.713 mmol) was added. The reaction mixture was stirred at room temperature overnight. Then, another portion of triethylamine (0.237 ml, 1.713 mmol) was added followed by sulfamoyl chloride (0.67 mL, 0.86 mmol) and the reaction was stirred for 3 h. The solvent was removed under vacuum; the residue was dissolved in a mixture of dichloromethane/isopropanol 7/3 and washed with water. The organic layer was dried over MgSO₄ and the solvent was evaporated. The obtained foam was purified by column chromatography (dichloromethane/methanol 98:2, 95:5, 9:1). (Yield: 0.650 g, 75%).

### Step2: N-(1-(3, 4-dimethoxybenzyl)piperidin-4-yl)-N-methyl-4-(3-(methylsulfonamido)phenyl)-1H-imidazole-1-carboxamide hydrochloride

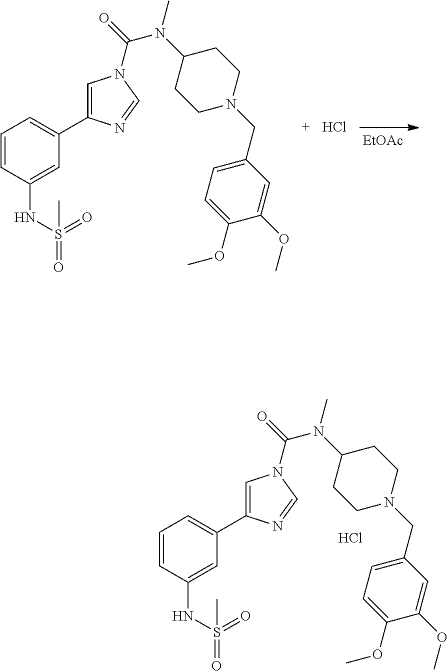 [SMILES: "EtOAc" ; COc1ccc(CN2CCC(N(C)C(=O)n3cnc(-c4cccc(NS(C)(=O)=O)c4)c3)CC2)cc1OC ; Cl ; COc1ccc(CN2CCC(N(C)C(=O)n3cnc(-c4cccc(NS(C)(=O)=O)c4)c3)CC2)cc1OC ; Cl]

In a 50 mL pear flask N-(1-(3,4-dimethoxybenzyl)piperidin-4-yl)-N-methyl-4-(3-(methylsulfonamido)phenyl)-1H-imidazole-1-carboxamide (650 mg, 1.232 mmol) and ethyl acetate (10 mL) were placed. The mixture was cooled to 0° C. and treated dropwise with HCl 2N diethyl ether solution (4.62 ml, 9.24 mmol). The resultant yellow solution was stirred at 0° C. for 10 min, then was allowed to warm up to room temperature and stirred for 2 h. The resultant precipitate was filtered off, washed with diethyl ether and recrystallized from a hot mixture of dichloromethane/methanol. (Yield: 538 mg, 69%).

¹H NMR (DMSO), δ (ppm): 11.20 (1H, s br), 9.88 (1H, s), 8.63 (1H, s), 8.13 (1H, s), 7.72 (1H, t, J=1.8 Hz), 7.59 (1H, ddd, J=1.0, 1.5, 7.8 Hz), 7.41 (1H, d, J=1.9 Hz), 7.39 (1H, t, J=7.9 Hz), 7.16 (1H, ddd, J=1.0, 2.2, 8.1 Hz), 7.04 (1H, dd, J=1.8, 8.2 Hz), 6.98 (1H, d, J=8.2 Hz), 4.20 (1H, s br), 4.17 (2H, d, J=5.2 Hz), 3.80 (3H, s), 3.77 (3H, s), 3.36 (2H, d, J=11.0 Hz), 3.05 (2H, m), 3.03 (3H, s), 2.95 (3H, s), 2.41 (2H, dq, J=3.5, 12.5 Hz), 1.95 (2H, d, J=12.5 Hz).

¹³C NMR (DMSO), δ (ppm): 150.2, 149.5, 148.6, 138.9, 138, 137.7, 132.3, 129.8, 123.8, 121.9, 120.9, 119.5, 116.5, 115.4, 114.6, 111.4, 58.8, 55.6, 55.5, 52.3, 50, 31.5, 24.7.

### Example 6

### 3-(1-(methyl(1-phenylpiperidin-4-yl)carbamoyl)-1H-imidazol-4-yl)phenyl sulfamate hydrochloride

### Step1: 3-(1-(methyl(1-phenylpiperidin-4-yl)carbamoyl)-1H-imidazol-4-yl)phenyl sulfamate

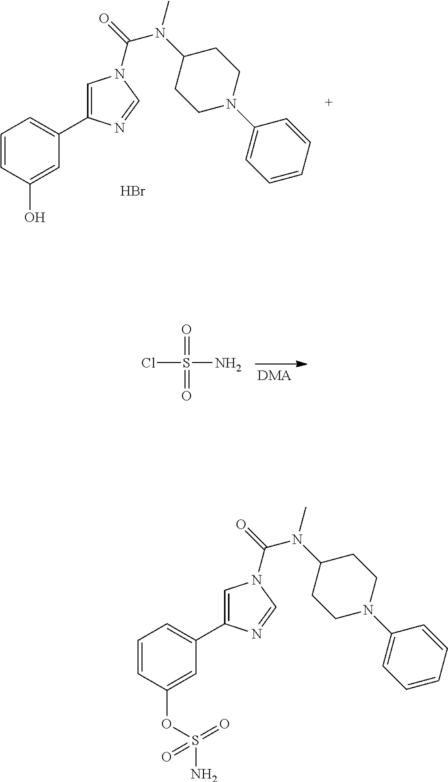

In a 25 mL round-bottomed flask, 4-(3-hydroxyphenyl)-N-methyl-N-(1-phenylpiperidin-4-yl)-1H-imidazole-1-carboxamide hydrobromide (Intermediate 10) (200 mg, 0.437 mmol) and N,N-dimethylacetamide (2 mL) were placed under inert atmosphere. Sulfamoyl chloride (101 mg, 0.875 mmol) was added and the solution was stirred at room temperature for 24 h. Then, another portion of sulfamoyl chloride (101 mg, 0.875 mmol) was added and the solution was stirred for further 2 h. The reaction mixture was quenched with water, followed by addition of pyridine. The resultant precipitate was filtered, washed with water and recrystallized from isopropanol. (Yield: 105 mg, 47%).

### Step2: 3-(1-(methyl(1-phenylpiperidin-4-yl)carbamoyl)-1H-imidazol-4-yl)phenyl sulfamate hydrochloride

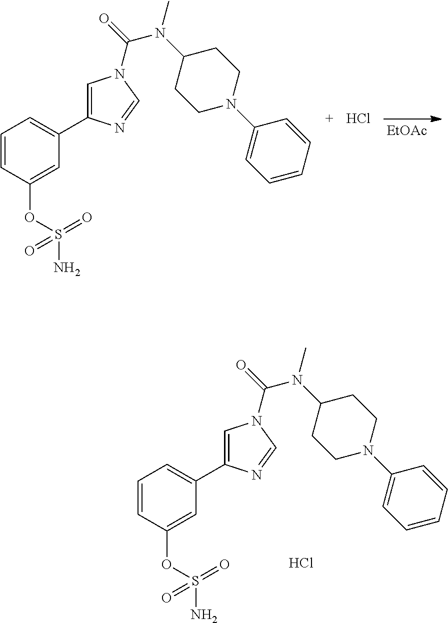 [SMILES: "EtOAc" ; CN(C(=O)n1cnc(-c2cccc(OS(N)(=O)=O)c2)c1)C1CCN(c2ccccc2)CC1 ; Cl ; CN(C(=O)n1cnc(-c2cccc(OS(N)(=O)=O)c2)c1)C1CCN(c2ccccc2)CC1 ; Cl]

In a 25 mL pear flask were placed 3-(1-(methyl(1-phenylpiperidin-4-yl)carbamoyl)-1H-imidazol-4-yl)phenyl sulfamate (105 mg, 0.231 mmol) and ethyl acetate (4 mL). The mixture was cooled to 0° C. and treated dropwise with HCl 2N diethyl ether solution (0.864 mL, 1.729 mmol). The white suspension was stirred at 0° C. for 10 min, then was allowed to warm up to room temperature and stirred for 2 h. The precipitate was filtered off, the filter cake was washed with diethyl ether and dried under vacuum. (Yield: 90 mg, 79%).

¹H NMR (DMSO), δ (ppm): 13.43 (1H, br), 8.70 (1H, s br), 8.30 (1H, s), 8.10 (2H, s), 7.88 (2H, s br), 7.85 (1H, td, J=1.0, 8.0 Hz), 7.82 (1H, t, J=1.9 Hz), 7.56 (2H, t br, J=7.0 Hz, 7.53 (1H, t, J=8.0 Hz), 7.47 (1H, s br), 7.25 (1H, ddd, J=0.8, 2.5, 8.0 Hz), 4.47 (1H, s br), 3.78 (2H, s br), 3.64 (2H, d, J=10.5 Hz), 3.04 (3H, s), 2.71 (2H, s br), 2.05 (2H, d, J=12.5 Hz).

¹³C NMR (DMSO), δ (ppm): 150.7, 150.2, 143.2, 137.9, 137.3, 133, 130.2, 130, 123.2, 121.4, 121.1, 118.7, 116, 54, 51.8, 31.6, 25.4.

### Example 7

### 3-(1-((1-benzylpiperidin-4-yl)(methyl)carbamoyl)-1H-imidazol-4-yl)phenyl sulfamate hydrochloride

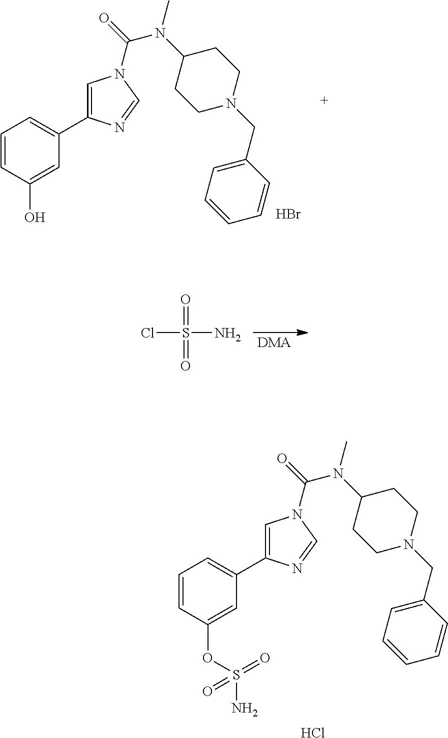

In a 25 mL pear flask, N-(1-benzylpiperidin-4-yl)-4-(3-hydroxyphenyl)-N-methyl-1H-imidazole-1-carboxamide hydrobromide (162 mg, 0.345 mmol) and N,N-dimethylacetamide (1.5 mL) were placed, under inert atmosphere. Sulfamoyl chloride (96 mg, 0.830 mmol) was added and the solution was stirred at room temperature for 24 h. Another portion of sulfamoyl chloride (96 mg, 0.830 mmol) was added and the solution was stirred for additional 2 h. Then, the reaction was quenched with water, followed by addition of pyridine. The solvent was evaporated with toluene and the crude oil was purified by column chromatography (dichloromethane/methanol, 49:1, 19:1, 9:1, 4:1). Fractions with product were evaporated and crystallized from dichloromethane/isopropanol. (Yield: 63 mg, 27%).

¹H NMR (DMSO), δ (ppm): 10.86 (1H, s), 8.23 (1H, s), 8.11 (1H, s), 8.04 (2H, s), 7.80 (1H, d, J=8 Hz), 7.76 (1H, t, J=1.5 Hz), 7.60 (2H, m), 7.47 (4H, m), 7.18 (1H, dd, J=2.0, 8.0 Hz), 4.26 (2H, s), 4.18 (1H, m), 3.40 (2H, d, J=11.5 Hz), 3.09 (2H, m), 3.94 (3H, s), 2.33 (2H, dq, J=2.5, 12.5 Hz), 1.96 (2H, d, J=12.5 Hz).

¹³C NMR (DMSO), δ (ppm): 150.9, 150.7, 139.4, 137.9, 134.9, 131.4, 130, 129.8, 129.5, 128.8, 122.8, 120.7, 118.4, 115.3, 58.8, 52.1, 50.3, 31.6, 24.8.

### Example 8

### 4-(1-((1-(3,5-dimethoxybenzyl)piperidin-4-yl)(methyl)carbamoyl)-1H-imidazol-4-yl)phenyl sulfamate hydrochloride

### Step1: N-(1-(3,5-dimethoxybenzyl)piperidin-4-yl)-4-(4-hydroxyphenyl)-N-methyl-1H-imidazole-1-carboxamide

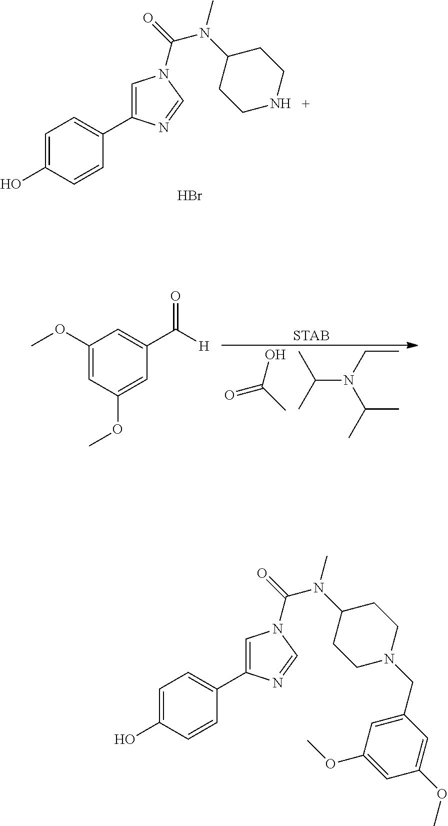

To a suspension of 4-(4-hydroxyphenyl)-N-methyl-N-(piperidin-4-yl)-1H-imidazole-1-carboxamide hydrobromide (Intermediate 12) (1 g, 2.62 mmol) in 1,2-dichloroethane (22.81 mL) was added N,N-diisopropylethylamine (1.81 mL, 10.49 mmol), followed by 3,5-dimethoxybenzaldehyde (0.872 g, 5.25 mmol) and allowed to stir at room temperature for ca 30 min. Then, sodium triacetoxyhydroborate (1.112 g, 5.25 mmol) was added followed by acetic acid (0.150 mL, 2.62 mmol) and the stirring was continued at room temperature for 18 h. Thereupon, the reaction was quenched with ice to leave a solid that was recrystallized from isopropanol. (Yield: 0.628 g, 50%).

### Step2: 4-(1-((1-(3,5-dimethoxybenzyl)piperidin-4-yl)(methyl)carbamoyl)-1H-imidazol-4-yl)phenyl sulfamate

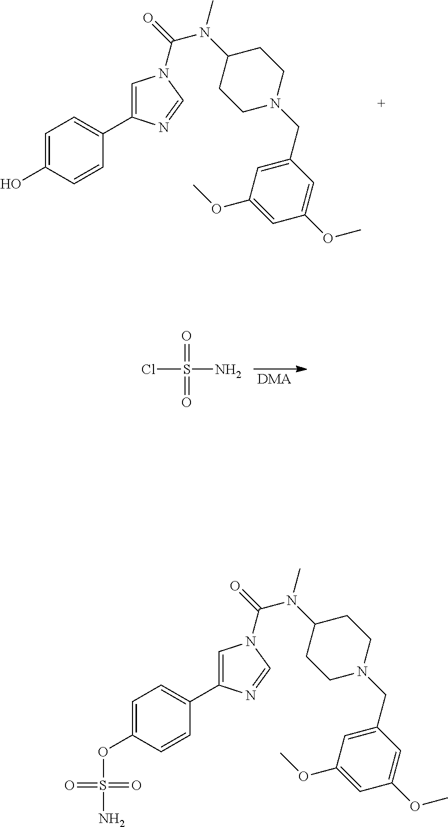

To a turbid solution of N-(1-(3,5-dimethoxybenzyl)piperidin-4-yl)-4-(4-hydroxyphenyl)-N-methyl-1H-imidazole-1-carboxamide (153 mg, 0.340 mmol) in N,N-dimethylacetamide (1.4 mL) was added sulfamoyl chloride (157 mg, 1.358 mmol) and stirred the reaction mixture at room temperature. After 48 h loaded reaction mixture on column chromatography (gradient elution in a mixture of dichloromethane/methanol 49:1, then dichloromethane/methanol/ammonia (25% aq.) 95:5:0.1), followed by one more column chromatography (dichloromethane/methanol 9:1). (Yield: 108 mg, 61%).

### Step3: 4-(1-((1-(3,5-dimethoxybenzyl)piperidin-4-yl)(methyl)carbamoyl)-1H-imidazol-4-yl)phenyl sulfamate hydrochloride

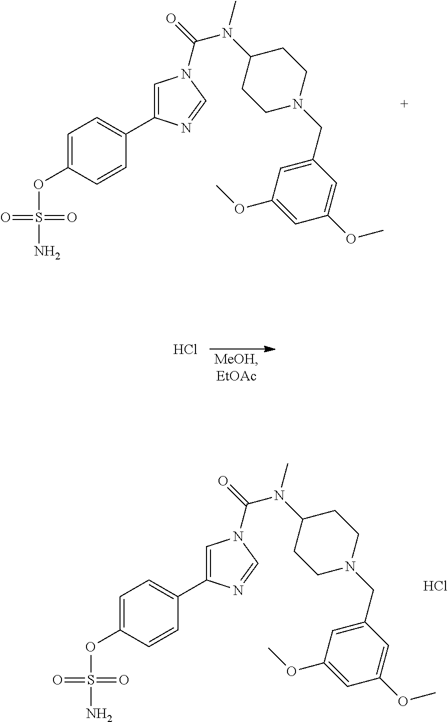

To a chilled suspension of 4-(1-((1-(3,5-dimethoxybenzyl)piperidin-4-yl)(methyl)carbamoyl)-1H-imidazol-4-yl)phenyl sulfamate (104 mg, 0.196 mmol) in ethyl acetate (2 mL)/methanol (2 mL) was added dropwise hydrogen chloride (2 M in ether) (0.393 mL, 0.785 mmol) and allowed to stir at 0° C. for a couple of hours. The solvents were removed under reduced pressure and the residue was triturated from diethyl ether. The precipitate was filtered and dried under vacuum. (Yield: 115 mg, 93%).

¹H NMR (DMSO), δ (ppm): 11.29 (1H, s br), 8.64 (1H, s br), 8.21 (1H, s br), 8.07 (2H, s), 7.96 (2H, d, J=8.7 Hz), 7.34 (2H, d, J=8.7 Hz), 6.87 (2H, d, J=2.1 Hz), 6.55 (1H, t, J=2.1 Hz), 4.20 (1H, br), 4.17 (2H, d, J=5.1 Hz), 3.77 (6H, s), 3.38 (22H, d, J=12.0 Hz), 3.07 (2H, q, J=11.5 Hz), 2.96 (3H, s), 2.44 (2H, dq, J=3.0, 12.5 Hz), 1.95 (2H, d, J=12.0 Hz).

¹³C NMR (DMSO), δ (ppm): 160.6, 150.2, 149.7, 137.8, 137.6, 132, 129.6, 126.4, 122.5, 115.3, 109.1, 101, 58.9, 55.4, 52.3, 50.4, 31.5, 24.6.

### Example 9

### 3-(1-((1-(3,5-dimethoxybenzyl)piperidin-4-yl)(methyl)carbamoyl)-1H-imidazol-4-yl)phenyl sulfamate hydrochloride

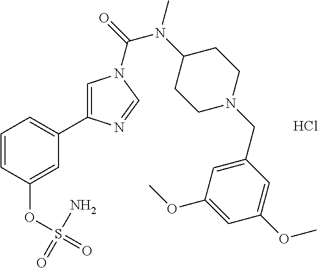 [SMILES: COc1cc(CN2CCC(N(C)C(=O)n3cnc(-c4cccc(OS(N)(=O)=O)c4)c3)CC2)cc(OC)c1 ; Cl]

The title compound was prepared by analogous manner to Example 8 from Intermediate 13.

Appearance: white solid.

¹H NMR (DMSO), δ (ppm): 11.22 (1H, s br), 8.50 (1H, s br), 8.21 (1H, s br), 8.07 (2H, s br), 7.82 (1H, d, J=8.0 Hz), 7.78 (1H, t, J=2.0 Hz), 7.50 (1H, t, J=8.0 Hz), 7.22 (1H, dd, J=2.0, 8.0 Hz), 6.86 (2H, d, J=2.0 Hz), 6.55 (1H, t, J=2.0 Hz), 4.20 (1H, br), 4.17 (2H, d, J=5.1 Hz), 3.77 (6H, s), 3.39 (2H, d, J=11.5 Hz), 3.07 (2H, q, J=11.0 Hz), 2.96 (3H, s), 2.43 (2H, dq, J=2.5, 12.5 Hz), 1.95 (2H, d, J=12.2 Hz).

¹³C NMR (DMSO), δ (ppm): 160.6, 150.7, 150.4, 138.1, 137.9, 133.7, 132, 130.1, 123, 121.1, 118.6, 115.7, 109.1, 101, 58.9, 55.4, 52.2, 50.4, 31.5, 24.7.

### Example 10

### 3-(1-((1-(4-fluoro-3-methoxybenzyl)piperidin-4-yl)(methyl)carbamoyl)-1H-imidazol-4-yl)phenyl sulfamate hydrochloride

### Step1: N-(1-(4-fluoro-3-methoxybenzyl)piperidin-4-yl)-4-(3-hydroxyphenyl)-N-methyl-1H-imidazole-1-carboxamide

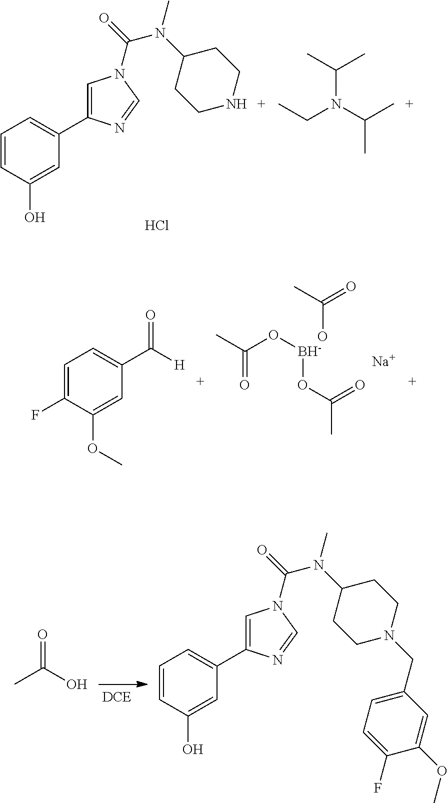

In a 25 mL round-bottomed flask 4-(3-hydroxyphenyl)-N-methyl-N-(piperidin-4-yl)-1H-imidazole-1-carboxamide (250 mg, 0.832 mmol) and 1,2-dichloroethane (7.2 mL) were placed. N,N-diisopropylethylamine (0.58 mL, 3.33 mmol) was added, followed by 4-fluoro-3-methoxybenzaldehyde (257 mg, 1.665 mmol). The mixture was stirred at room temperature for 30 min and then sodium triacetoxyborohydride (353 mg, 1.665 mmol) was added followed by acetic acid (47.6 μL, 0.832 mmol). After 72 h, the reaction mixture was quenched with NaHCO₃ saturated solution. Methanol was added to solution and the precipitate was filtered off. The solvents were removed under reduced pressure and the residue was purified by column chromatography (dichloromethane/methanol 49:1). (Yield: 300 mg, 82%).

### Step2: 3-(1-((1-(4-fluoro-3-methoxybenzyl)piperidin-4-yl)(methyl)carbamoyl)-1H-imidazol-4-yl)phenyl sulfamate

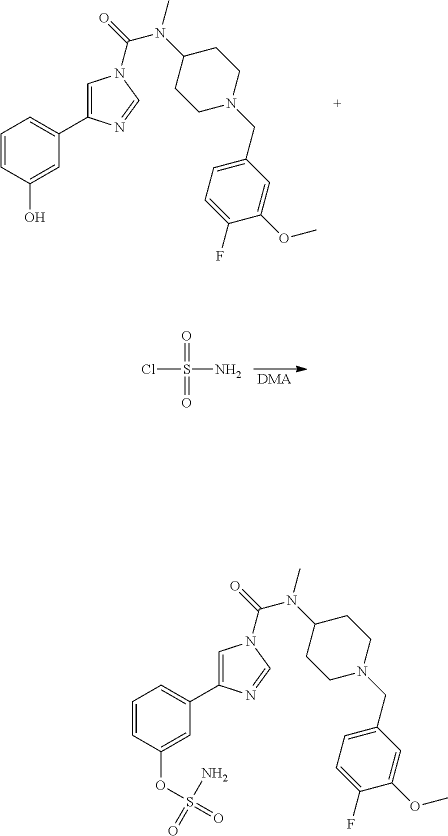

In a 25 mL round-bottomed flask, N-(1-(4-fluoro-3-methoxybenzyl)piperidin-4-yl)-4-(3-hydroxyphenyl)-N-methyl-1H-imidazole-1-carboxamide (200 mg, 0.456 mmol) and N,N-dimethylacetamide (1.8 mL) were placed under inert atmosphere. Sulfamoyl chloride (211 mg, 1.824 mmol) was added and the solution was stirred at room temperature for 18 h. The reaction mixture was quenched with water and pyridine. The solvents were removed under reduced pressure and the residue was purified by column chromatography (dichloromethane/methanol 1:0, 49:1, 9:1). (Yield: 156 mg, 63%).

### Step3: 3-(1-((1-(4-fluoro-3-methoxybenzyl)piperidin-4-yl)(methyl)carbamoyl)-1H-imidazol-4-yl)phenyl sulfamate hydrochloride

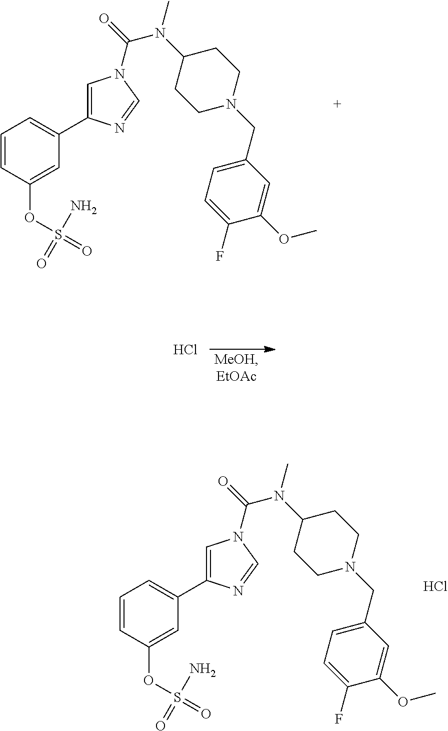

To a chilled suspension of 3-(1-((1-(4-fluoro-3-methoxybenzyl)piperidin-4-yl)(methyl)carbamoyl)-1H-imidazol-4-yl)phenyl sulfamate (156 mg, 0.301 mmol) in a mixture of ethyl acetate (3.5 mL) and methanol (3.5 mL) was added dropwise hydrogen chloride (2 M in ether) (0.603 mL, 1.206 mmol) and allowed to stir at room temperature for a couple of hours. Then, the solvents were removed under reduced pressure and the residue was triturated from diethyl ether. The precipitate was filtered and dried under vacuum. (Yield: 170 mg, 92%).

¹H NMR (DMSO), δ (ppm): 11.37 (1H, s br), 8.56 (1H, s br), 8.23 (1H, s), 8.08 (2H, s), 7.82 (1H, d, J=8.0 Hz), 7.79 (1H, t, J=2.0 Hz), 7.67 (1H, dd, J=1.5, 8.5 Hz), 7.51 (1H, t, J=7.9 Hz), 7.28 (dd, J=8.3, 11.4 Hz), 7.23 (1H, ddd, J=1.0, 2.3, 8.1 Hz), 7.10 (1H, m), 4.21 (1H, m), 4.24 (d, J=4.9 Hz), 3.89 (3H, s), 3.39 (2H, d, J=11.0 Hz), 3.08 (2H, q, J=11.5 Hz), 2.96 (3H, s), 2.43 (2H, dq, J=3.0, 12.5 Hz), 1.95 (2H, d, J=12.5 Hz).

¹³C NMR (DMSO), δ (ppm): 152.8, 151.1, 150.7, 150.4, 147.1, 147.1, 137.9, 137.9, 133.5, 130.2, 126.7, 126.6, 123.9, 123.9, 123.1, 121.2, 118.6, 117, 116, 115.9, 115.7, 58.4, 56.1, 52.2, 50.2, 31.5, 24.7.

### Example 11

### 3-(1-((1-(3-methoxybenzyl)piperidin-4-yl)(methyl)carbamoyl)-1H-imidazol-4-yl)phenyl sulfamate hydrochloride

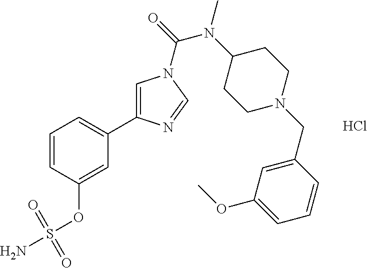 [SMILES: COc1cccc(CN2CCC(N(C)C(=O)n3cnc(-c4cccc(OS(N)(=O)=O)c4)c3)CC2)c1 ; Cl]

The title compound was prepared by analogous manner to Example 10 from 3-methoxy benzaldehyde.

Appearance: white solid.

¹H NMR (DMSO), δ (ppm): 11.28 (1H, s br), 8.28 (1H, s), 8.13 (1H, s), 8.05 (2H, s), 7.80 (1H, d, J=8.0 Hz), 7.77 (1H, t, J=1.7 Hz), 7.48 (1H, t, J=8.0 Hz), 7.35 (2H, m), 7.20 (1H, m), 7.14 (1H, d, J=7.5 Hz), 7.0 (1H, dd, J=2.5, 8.5 Hz), 4.22 (2H, d, J=5.2 Hz), 4.19 (1H, s br), 3.79 (3H, s), 3.38 (2H, m), 3.07 (2H, q, J=11.5 Hz), 2.95 (3H, s), 2.41 (2H, dq, J=3.1, 12.7 Hz), 1.95 (2H, d, J=13.0 Hz).

¹³C NMR (DMSO), δ (ppm): 159.3, 150.8, 150.7, 137.9, 134.7, 131.3, 130, 129.8, 123.3, 122.9, 120.8, 118.4, 116.6, 115.4, 115.1, 68.6, 58.7, 55.2, 52.1, 50.3, 31.4, 24.7.

### Example 12

### 4-(1-((1-(4-fluoro-3-methoxybenzyl)piperidin-4-yl)(methyl)carbamoyl)-1 H-imidazol-4-yl)phenyl sulfamate oxalate

### Step1: N-(1-(4-fluoro-3-methoxybenzyl)piperidin-4-yl)-4-(4-hydroxyphenyl)-N-methyl-1 H-imidazole-1-carboxamide

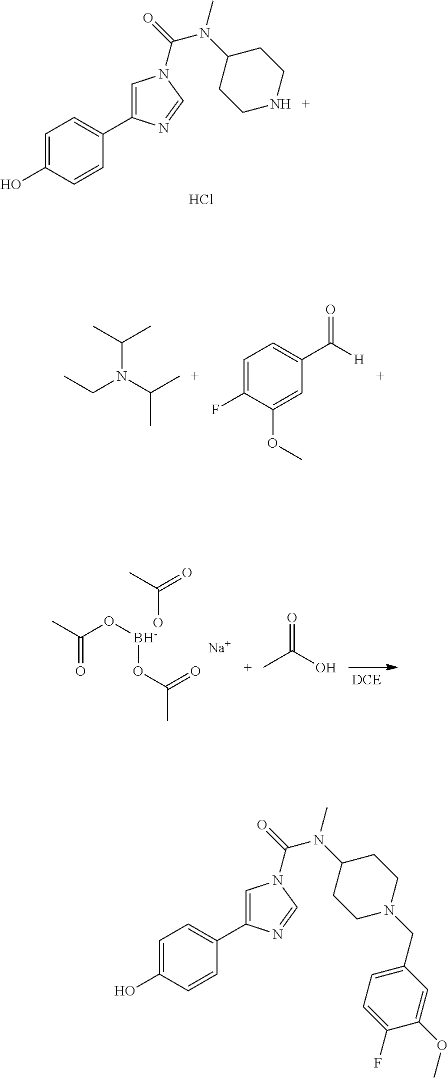

In a 50 mL round-bottomed flask 4-(4-hydroxyphenyl)-N-methyl-N-(piperidin-4-yl)-1H-imidazole-1-carboxamide hydrobromide (Intermediate 12) (500 mg, 1.311 mmol) and 1,2-dichloroethane (11.4 mL) were placed. N,N-diisopropylethylamine (0.916 mL, 5.25 mmol) was added, followed by 4-fluoro-3-methoxybenzaldehyde (404 mg, 2.62 mmol). The mixture was stirred at room temperature for 1 h and sodium triacetoxyborohydride (556 mg, 2.62 mmol) was added followed by acetic acid (0.075 mL, 1.311 mmol). After 48 h, the reaction mixture was quenched with NaHCO₃ saturated solution and extracted into a mixture of dichloromethane/methanol 9:1. The combined organic layers were dried over anhydrous Na₂SO₄, filtered through a short pad of silica/celite and concentrated. The residue was purified by column chromatography (dichloromethane/methanol 9:1) to afford a white solid. (Yield: 140 mg, 15%).

### Step2: 4-(1-((1-(4-fluoro-3-methoxybenzyl)piperidin-4-yl)(methyl)carbamoyl)-1H-imidazol-4-yl)phenyl sulfamate

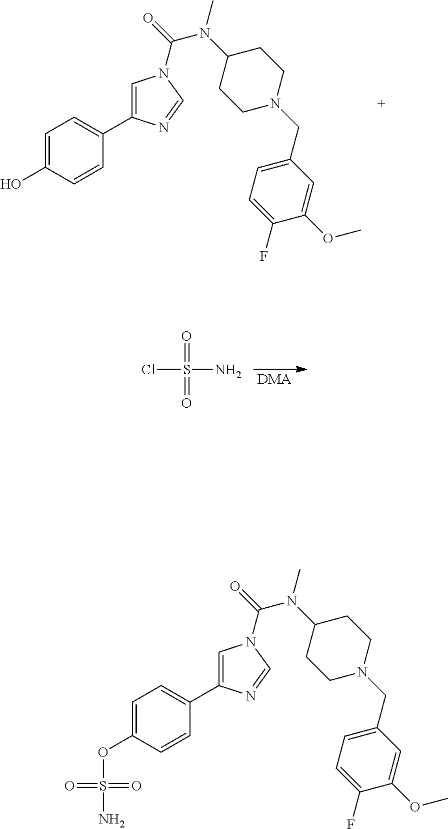

In a 25 mL round-bottomed flask, N-(1-(4-fluoro-3-methoxybenzyl)piperidin-4-yl)-4-(4-hydroxyphenyl)-N-methyl-1H-imidazole-1-carboxamide (138 mg, 0.315 mmol) and N,N-dimethylacetamide (1.3 mL) were placed under inert atmosphere. Sulfamoyl chloride (145 mg, 1.259 mmol) was added and the solution was stirred at room temperature for 48 h. The reaction mixture was quenched with a small amount of water and pyridine and loaded on top of chromatographic column, then eluted with a mixture of dichloromethane/methanol 1:0, 9:1, then dichloromethane/methanol/ammonia (aq. 25%) 9:1:0.1). (Yield: 51.1 mg, 52%).

### Step3: 4-(1-((1-(4-fluoro-3-methoxybenzyl)piperidin-4-yl)(methyl)carbamoyl)-1H-imidazol-4-yl)phenyl sulfamate oxalate

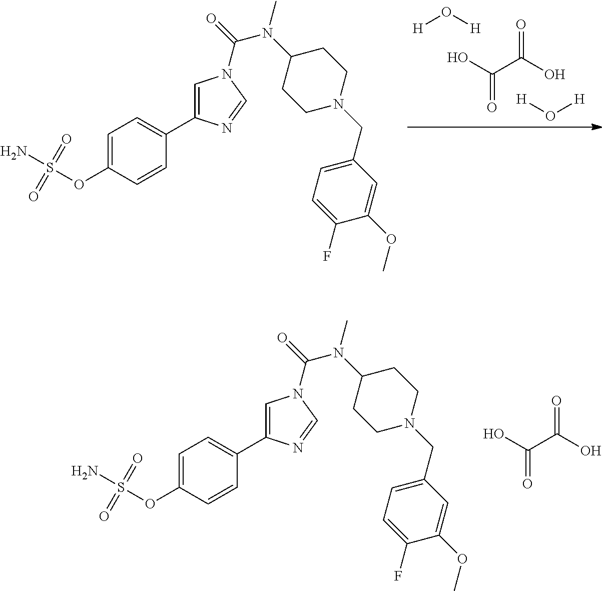

To a chilled suspension of 4-(1-((1-(4-fluoro-3-methoxybenzyl)piperidin-4-yl)(methyl)carbamoyl)-1H-imidazol-4-yl)phenyl sulfamate (51.1 mg, 0.099 mmol) in methanol (2 mL) was added oxalic acid dihydrate (12.45 mg, 0.099 mmol) and allowed to stir at room temperature for 18 h. Then, the solvents were removed under reduced pressure. Recrystallization from isopropanol afforded a white solid (Yield: 40 mg, 60%).

¹H NMR (DMSO), δ (ppm): 8.15 (1H, s br), 8.04 (1H, s br), 8.0 (2H, s br), 7.91 (2H, d, J=8.5 Hz), 7.30 (2H, d, J=8.5 Hz), 7.27-7.21 (2H, m), 6.99 (1H, s br), 4.05 (1H, s br), 3.97 (2H, s br), 3.85 (3H, s), 3.23 (2H, s br), 2.95 (3H, s), 2.73 (2H, s br), 2.07 (2H, s br), 1.90 (2H, s br).

¹³C NMR (DMSO), δ (ppm): 163.4, 152.3, 151, 150.7, 149.1, 147.1, 147, 140.1, 139.8, 137.9, 131.8, 126, 122.8, 122.4, 115.9, 115.8, 115.7, 114.6, 59.2, 56, 53.4, 50.9, 31.6, 26.

### Example 13

### 4-(3-carbamoylphenyl)-N-(1-(3-methoxybenzyl)piperidin-4-yl)-N-methyl-1H-imidazole-1-carboxamide hydrochloride

### Step1: 3-(1H-imidazol-4-yl)benzonitrile

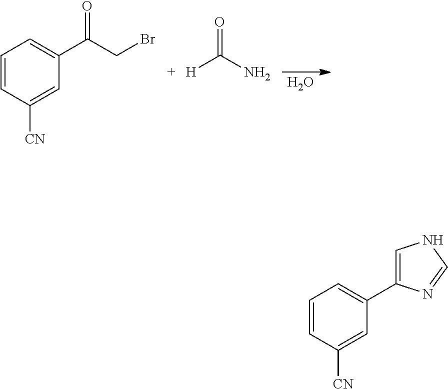

In a 100 mL round-bottomed flask 3-(2-bromoacetyl)benzonitrile (10 g, 44.6 mmol), formamide (21.98 mL, 553 mmol) and water (1.65 mL) were placed. The mixture was heated at 140° C. and stirred for 2 h. Then, it was cooled to room temperature and poured onto 1N HCl solution (100 mL). The resultant precipitate was filtered off and washed with 1N HCl solution. The filtrate's pH was set to 10 by adding 3N NaOH solution. The resultant precipitate was filtered off, washed with water and dried under vacuum. (Yield: 1.95 g, 26%).

### Step2: 3-(1H-imidazol-4-yl)benzamide

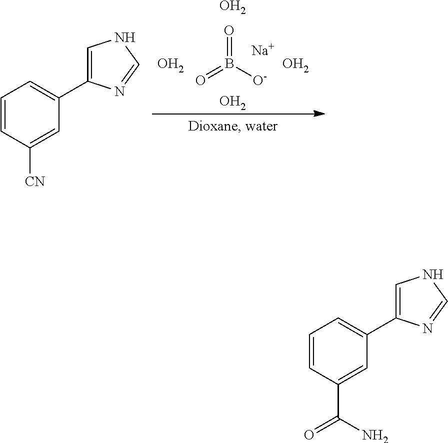

In a 100 mL round-bottomed flask 3-(1H-imidazol-4-yl)benzonitrile (521.6 mg, 3.08 mmol), water (10 mL) and dioxane (10 mL) were placed. Then, sodium perborate tetrahydrate (1309 mg, 8.51 mmol) was added and the mixture was heated at 80° C. and stirred for 48 h. An extra portion of sodium perborate tetrahydrate (750 mg, 4.87 mmol) was added and the mixture was stirred at 80° C. for further 24 h. The reaction mixture was cooled to room temperature, dioxane was removed under reduced pressure. The aqueous phase was extracted several times with a mixture of dichloromethane/isopropanol 7:3. The combined organic layers were dried over MgSO₄ and concentrated. The resultant foam was dissolved in minimal volume of methanol and precipitated with diethyl ether. The precipitate was filtered off and dried under vacuum. (Yield: 380.7 mg, 59%).

### Step3: tert-butyl 4-(4-(3-carbamoylphenyl)-N-methyl-1H-imidazole-1-carboxamido)piperidine-1-carboxylate

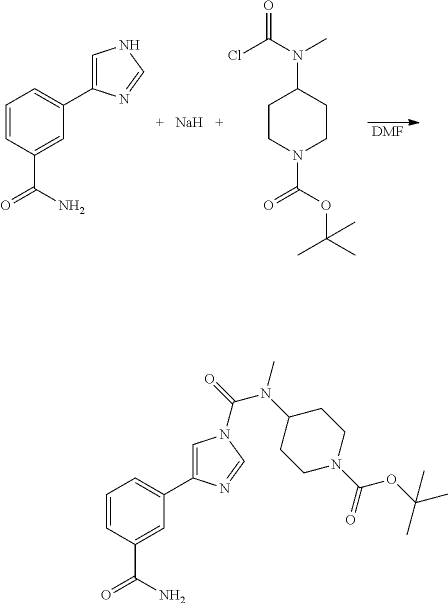

In a 50 mL round-bottomed flask, 3-(1H-imidazol-4-yl)benzamide (0.75 g, 4.01 mmol) and N,N-dimethylformamide (20 mL) were placed under inert atmosphere. The mixture was cooled to 0° C. and sodium hydride (0.192 g, 4.81 mmol) was added portionwise. Then, the reaction mixture was allowed to warm to room temperature and was stirred for 30 minutes. tert-butyl 4-(chlorocarbonyl(methyl)amino)piperidine-1-carboxylate (1.331 g, 4.81 mmol) was added portionwise and the reaction mixture was stirred for 2.5 h. The mixture was cooled to 0° C. and quenched with water. The phases were separated. The aqueous phase was extracted several times with a mixture of dichloromethane/isopropanol 7:3. The combined organic layers were dried over MgSO₄ and concentrated. The resultant residue was purified by column chromatography (dichloromethane/methanol 1:0, 9:1) to yield a colourless oil that was triturated with ethyl ether. The precipitate was filtered and dried under vacuum. (Yield: 0.656 g, 38%).

### Step4: 4-(3-carbamoylphenyl)-N-methyl-N-(piperidin-4-yl)-1H-imidazole-1-carboxamide hydrochloride

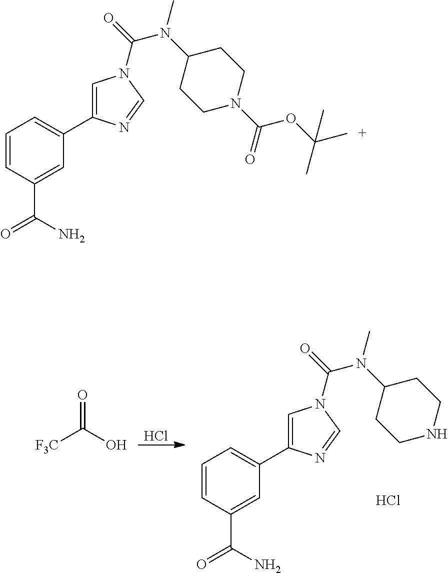

tert-butyl 4-(4-(3-carbamoylphenyl)-N-methyl-1H-imidazole-1-carboxamido)piperidine-1-carboxylate (500 mg, 1.170 mmol) was dissolved in trifluoroacetic acid (2 mL), at 0° C., and the reaction was allowed to stir vigorously at room temperature for 1 h. Trifluoroacetic acid was then removed under reduced pressure and the obtained residue was dissolved in 5 mL of ethyl acetate. The solution was cooled to 0° C. and treated with 2N hydrogen chloride in diethyl ether (0.585 mL, 1.17 mmol). The resultant precipitate was filtered, washed with diethyl ether and dried under vacuum. (Yield: 380 mg, 89%).

### Step5: 4-(3-carbamoylphenyl)-N-(1-(3-methoxybenzyl)piperidin-4-yl)-N-methyl-1H-imidazole-1-carboxamide hydrochloride

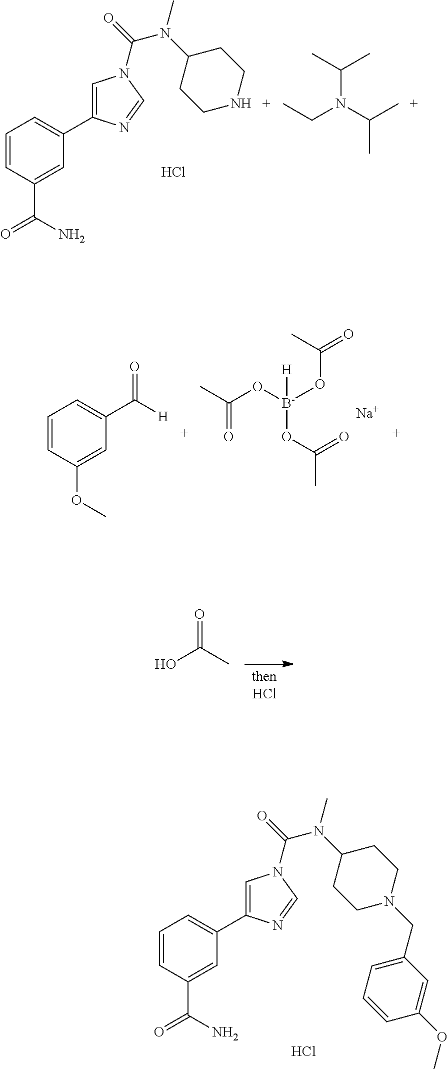

In a 100 mL round-bottomed flask 4-(3-carbamoylphenyl)-N-methyl-N-(piperidin-4-yl)-1H-imidazole-1-carboxamide hydrochloride (0.38 g, 1.044 mmol) and 1,2-dichloroethane (15 mL) were placed. N,N-diisopropylethylamine (0.73 mL, 4.18 mmol) was added, followed by 3-methoxybenzaldehyde (0.254 mL, 2.089 mmol). The mixture was stirred at room temperature for 30 min and then sodium triacetoxyborohydride (0.443 g, 2.089 mmol) was added followed by acetic acid (0.06 mL, 1.044 mmol). The reaction mixture was stirred, at room temperature, overnight. Then, the reaction mixture was quenched with water. The phases were separated and the aqueous phase was extracted several times with a mixture of dichloromethane/isopropanol 7:3. The combined organic layers were dried over MgSO₄, filtered and evaporated. The resultant residue was purified by column chromatography (dichloromethane/methanol 1:0, 9:1) to yield a colourless oil that was triturated with ethyl ether. The precipitate was filtered, washed with diethyl ether and dried under vacuum. The solid was dissolved in ethyl acetate, cooled to 0° C. and treated with 2N hydrogen chloride in diethyl ether (0.585 mL, 1.17 mmol). The reaction mixture was allowed to warm to room temperature and was stirred for 3 h. The precipitate was filtered, washed with diethyl ether and dried under vacuum. (Yield: 0.156 g, 31%, white powder).

¹H NMR (DMSO), δ (ppm): 10.82 (1H, s br), 8.35 (1H, s br), 8.17 (1H, s br), 8.08 (1H, s br), 8.03 (1H, s br), 7.98 (1H, d, J=7.8 Hz), 7.76 (1H, d, J=7.7 Hz), 7.47 (1H, t, J=7.7 Hz), 7.40 (1H, s br), 7.37 (1 h, t, J=8.0 Hz), 7.29 (1H, s br), 7.12 (1H, d, J=7.3 Hz), 7.02 (1H, d, J=8.0 Hz), 4.22 (2H, m), 4.20 (1H, s br), 3.80 (3H, s), 3.10 (2H, q, J=11.0 Hz), 2.96 (3H, s), 2.34 (2H, q, J=12.0 Hz), 1.96 (2H, d, J=12.5 Hz).

¹³C NMR (DMSO), δ (ppm): 167.8, 159.4, 151.1, 140.2, 137.9, 134.7, 133.3, 131.2, 129.9, 128.6, 127.3, 126.1, 123.9, 123.3, 116.7, 115.1, 114.9, 58.8, 55.2, 52.1, 50.5, 31.7, 24.9.

### Example 14

### 4-(1-((1-(3-methoxybenzyl)piperidin-4-yl)(methyl)carbamoyl)-1H-imidazol-4-yl)phenyl sulfamate hydrochloride

### Step1: 1-(4-(benzyloxy)phenyl)ethanone

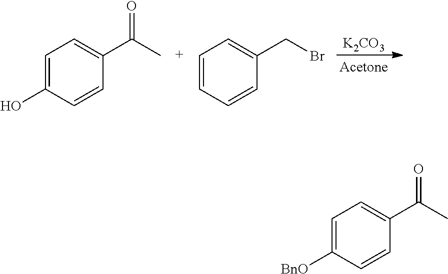

To a clear solution of 1-(4-hydroxyphenyl)ethanone (10 g, 73.4 mmol) in acetone (150 mL) was added potassium carbonate (13.20 g, 95 mmol), followed by benzyl bromide (11.42 mL, 95 mmol), dropwise. The mixture was heated at reflux for 18 h. Reaction mixture was cooled to room temperature, filtered, the filter cake was washed with acetone and the filtrate was evaporated. The resulting white solid was suspended in petroleum ether, filtered and dried to afford a white solid. (Yield: 16.22 g, 93%).

### Step2: 1-(4-(benzyloxy)phenyl)-2-bromoethanone

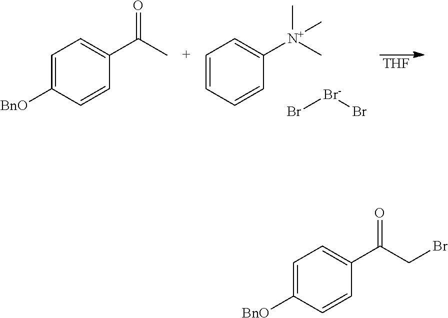

To a clear solution of 1-(4-(benzyloxy)phenyl)ethanone (16.1 g, 71.2 mmol) in tetrahydrofuran (200 mL) was added dropwise a solution of phenyltrimethylammonium tribromide (29.4 g, 78 mmol) in tetrahydrofuran (150 mL) at 0° C. Once the reaction is complete, the insoluble material was filtered off and washed with tetrahydrofuran. The filtrate was evaporated to leave a yellow oil, which was crystallized from isopropanol. (Yield: 17.99 g, 83%).

### Step3: 4-(4-(benzyloxy)phenyl)-1H-imidazole

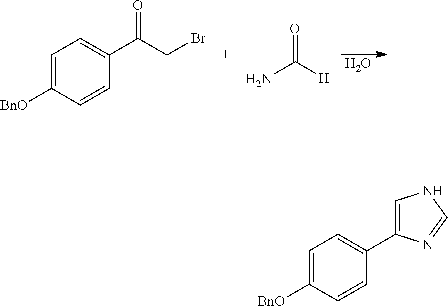

A stirred mixture of 1-(4-(benzyloxy)phenyl)-2-bromoethanone (17.99 g, 59.0 mmol), formamide (29.1 mL, 731 mmol) and water (2 mL) was heated at 140° C. for 7 hours.

Then, poured the mixture onto 100 mL ice-water and the solid was filtered off. The filtrate was basified until pH 12, transferred into a separatory funnel and extracted with a mixture of dichloromethane/isopropanol 7:3. The organic layer was further washed with water, concentrated to afford a brown solid. (Yield: 8.36 g, 57%).

### Step4: tert-butyl 4-(4-(4-(benzyloxy)phenyl)-N-methyl-1H-imidazole-1-carboxamido)piperidine-1-carboxylate

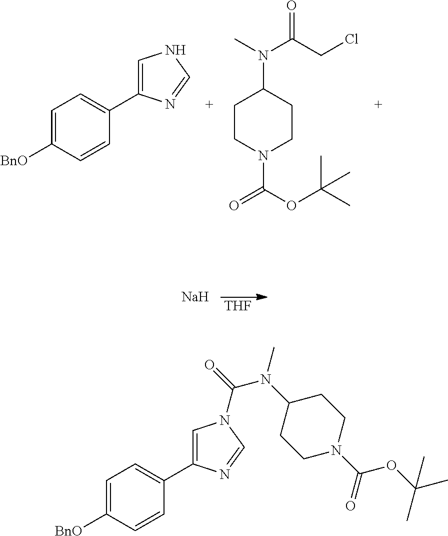

To a slightly turbid solution of 4-(4-(benzyloxy)phenyl)-1H-imidazole (1.78 g, 7.11 mmol) in tetrahydrofuran (28.4 mL) was added sodium hydride (60% in mineral oil dispersion, 0.370 g, 9.25 mmol). After stirring for 15 min, tert-butyl 4-(chlorocarbonyl(methyl)amino)piperidine-1-carboxylate (2.362 g, 8.53 mmol) was added and the reaction mixture was stirred at room temperature for 1 h. The obtained precipitate was filtered off, washed with diethyl ether and dried under vacuum. (Yield: 3.84 g, quantitative).

### Step5: 4-(4-(benzyloxy)phenyl)-N-methyl-N-(piperidin-4-yl)-1H-imidazole-1-carboxamide hydrochloride

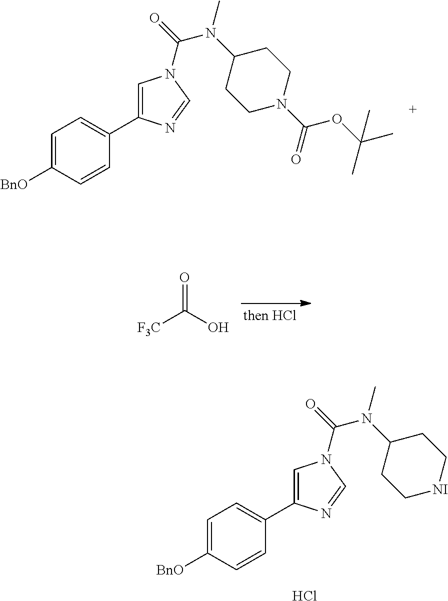

tert-butyl 4-(4-(4-(benzyloxy)phenyl)-N-methyl-1H-imidazole-1-carboxamido)piperidine-1-carboxylate (3.49 g, 7.11 mmol) was dissolved in trifluoroacetic acid (21.92 mL, 285 mmol), at 0° C., and stirred for 30 min. Then, trifluoroacetic acid was removed under reduced pressure and the residue was dissolved in methanol (14.23 mL), chilled in ice/water bath, and treated with hydrogen chloride (2 M in diethyl ether) (7.11 mL, 14.23 mmol). Continued stirring at room temperature until a thick white suspension was formed. The solvents were removed under reduced pressure and the white residue was recrystallized from isopropanol. (Yield: 2.9 g, 86%).

### Step6: 4-(4-(benzyloxy)phenyl)-N-(1-(3-methoxybenzyl)piperidin-4-yl)-N-methyl-1H-imidazole-1-carboxamide

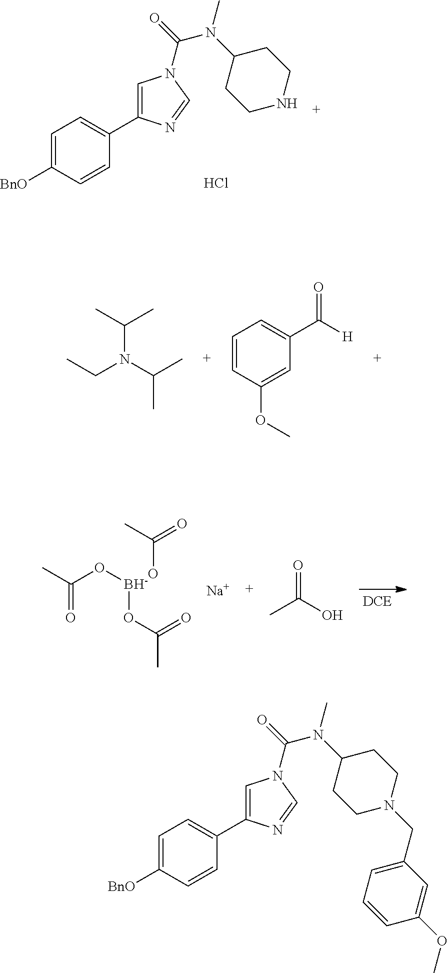

To a suspension of 4-(4-(benzyloxy)phenyl)-N-methyl-N-(piperidin-4-yl)-1H-imidazole-1-carboxamide hydrochloride (1.5 g, 3.51 mmol) in 1,2-dichloroethane (30.6 mL) was added N,N-diisopropylethylamine (2.454 mL, 14.05 mmol), followed by 3-methoxybenzaldehyde (0.855 mL, 7.03 mmol). After stirring for 30 min, at room temperature, sodium triacetoxyhydroborate (1.489 g, 7.03 mmol) was added, followed by acetic acid (0.201 mL, 3.51 mmol). The reaction was allowed to stir at room temperature for 18 h. Once reaction is complete, quenched upon addition of crushed ice, transferred mixture into a separatory funnel and partitioned between water and a mixture of dichloromethane/isopropanol 7:3. The two phases were separated and the aqueous phase was further extracted with dichloromethane/isopropanol 7:3. The combined organic layers were dried over anhydrous Na₂SO₄, filtered through a short pad of silica/celite and concentrated. The residue was recrystallized from isopropanol. (Yield: 1.03 g, 49%).

### Step7: 4-(4-hydroxyphenyl)-N-(1-(3-methoxybenzyl)piperidin-4-yl)-N-methyl-1H-imidazole-1-carboxamide

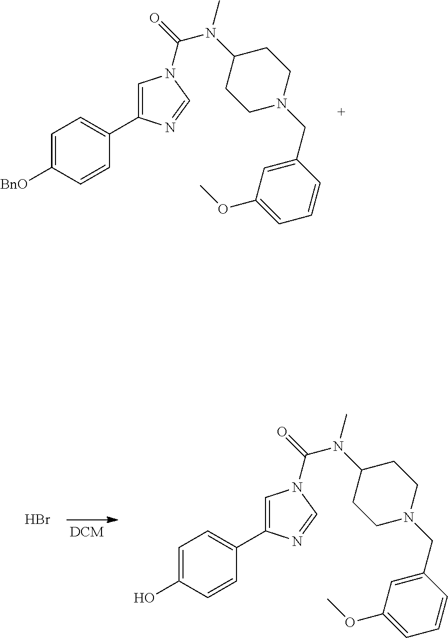 [SMILES: COc1cccc(CN2CCC(N(C)C(=O)n3cnc(-c4ccc(OCc5ccccc5)cc4)c3)CC2)c1 ; Br ; COc1cccc(CN2CCC(N(C)C(=O)n3cnc(-c4ccc(O)cc4)c3)CC2)c1]

To a cold suspension of 4-(4-(benzyloxy)phenyl)-N-(1-(3-methoxybenzyl)piperidin-4-yl)-N-methyl-1H-imidazole-1-carboxamide (780 mg, 1.528 mmol) in dichloromethane (12.2 mL) was added dropwise HBr (33% in AcOH) (0.251 mL, 1.528 mmol) and allowed to stir at room temperature for 3 h. Quenched reaction with water and neutralized upon careful addition of a saturated aqueous solution of Na₂CO₃. Then, the mixture was transferred into a separatory funnel and extracted with a mixture of dichloromethane/methanol 9:1 until no further material could be extracted. The combined organic layers were dried over anhydrous Na₂SO₄, filtered, concentrated and the residue purified by column chromatography using gradient elution (dichloromethane/methanol). (Yield: 556.6 mg, 82%).

### Step8: 4-(1-((1-(3-methoxybenzyl)piperidin-4-yl)(methyl)carbamoyl)-1H-imidazol-4-yl)phenyl sulfamate

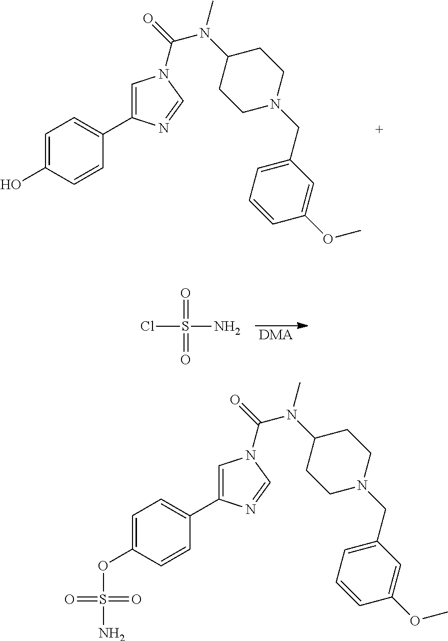

To a clear solution of 4-(4-hydroxyphenyl)-N-(1-(3-methoxybenzyl)piperidin-4-yl)-N-methyl-1H-imidazole-1-carboxamide (250 mg, 0.595 mmol) in N,N-dimethylacetamide (2.4 mL) was added sulfamoyl chloride (275 mg, 2.378 mmol). After completion of the reaction, it was quenched by addition of a mixture of water and pyridine. The solvents were removed under reduced pressure and the residue was purified by column chromatography (dichloromethane/methanol 1:0, 49:1, 9:1, then dichloromethane/methanol/ammonia (aq. 25%) 7:1:0.2). (Yield: 232 mg, 74%).

### Step9: 4-(1-((1-(3-methoxybenzyl)piperidin-4-yl)(methyl)carbamoyl)-1H-imidazol-4-yl)phenyl sulfamate hydrochloride

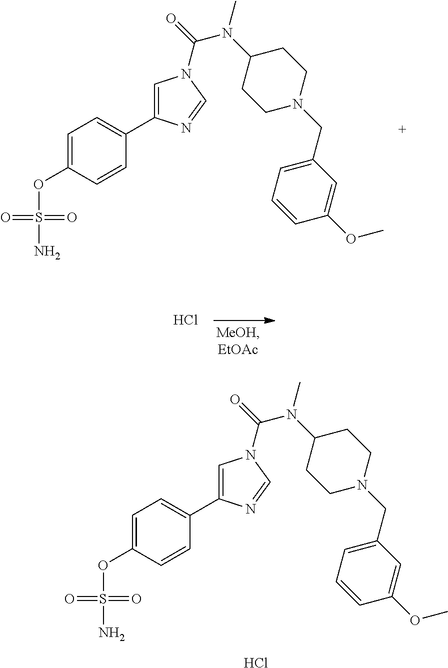 [SMILES: COc1cccc(CN2CCC(N(C)C(=O)n3cnc(-c4ccc(OS(N)(=O)=O)cc4)c3)CC2)c1 ; Cl ; COc1cccc(CN2CCC(N(C)C(=O)n3cnc(-c4ccc(OS(N)(=O)=O)cc4)c3)CC2)c1 ; Cl]

To a suspension of 4-(1-((1-(3-methoxybenzyl)piperidin-4-yl)(methyl)carbamoyl)-1H-imidazol-4-yl)phenyl sulfamate (191 mg, 0.382 mmol) in a mixture of ethyl acetate (5 mL) and methanol (5 mL) was added HCl (2 M in ether) (1.434 mL, 2.87 mmol) at 0° C. The reaction mixture was allowed to stir in the cold for 4 h. The resultant precipitate was filtered off and dried under vacuum to give an off white solid. (Yield: 225 mg, 99%).

¹H NMR (DMSO), d (ppm): 11.31 (1H, s br), 8.68 (1H, s), 8.22 (1H, s), 8.07 (2H, s), 7.96 (2H, d, J=8.3 Hz), 7.35 (4H, m), 7.14 (1H, d, J=7.3 Hz), 7.0 (1H, dd, J=1.5, 8.0 Hz), 4.23 (2H, d, J=4.8 Hz), 4.20 (1H, br), 3.80 (3H, s), 3.38 (2H, m), 3.08 (2H, q, J=11 Hz), 2.96 (3H, s), 2.43 (2H, mq, J=13.0 Hz), 1.95 (2H, d, J=12.5 Hz).

¹³C NMR (DMSO), δ (ppm): 159.4, 150.1, 149.7, 137.8, 137.4, 131.3, 129.9, 129.4, 126.5, 123.3, 122.5, 116.6, 115.4, 115.2, 58.7, 55.2, 52.2, 50.3, 31.6, 24.6.

### Example 15

### 3-(1-((1-(4-hydroxyphenyl)piperidin-4-yl)(methyl)carbamoyl)-1H-imidazol-4-yl)phenyl sulfamate hydrochloride

### Step1: 3-(1H-imidazol-4-yl)phenol

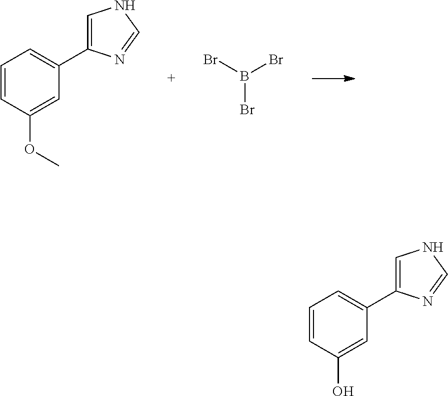

In a 100 mL round-bottomed flask, 4-(3-methoxyphenyl)-1H-imidazole (Intermediate 2) (2.1358 g, 12.26 mmol) and anhydrous dichloromethane (100 mL) were placed under inert atmosphere. The mixture was cooled to −78° C. and boron tribromide (3.49 ml, 36.8 mmol) was added dropwise. The reaction was stirred at −78° C. for 30 min, then was allowed to warm up to room temperature and stirred overnight. The reaction mixture was cooled to 0° C., quenched with water and stirred for 1 h. Dichloromethane was removed under vacuum and the aqueous solution was neutralized with NaHCO₃ saturated solution. The resultant precipitate was filtered, washed with water, diethyl ether and dried under vacuum. (Yield: 1.105 g, 56%).

### Step2: 4-(3-hydroxyphenyl)-N-(1-(4-methoxyphenyl)piperidin-4-yl)-N-methyl-1H-imidazole-1-carboxamide

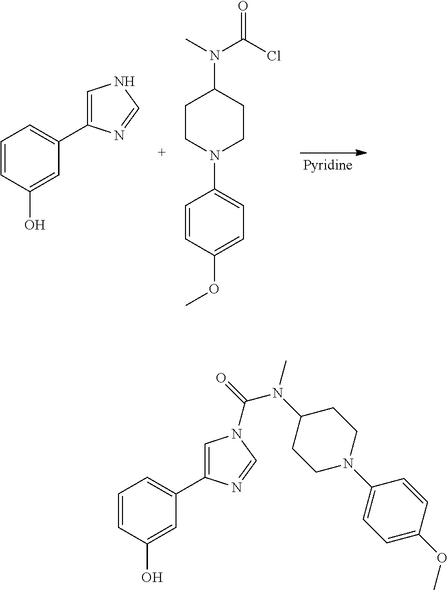 [SMILES: Oc1cccc(-c2c[nH]cn2)c1 ; COc1ccc(N2CCC(N(C)C(=O)Cl)CC2)cc1 ; COc1ccc(N2CCC(N(C)C(=O)n3cnc(-c4cccc(O)c4)c3)CC2)cc1]

A mixture of 3-(1H-imidazol-4-yl)phenol (500 mg, 3.12 mmol) and 1-(4-methoxyphenyl)piperidin-4-yl(methyl)carbamic chloride (intermediate 6) (1147 mg, 4.06 mmol) in Pyridine (25 ml) was heated at 90° C. for 3 h. Then, the reaction was allowed to cool to room temperature and transferred to a separatory funnel, diluted with 100 mL of dichloromethane and washed with 1N HCl aqueous solution and water. The organic phase was dried over MgSO₄, concentrated and the residue was triturated with ethyl ether. The precipitate was filtered and dried under vacuum. (Yield: 417 mg, 33%).

### Step3: 3-(1-((1-(4-methoxyphenyl)piperidin-4-yl)(methyl)carbamoyl)-1H-imidazol-4-yl)phenyl sulfamate

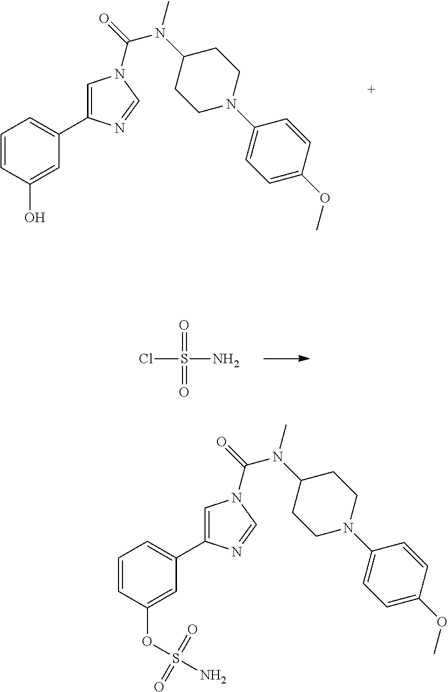 [SMILES: COc1ccc(N2CCC(N(C)C(=O)n3cnc(-c4cccc(O)c4)c3)CC2)cc1 ; NS(=O)(=O)Cl ; COc1ccc(N2CCC(N(C)C(=O)n3cnc(-c4cccc(OS(N)(=O)=O)c4)c3)CC2)cc1]

In a 25 mL round-bottomed flask, 4-(3-hydroxyphenyl)-N-methyl-N-(1-phenylpiperidin-4-yl)-1H-imidazole-1-carboxamide hydrobromide (200 mg, 0.437 mmol) and N,N-dimethylacetamide (5 mL) were placed under inert atmosphere. Sulfamoyl chloride (455 mg, 3.94 mmol) was added and the solution was stirred at room temperature for 4 h. The reaction mixture was quenched with water and neutralized with saturated NaHCO₃ aqueous solution. The resultant precipitate was filtered off, washed with water, diethyl ether and dried under vacuum. (Yield: 328 mg, 69%).

### Step4: 3-(1-((1-(4-hydroxyphenyl)piperidin-4-yl)(methyl)carbamoyl)-1H-imidazol-4-yl)phenyl sulfamate hydrobromide

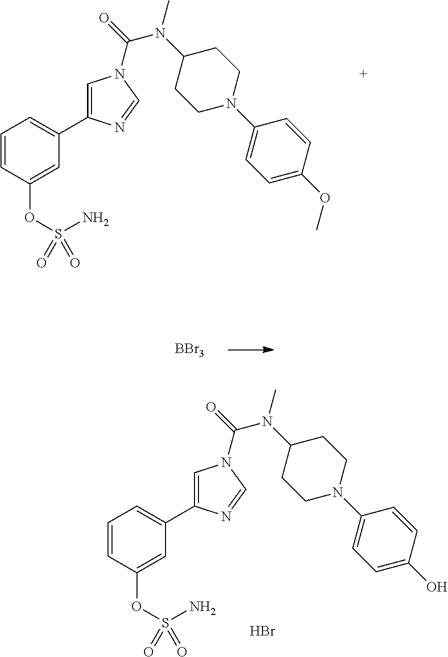 [SMILES: COc1ccc(N2CCC(N(C)C(=O)n3cnc(-c4cccc(OS(N)(=O)=O)c4)c3)CC2)cc1 ; "BBr3" ; CN(C(=O)n1cnc(-c2cccc(OS(N)(=O)=O)c2)c1)C1CCN(c2ccc(O)cc2)CC1 ; Br]

In a 25 mL pear flask, 3-(1-((1-(4-methoxyphenyl)piperidin-4-yl)(methyl)carbamoyl)-1H-imidazol-4-yl)phenyl sulfamate (120 mg, 0.247 mmol) and anhydrous dichloromethane (2 mL) were placed under inert atmosphere. The mixture was cooled to −78° C. and boron tribromide (0.07 mL, 0.741 mmol) was added dropwise. The reaction was stirred at −78° C. for 20 min, then was allowed to warm to room temperature and stirred for 5 h. The reaction mixture was cooled to 0° C., quenched with water and stirred for 30 min. The precipitate was filtered, washed with water and diethyl ether and dried under vacuum. (Yield: 72 mg, 62%, white solid).

¹H NMR (DMSO), δ (ppm): 12.72 (1H, s br), 10.10 (1H, s br), 8.66 (1H, s br), 8.29 (1H, s br), 8.10 (2H, s br), 7.84 (1H, ddd, J=1.0, 1.5, 7.5 Hz), 7.80 (1H, t, J=1.9 Hz), 7.71 (2H, d, J=8.9 Hz), 7.53 (1H, t, J=8.0 Hz), 7.24 (1H, ddd, J=0.8, 2.3, 8.1 Hz), 6.91 (2H, d, J=8.9 Hz), 4.47 (1H, m), 3.80 (1H, m), 3.55 (2H, d, J=11.0 Hz), 3.03 (3H, s), 2.68 (2H, q, J=13.5 Hz), 2.05 (2H, d, J=12.0 Hz).

¹³C NMR (DMSO), δ (ppm): 158.3, 150.7, 150.4, 138, 137.6, 133.8, 133.2, 130.3, 123.1, 122.7, 121.4, 118.7, 116.2, 116, 54.8, 51.4, 31.6, 25.3.

### Example 16

### N-(1-benzylpiperidin-4-yl)-N-methyl-4-(3-ureidophenyl)-1H-imidazole-1-carboxamide hydrochloride

### Step1: N-(1-benzylpiperidin-4-yl)-N-methyl-4-(3-ureidophenyl)-1H-imidazole-1-carboxamide

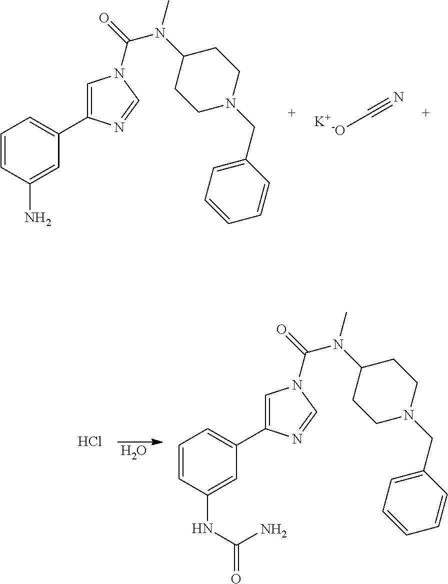

In a 25 mL round-bottomed flask, 4-(3-aminophenyl)-N-(1-benzylpiperidin-4-yl)-N-methyl-1H-imidazole-1-carboxamide (600 mg, 1.540 mmol) (Intermediate 7) and water (1 mL) were placed to give a white suspension. Hydrogen chloride (0.770 mL, 1.540 mmol) was added and the suspension was cooled to 0° C. Potassium cyanate (150 mg, 1.849 mmol) was added in portions and the reaction mixture was allowed to warm up to room temperature and stirred for 8 h. Another portion of hydrogen chloride and potassium cyanate were added. After 6 h, the mixture was filtered and washed with water. The solid was purified by chromatography (dichloromethane/methanol 49:1, 19:1, 9:1). Product was triturated from hot ethyl acetate and diisopropylether. (Yield: 425 mg, 54%).

### Step2: N-(1-benzylpiperidin-4-yl)-N-methyl-4-(3-ureidophenyl)-1H-imidazole-1-carboxamide hydrochloride

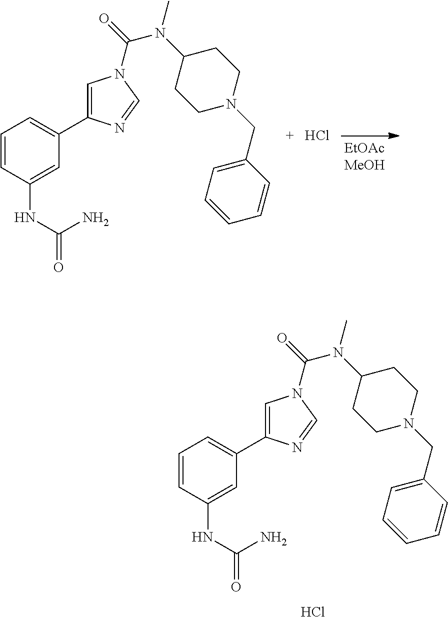 [SMILES: CN(C(=O)n1cnc(-c2cccc(NC(N)=O)c2)c1)C1CCN(Cc2ccccc2)CC1 ; Cl ; CN(C(=O)n1cnc(-c2cccc(NC(N)=O)c2)c1)C1CCN(Cc2ccccc2)CC1 ; Cl]

To a suspension of N-(1-benzylpiperidin-4-yl)-N-methyl-4-(3-ureidophenyl)-1H-imidazole-1-carboxamide (425 mg, 0.983 mmol) in ethyl acetate (5 mL)/methanol (5 mL) added HCl (2 M in ether) (3.68 mL, 7.37 mmol) at 0° C. The reaction mixture was allowed to stir in the cold for 10 min, then for 2 h at room temperature. The precipitate was filtered, washed with ether. Recrystallization from a mixture of dichloromethane/isopropanol afforded white solid (300 mg, 55%).

¹H NMR (DMSO), δ (ppm): 11.33 (1H, s br), 8.99 (1H, s br), 8.97 (1H, s br), 8.16 (1H, s br), 7.91 (1H, t, J=1.7 Hz), 7.64 (2H, m), 7.45 (3H, m), 7.38 (2H, m), 7.30 (1H, t, J=8.1 Hz), 5.6 (2H, br), 4.25 (2H, d, J=5.2 Hz), 4.20 (1H, s br), 3.39 (2H, d, J=11.3 Hz), 3.09 (2H, q, J=10.5 Hz), 2.96 (3H, s), 2.42 (2H, dq, J=3.3, 12.8 Hz), 1.96 (2H, d, J=12.1 Hz).

¹³C NMR (DMSO), δ (ppm): 156.1, 149.6, 141.2, 137.4, 136.9, 131.4, 131.4, 129.8, 129.4, 129.2, 128.8, 118.4, 118, 115.3, 114.6, 58.7, 52.3, 50.2, 31.5, 24.6.

### Example 17

### N-(1-(3-methoxybenzyl)piperidin-4-yl)-N-methyl-4-(3-ureidophenyl)-1 H-imidazole-1-carboxamide hydrochloride

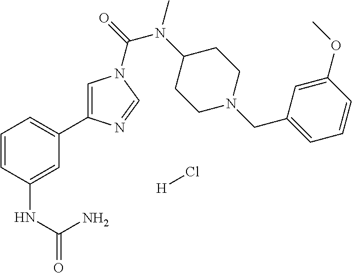 [SMILES: COc1cccc(CN2CCC(N(C)C(=O)n3cnc(-c4cccc(NC(N)=O)c4)c3)CC2)c1 ; Cl]

The title compound was prepared by analogous manner to Example 16 from Intermediate 8.

Appearance: light beige solid.

¹H NMR (DMSO), δ (ppm): 10.43 (1H, s br), 8.63/1H, s br), 8.12 (1H, s br), 7.88 (1H, s br), 7.38 (1H, t, J=7.9 Hz), 7.35 (1H, d, J=7.7 Hz), 7.31 (1H, d, J=8.1 Hz), 7.23 (1H, m), 7.22 (1H, t, J=7.9 Hz), 7.10 (1H, d, J=7.4 Hz), 7.03 (1H, d, J=8.2 Hz), 5.86 (2H, s), 4.23 (2H, d, J=4.3 Hz), 4.16 (1H, s br), 3.80 (3H, s), 3.41 (2H, m), 3.10 (2H, m), 2.93 (3H, s), 2.27 (2H, q, J=12.5 Hz), 1.97 (2H, d, J=12.5 Hz).

¹³C NMR (DMSO), δ (ppm): 159.4, 156, 151.1, 140.9, 140.9, 137.6, 133.6, 131.2, 130, 128.8, 123.2, 117.9, 116.7, 116.7, 115.1, 114.3, 114.2, 58.9, 55.2, 52.1, 50.5, 31.6, 25.

### Example 18

### N-(1-(benzo[d][1,3]dioxol-5-ylmethyl)piperidin-4-yl)-N-methyl-4-(3-ureidophenyl)-1H-imidazole-1-carboxamide hydrochloride

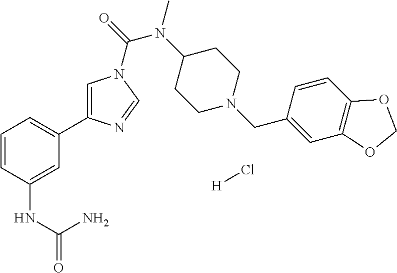

The title compound was prepared by analogous manner to Example 16 from Intermediate 14.

Appearance: off-white solid.

¹H NMR (DMSO), δ (ppm): 10.07 (1H, s br), 8.88 (1H, s br), 8.79 (1H, s br), 8.10 (1H, s br), 7.90 (1H, s br), 7.37 (2H, m), 7.28 (2H, m), 7.03 (1H, d, J=8.2 Hz), 6.98 (1H, d, J=7.9 Hz), 6.07 (2H, s), 4.19 (1H, br), 4.16 (2H, d, J=4.8 Hz), 3.38 (2H, d, J=11.3 Hz, 3.04 (2H, q, J=11.0 Hz), 2.95 (3H, s), 2.37 (2H, mq, J=13.5 Hz), 1.96 (2H, d, J=12.5 Hz).

¹³C NMR (DMSO), δ (ppm): 156.1, 149.9, 148.1, 147.4, 141.2, 137.7, 137.5, 130.6, 129.1, 125.5, 123.2, 118.3, 117.7, 115.1, 114.6, 111.3, 108.4, 101.5, 58.5, 52.3, 50, 31.6, 24.7.

### Example 19

### 4-(1-((1-(3,5-dimethoxybenzyl)piperidin-4-yl)(methyl)carbamoyl)-1H-imidazol-4-yl)phenyl sulfamate maleate

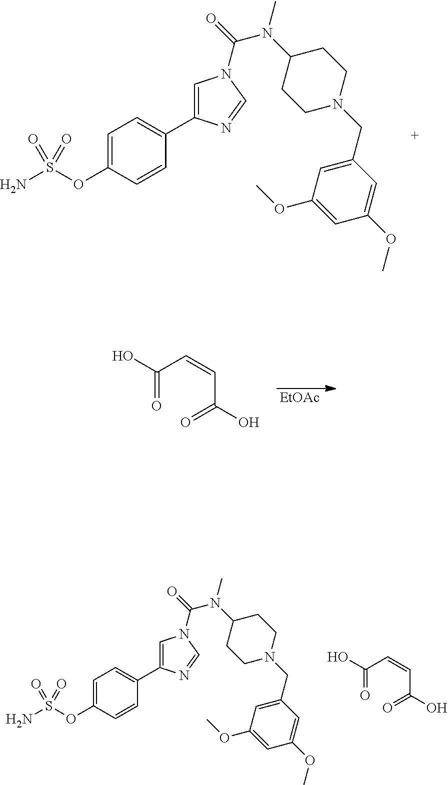 [SMILES: "EtOAc" ; COc1cc(CN2CCC(N(C)C(=O)n3cnc(-c4ccc(OS(N)(=O)=O)cc4)c3)CC2)cc(OC)c1 ; O=C(O)/C=C\C(=O)O ; COc1cc(CN2CCC(N(C)C(=O)n3cnc(-c4ccc(OS(N)(=O)=O)cc4)c3)CC2)cc(OC)c1 ; O=C(O)/C=C\C(=O)O]

4-(1-((1-(3,5-dimethoxybenzyl)piperidin-4-yl)(methyl)carbamoyl)-1H-imidazol-4-yl)phenyl sulfamate (200 mg, 0.378 mmol) (Example 8, step 2) and maleic acid (43.8 mg, 0.378 mmol) were refluxed in Ethyl acetate (5 ml) for 30 min. The reaction mixture was cooled to room temperature and the resultant precipitate was filtered, washed with ethyl ether and dried. (Yield: 228 mg, 94%, white solid).

¹H NMR (DMSO), δ (ppm): 9.29 (1H, br), 8.16 (1H, s br), 8.05 (1H, s br), 8.03 (2H, s br), 7.91 (2H, md, J=8.7 Hz), 7.30 (2H, md, J=8.7 Hz), 6.64 (2H, s br), 6.59 (1H, s), 6.05 (2H, s), 4.11 (2H, s br), 3.77 (6H, s), 3.03 (2H, br), 2.94 (3H, s br), 2.05 (2H, m br), 1.97 (2H, m).

¹³C NMR (DMSO), δ (ppm): 167.2, 160.7, 151, 149.1, 139.8, 137.9, 135.6, 131.8, 126, 122.5, 114.7, 108.8, 100.6, 59.6, 55.4, 52.6, 51.1, 32, 25.7.

### Example 20

### N-(1-(3-methoxybenzyl)piperidin-4-yl)-N-methyl-4-(3-(sulfamoylamino)phenyl)-1H-imidazole-1-carboxamide dimaleate

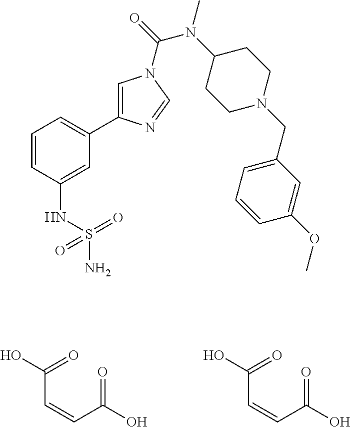 [SMILES: COc1cccc(CN2CCC(N(C)C(=O)n3cnc(-c4cccc(NS(N)(=O)=O)c4)c3)CC2)c1 ; O=C(O)/C=C\C(=O)O ; O=C(O)/C=C\C(=O)O]

The title compound was prepared by analogous manner to Example 19 from N-(1-(3-methoxybenzyl)piperidin-4-yl)-N-methyl-4-(3-(sulfamoylamino)phenyl)-1H-imidazole-1-carboxamide (Example 3 step 1).

Appearance: white solid.

¹H NMR (DMSO), δ (ppm): 9.55 (1H, s), 8.14 (1H, s br), 7.94 (1H, s br), 7.64 (1H, t, J=1.9 Hz), 7.44 (1H, td, J=1.1, 7.7 Hz), 7.41 (1H, t, J=7.9 Hz), 7.27 (1H, t, J=7.8 Hz), 7.15 (2H, s br), 7.11-7.02 (4H, m), 6.16 (4H, s), 4.25 (2H, s br), 4.15 (1H, br), 3.45 (2H, m), 3.13 (2H, m), 2.93 (3H, s), 2.08 (2H, m), 2.0 (2H, m).

¹³C NMR (DMSO), δ (ppm): 167, 159.5, 151.1, 140.7, 139.9, 137.6, 133.9, 133, 131.3, 130.2, 129.1, 123.2, 118.6, 116.9, 116.8, 115, 114.5, 114.4, 59.2, 55.3, 52.3, 50.8, 31.9, 25.3.

### Example 21

### N-(1-benzylpiperidin-4-yl)-N-methyl-4-(3-ureidophenyl)-1H-imidazole-1-carboxamide maleate

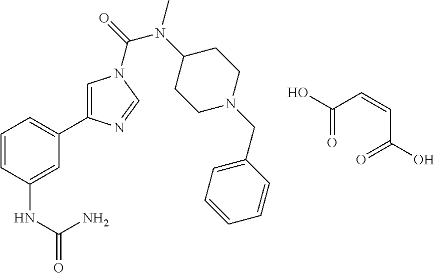 [SMILES: CN(C(=O)n1cnc(-c2cccc(NC(N)=O)c2)c1)C1CCN(Cc2ccccc2)CC1 ; O=C(O)/C=C\C(=O)O]

The title compound was prepared by analogous manner to Example 19 in methanol from: N-(1-benzylpiperidin-4-yl)-N-methyl-4-(3-ureidophenyl)-1H-imidazole-1-carboxamide (Example 16 step 1).

Appearance: off-white solid.

¹H NMR (DMSO), δ (ppm): 8.58 (1H, s br), 8.12 (1H, s br), 7.89 (2H, m), 7.48 (5H, m br), 7.35 (1H, td, J=1.2, 7.6 Hz), 7.30 (1H, dddd, J=1.0, 2.0, 8.0 Hz), 7.22 (1H, t, J=7.8 Hz), 6.05 (2H, s), 5.86 (2H, s br), 4.34-4.0 (3H, 2 s br), 3.04 (2H, br), 2.93 (3H, s), 2.04 (2H, m br), 1.97 (2H, m br).

¹³C NMR (DMSO), δ (ppm): 167.2, 156, 151.1, 140.9, 140.9, 137.6, 135.5, 133.6, 131, 129.4, 128.9, 128.9, 117.9, 116.7, 114.3, 114.2, 59.5, 52.6, 50.8, 31.9, 25.5.

### Example 22

### N-(1-benzylpiperidin-4-yl)-N-methyl-4-(3-ureidophenyl)-1H-imidazole-1-carboxamide methanesulfonate

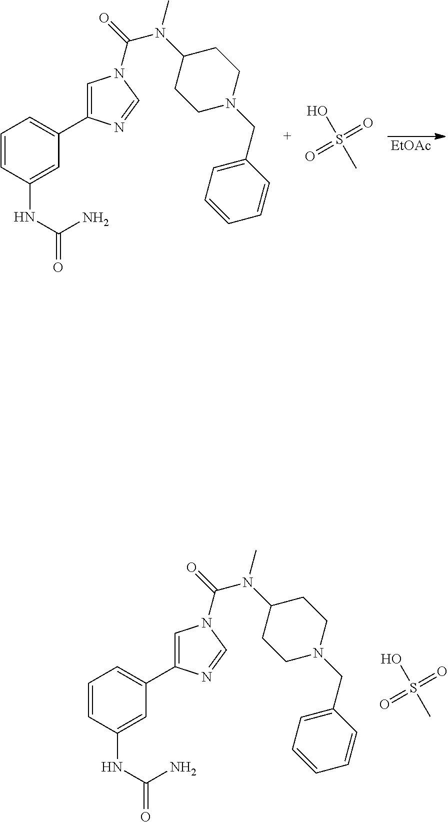

A mixture of N-(1-benzylpiperidin-4-yl)-N-methyl-4-(3-ureidophenyl)-1H-imidazole-1-carboxamide (200 mg, 0.462 mmol) (Example 16 step 1) and methanesulfonic acid (44.4 mg, 0.462 mmol) was refluxed in ethyl acetate (5 mL) for 30 min. The reaction mixture was cooled to room temperature and the precipitate was filtered, washed with ether and dried to give white solid. (Yield: 176 mg, 72%).

¹H NMR (DMSO), δ (ppm): 9.42 (1H, s br), 8.63 (1H, s), 8.25 (1H, s), 7.92 (1H, s), 7.90 (1H, t, J=1.8 Hz), 7.5 (5H, m), 7.35 (1H, td, J=1.0, 7.7 Hz), 7.31 (1H, ddd, J=1.0, 2.0, 8.0 Hz), 7.23 (1H, t, J=7.8 Hz), 5.9 (2H, s br), 4.30 (2H, d, J=5.0 Hz), 4.15 (1H, s br), 3.45 (2H, d, J=11.5 Hz), 3.15 (2H, m), 2.93 (3H, s), 2.37 (3H, s), 2.12 (2H, dq, J=2.5, 12.5 Hz), 2.0 (2H, d, J=13.5 Hz).

¹³C NMR (DMSO), δ (ppm): 156, 150.9, 141, 140.4, 137.6, 133.1, 131.3, 129.7, 129.7, 129, 128.9, 117.9, 116.9, 114.4, 114.3, 59.1, 52.2, 50.6, 39.8, 31.9, 25.2.

### Example 23

### N-(1-(3-methoxybenzyl)piperidin-4-yl)-N-methyl-4-(3-(sulfamoylamino)phenyl)-1H-imidazole-1-carboxamide methanesulfonate

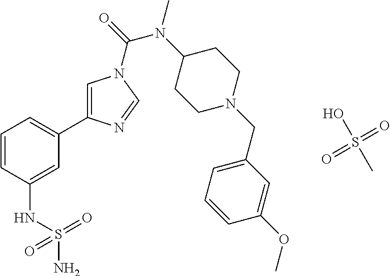

The title compound was prepared by analogous manner to Example 22 in methanol from: N-(1-(3-methoxybenzyl)piperidin-4-yl)-N-methyl-4-(3-(sulfamoylamino)phenyl)-1H-imidazole-1-carboxamide (Example 3 step 2).

Appearance: white solid.

¹H NMR (DMSO), δ (ppm): 10.55 (1H, s br), 9.67 (1H, s br), 8.75 (1H, s br), 8.15 (1H, s), 7.61 (1H, t, J=1.7 Hz), 7.45 (1H, d, J=8.0 Hz), 7.37 (1H, t, J=8.0 Hz), 7.34 (1H, t, J=7.9 Hz), 7.26 (1H, t, J=1.8 Hz), 7.22 (2H, m br), 7.11 (1H, d, J=7.8 Hz), 7.09 (1H, ddd, J=0.8, 2.1, 8.2 Hz), 7.03 (1H, dd, J=2.8, 8.5 Hz), 4.24 (2H, d, J=4.9 Hz), 4.19 (1H, br), 3.79 (3H, s), 3.41 (2H, d, J=11.2 Hz), 3.11 (2H, m), 2.96 (3H, s), 2.37 (3H, s), 2.31 (2H, dq, J=3.0, 12.5 Hz), 1.97 (2H, d, J=12.3 Hz).

¹³C NMR (DMSO), δ (ppm): 159.4, 150, 140.1, 137.8, 137.5, 131.3, 131.2, 130, 129.4, 123.3, 118.9, 117.8, 116.7, 115.4, 115.2, 114.3, 58.9, 55.3, 52.3, 50.4, 39.8, 31.8, 24.8.

### Example 24

### N-(1-(4-hydroxyphenyl)piperidin-4-yl)-N-methyl-4-(3-(methylsulfonamido) phenyl)-1H-imidazole-1-carboxamide hydrochloride

### Step1: N-(1-(4-methoxyphenyl)piperidin-4-yl)-N-methyl-4-(3-(methylsulfonamido) phenyl)-1H-imidazole-1-carboxamide

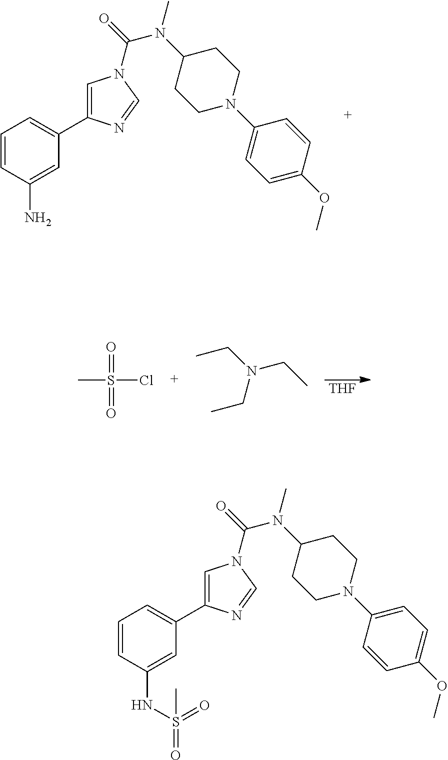

A solution of Intermediate 15 (250 mg, 0.617 mmol) in anhydrous tetrahydrofuran (10 mL), under nitrogen, was treated with triethylamine (0.128 mL, 0.925 mmol), followed by dropwise addition of methanesulfonyl chloride (0.072 mL, 0.925 mmol). The mixture was stirred at room temperature overnight. The solvent was evaporated and the residue was purified by column chromatography (dichloromethane/methanol 9:1) to yield an off-white solid. (Yield: 128 mg, 43%).

### Step2: N-(1-(4-hydroxyphenyl)piperidin-4-yl)-N-methyl-4-(3-(methylsulfonamido)phenyl)-1H-imidazole-1-carboxamide

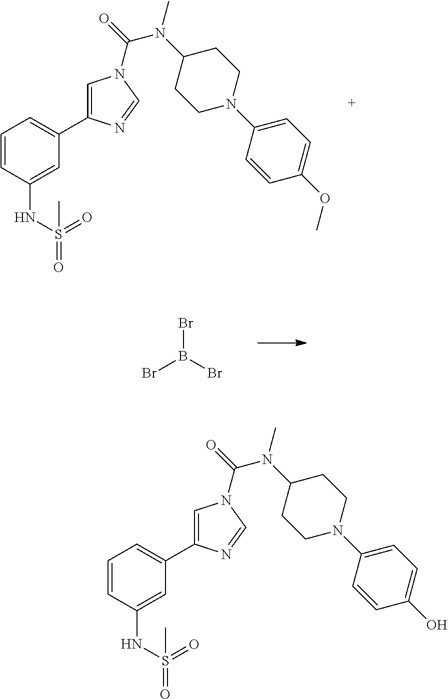

To a −78° C. cold suspension of N-(1-(4-methoxyphenyl)piperidin-4-yl)-N-methyl-4-(3-(methylsulfonamido)phenyl)-1H-imidazole-1-carboxamide (120 mg, 0.248 mmol) in dry dichloromethane (7.30 mL) was added boron tribromide (0.0706 mL, 0.744 mmol). The reaction was allowed to stir in the cold for 15 min and then at room temperature overnight. Then, the reaction was quenched with crushed ice, stirred for 2 h, and then transferred into separatory funnel; partitioned between water and dichloromethane/isopropanol 7:3, and the aqueous phase was further extracted into dichloromethane/isopropanol 7:3. The combined organic layers were dried over anhydrous Na₂SO₄, filtered through a pad of celite, and concentrated. The residue was purified by column chromatography (dichloromethane/methanol 9:1) to yield an off-white solid. (Yield: 65 mg, 56%).

### Step3: N-(1-(4-hydroxyphenyl)piperidin-4-yl)-N-methyl-4-(3-(methylsulfonamido)phenyl)-1H-imidazole-1-carboxamide hydrochloride

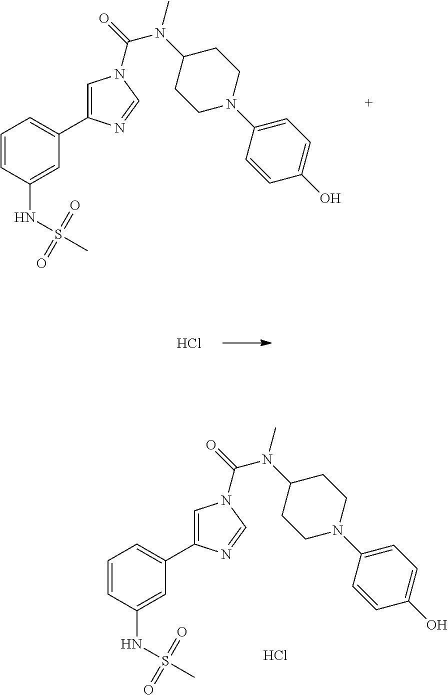 [SMILES: CN(C(=O)n1cnc(-c2cccc(NS(C)(=O)=O)c2)c1)C1CCN(c2ccc(O)cc2)CC1 ; Cl ; CN(C(=O)n1cnc(-c2cccc(NS(C)(=O)=O)c2)c1)C1CCN(c2ccc(O)cc2)CC1 ; Cl]

A solution of N-(1-(4-hydroxyphenyl)piperidin-4-yl)-N-methyl-4-(3-(methylsulfonamido)phenyl)-1H-imidazole-1-carboxamide (65 mg, 0.138 mmol) in ethyl acetate (2 ml) was treated with 2M hydrogen chloride solution in diethylether (0.069 ml, 0.138 mmol). The reaction mixture was stirred for 30 min and filtered. The filter cake was washed with methanol and ethyl ether and dried under vacuum to yield an off-white powder. (Yield: 61 mg, 87%).

¹H NMR (DMSO), δ (ppm): 12.89 (1H, s br), 10.10 (1H, s br), 9.87 (1H, s), 8.59 (1H, s), 8.14 (1H, s), 7.73 (1H, t, J=1.8 Hz), 7.71 (2H, d, J=8.6 Hz), 7.60 (1H, md, J=7.8 Hz), 7.39 (1H, t, J=7.9 Hz), 7.16 (1H, md, J=8.0 Hz), 6.90 (2H, d, J=9.0 Hz), 4.45 (1H, s br), 3.80 (2H, m), 3.54 (2H, m), 3.03 (3H, s), 3.02 (3H, s), 2.70 (2H, m), 2.03 (2H, m).

¹³C NMR (DMSO), δ (ppm): 158.2, 150.5, 138.9, 138.4, 137.7, 133.8, 132.7, 129.7, 122.7, 120.8, 119.3, 116.4, 116.2, 115.4, 54.7, 51.4, 31.5, 25.3.

### Example 25

### N-(1-(4-hydroxyphenyl)piperidin-4-yl)-N-methyl-4-(3-ureidophenyl)-1 H-imidazole-1-carboxamide hydrochloride

### Step1: 4-(3-aminophenyl)-N-(1-(4-hydroxyphenyl)piperidin-4-yl)-N-methyl-1H-imidazole-1-carboxamide

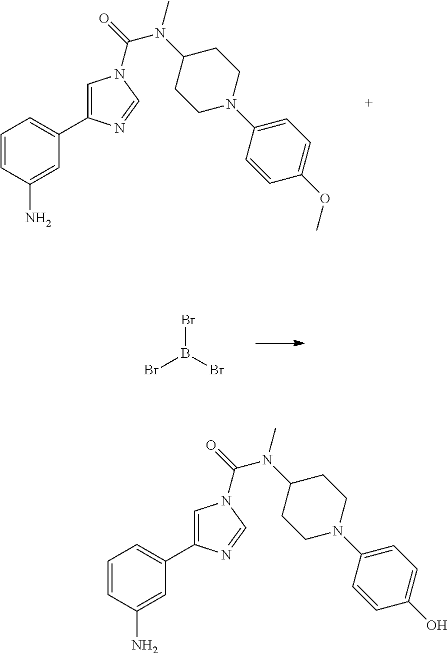

To a −78° C. cold suspension of Intermediate 15 (250 mg, 0.617 mmol) in dichloromethane (18 mL) was added boron tribromide (0.176 mL, 1.855 mmol) dropwise. The reaction was allowed to stir in the cold for 15 min and then at room temperature overnight. Then, the reaction was quenched with crushed ice, stirred for 2 h. The mixture was transferred into a separatory funnel; partitioned between water and dichloromethane/isopropanol 7:3, and the aqueous phase was further extracted into dichloromethane/isopropanol 7:3. The combined organic layers were dried over anhydrous Na₂SO₄, filtered through a pad of celite, and concentrated. The residue was purified by column chromatography (dichloromethane/methanol 9:1) to yield an off-white solid. (Yield: 228 mg, 94%).

### Step2 N-(1-(4-hydroxyphenyl)piperidin-4-yl)-N-methyl-4-(3-ureidophenyl)-1H-imidazole-1-carboxamide

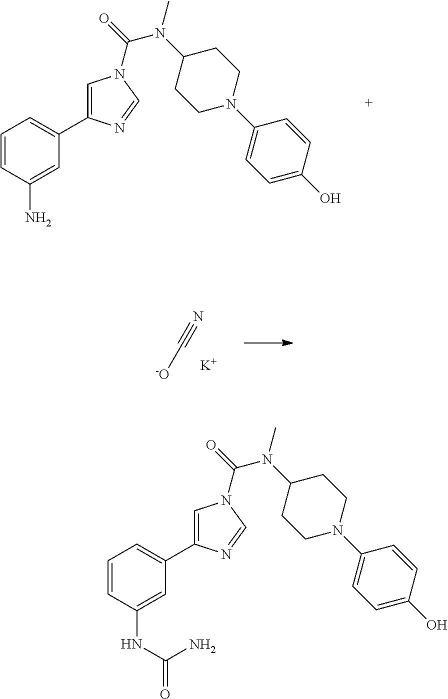

An aqueous solution (1 mL) of potassium cyanate (94 mg, 1.165 mmol) was added dropwise, at 0° C., to a solution of 4-(3-aminophenyl)-N-(1-(4-hydroxyphenyl)piperidin-4-yl)-N-methyl-1H-imidazole-1-carboxamide (228 mg, 0.582 mmol) in acetic acid (2.5 mL). The reaction mixture was stirred at 0° C. for 2 h, followed by another 4 h at room temperature. The reaction mixture was diluted with 50 mL of water and transferred to a separating funnel. the acidic solution was neutralized with saturated NaHCO₃ solution and extracted with 3×10 mL of ethyl acetate. The organic phases were dried over MgSO₄ and concentrated. The crude mixture was purified by column chromatography and the resulting oil was crystallized from isopropanol to yield a white solid. (Yield: 151 mg, 60%).

### Step3 N-(1-(4-hydroxyphenyl)piperidin-4-yl)-N-methyl-4-(3-ureidophenyl)-1H-imidazole-1-carboxamide hydrochloride

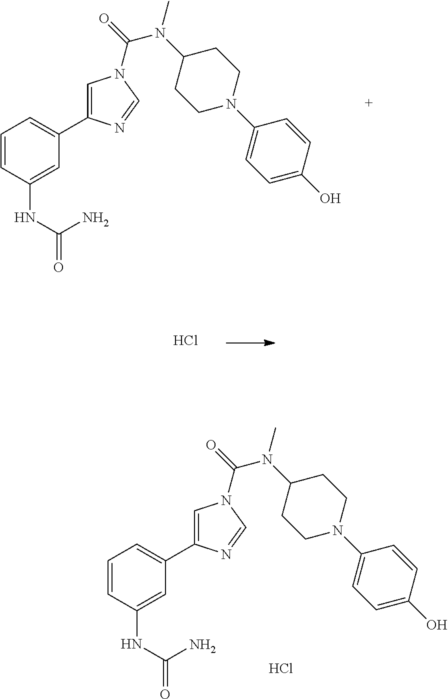 [SMILES: CN(C(=O)n1cnc(-c2cccc(NC(N)=O)c2)c1)C1CCN(c2ccc(O)cc2)CC1 ; Cl ; CN(C(=O)n1cnc(-c2cccc(NC(N)=O)c2)c1)C1CCN(c2ccc(O)cc2)CC1 ; Cl]

A solution of N-(1-(4-hydroxyphenyl)piperidin-4-yl)-N-methyl-4-(3-ureidophenyl)-1H-imidazole-1-carboxamide (145 mg, 0.334 mmol) in ethyl acetate (2 ml) and methanol (2 mL) was treated with 2M hydrogen chloride solution in diethylether (0.167 mL, 0.334 mmol). The reaction mixture was stirred for 30 min and filtered. The filter cake was washed with methanol and ethyl ether and dried under vacuum to yield an off-white powder. (Yield: 89 mg, 57%).

¹H NMR (DMSO), δ (ppm): 12.73 (1H, s br), 10.10 (1H, s br), 8.77 (1H, s), 8061 (1H, d br), 8.07 (1H, s), 7.91 (1H, s), 7.69 (2H, d, J=7.7 Hz), 7.38 (1H, md, J=7.6 Hz), 7.35 (1H, md, J=8.1 Hz), 7.27 (1H, t, J=8.0 Hz), 6.90 (2H, d, J=9.0 Hz), 5.92 (1H, br), 4.44 (1H, s br), 3.76 (2H, m), 3.02 (3H, s), 2.66 (2H, m), 2.04 (2H, m).

¹³C NMR (DMSO), δ (ppm): 158.2, 156.1, 150.1, 141.2, 137.9, 137.5, 133.8, 130.8, 129.1, 122.7, 118.3, 117.7, 116.2, 115.2, 114.6, 54.7, 51.5, 31.6, 25.3.

### Example 26

### N-(1-(3,4-dimethoxybenzyl)piperidin-4-yl)-N-methyl-4-(3-ureidophenyl)-1H-imidazole-1-carboxamide hydrochloride

### Step1: N-(1-(3, 4-dimethoxybenzyl)piperidin-4-yl)-N-methyl-4-(3-ureidophenyl)-1H-imidazole-1-carboxamide

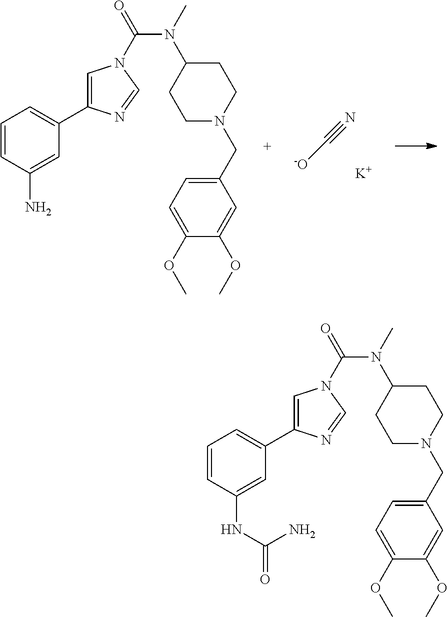

In a 50 mL pear flask was placed Intermediate 9 (700 mg, 1.557 mmol), water (1 mL) and hydrogen chloride (0.779 mL, 1.557 mmol) to give a pale yellow suspension. The suspension was cooled to 0° C. and potassium cyanate (152 mg, 1.869 mmol) was added, in portions. The mixture was stirred at room temperature overnight. Another portion of hydrogen chloride (0.779 mL, 1.557 mmol) and potassium cyanate (152 mg, 1.869 mmol) was added and the mixture was stirred at room temperature for 24 h. The reaction mixture was filtered, the filter cake was washed with water and purified by column chromatography (dichloromethane/methanol 9:1) to yield a white foam. (Yield: 479 mg, 56%).

### Step2: N-(1-(3, 4-dimethoxybenzyl)piperidin-4-yl)-N-methyl-4-(3-ureidophenyl)-1H-imidazole-1-carboxamide hydrochloride

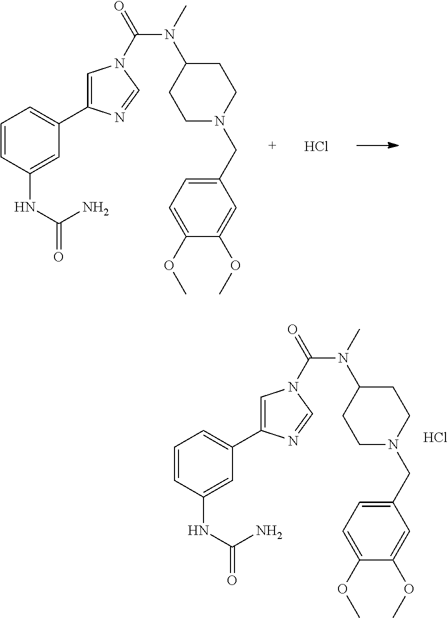 [SMILES: COc1ccc(CN2CCC(N(C)C(=O)n3cnc(-c4cccc(NC(N)=O)c4)c3)CC2)cc1OC ; Cl ; COc1ccc(CN2CCC(N(C)C(=O)n3cnc(-c4cccc(NC(N)=O)c4)c3)CC2)cc1OC ; Cl]

In a 50 mL pear flask N-(1-(3,4-dimethoxybenzyl)piperidin-4-yl)-N-methyl-4-(3-ureidophenyl)-1H-imidazole-1-carboxamide (479 mg, 0.972 mmol) was dissolved in hot ethyl acetate (10 mL). The solution was cooled to 0° C. and hydrogen chloride 2M solution in diethyl ether (3.65 mL, 7.29 mmol) was added, dropwise. The suspension was stirred at 0° C. for 10 min, then was allowed to warm to room temperature and stirred for 2 h. The reaction mixture was filtered, filter cake was washed with diethyl ether, and crystallized from dichloromethane/isopropanol to yield an off-white solid. (Yield: 335 mg, 55%).

¹H NMR (DMSO), δ (ppm): 11.22 (1H, s), 8.93 (2H, m), 8.14 (1H, s), 7.90 (1 h, t, J=1.8 Hz), 7.41 (1H, d, J=1.8 Hz), 7.38 (1H, d, J=1.6 Hz), 7.37 (1H, d, J=1.7 Hz), 7.30 (1H, dd, J=7.4, 8.3 Hz), 7.05 (1H, dd, J=1.6, 8.1 Hz), 6.99 (1H, d, J=8.3 Hz), 5.92 (br), 4.20 (1H, br), 4.17 (2H, d, J=5.0 Hz), 3.80 (3H, s), 3.77 (3H, s), 3.37 (2H, d, J=11.3 Hz), 3.05 (2H, m), 2.96 (3H, s), 2.41 (2H, dq, J=3.1, 12.7 Hz), 1.95 (2H, d, J=12.5 Hz)

¹³C NMR (DMSO), δ (ppm): 156, 150.1, 149.5, 148.6, 141.1, 138.3, 137.5, 131.1, 129.1, 123.8, 121.9, 118.2, 117.5, 114.9, 114.6, 114.5, 111.4, 58.8, 55.6, 55.5, 52.3, 50.1, 31.5, 24.7

### Example 27

### 4-(1-((1-(3,5-dimethoxybenzyl)piperidin-4-yl)(methyl)carbamoyl)-1H-imidazol-4-yl)phenyl sulfamate methanesulfonate

### Step 1: 4-(1-((1-(3,5-dimethoxybenzyl)piperidin-4-yl)(methyl)carbamoyl)-1H-imidazol-4-yl)phenyl sulfamate

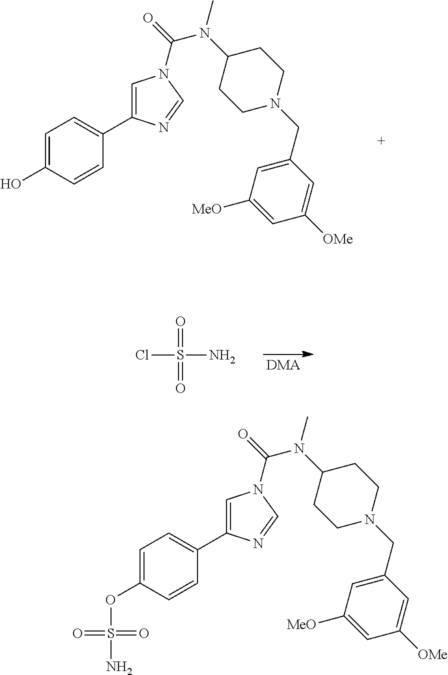

To a turbid solution of N-(1-(3,5-dimethoxybenzyl)piperidin-4-yl)-4-(4-hydroxyphenyl)-N-methyl-1H-imidazole-1-carboxamide (Example 8. Step1) (153 mg, 0.340 mmol) in N,N-dimethylacetamide (1.4 mL) was added sulfamoyl chloride (157 mg, 1.358 mmol) and stirred the reaction mixture at room temperature. After 48 h. the mixture was loaded on a column and eluted (silica gel H; gradient dichloromethane/methanol 49:1, then dichloromethane/methanol/ammonium hydroxide (25%) 95:5:0.1). Repeated column chromatography (silica gel H; dichloromethane/methanol 9:1) to afford the title product. (Yield: 108 mg, 61%).

### Step2: 4-(1-((1-(3,5-dimethoxybenzyl)piperidin-4-yl)(methyl)carbamoyl)-1H-imidazol-4-yl)phenyl sulfamate methanesulfonate

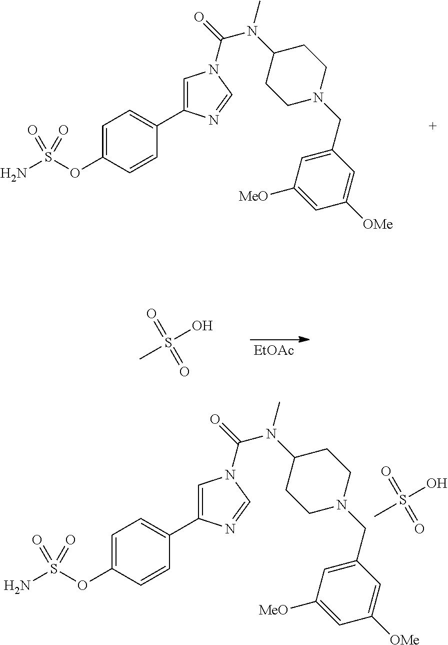

4-(1-((1-(3,5-dimethoxybenzyl)piperidin-4-yl)(methyl)carbamoyl)-1H-imidazol-4-yl)phenyl sulfamate (200 mg, 0.378 mmol) and methanesulfonic acid (0.025 mL, 0.378 mmol) were refluxed in ethyl acetate (5 ml) for 15 min. The reaction mixture was cooled to room temperature and the obtained precipitate was filtered and washed with diethyl ether. The precipitate was dried under vacuum to yield a white solid. (Yield: 221 mg, 94%).

¹H NMR (DMSO), δ (ppm): 9.38 (1H, s), 8.20 (1H, s), 8.06 (1H, s), 8.03 (2H, s), 7.92 (2H, md, J=8.7 Hz), 7.30 (2H, md, J=8.7 Hz), 6.69 (2H, d, J=2.2 Hz), 6.61 (1H, t, J=2.2 Hz), 4.21 (2H, d, J=5.0 Hz), 4.16 (1H, s br), 3.78 (6H, s), 3.45 (2H, m), 3.13 (2H, m), 2.94 (3H, s), 2.35 (3H, s), 2.14 (2H, dq, J=2.5, 13.0 Hz), 2.0 (2H, d, J=12.8 Hz).

¹³C NMR (DMSO), δ (ppm): 160.7, 150.3, 149.6, 137.9, 137.8, 131.7, 129.8, 126.4, 122.6, 115.3, 109.1, 101, 59.2, 55.4, 52.4, 50.8, 39.8, 32, 25.1.

### Example 28

### 4-(1-((1-(4-hydroxyphenyl)piperidin-4-yl)(methyl)carbamoyl)-1H-imidazol-4-yl)phenyl sulfamate hydrochloride

### Step1: 4-(4-(benzyloxy)phenyl)-N-(1-(4-methoxyphenyl)piperidin-4-yl)-N-methyl-1H-imidazole-1-carboxamide

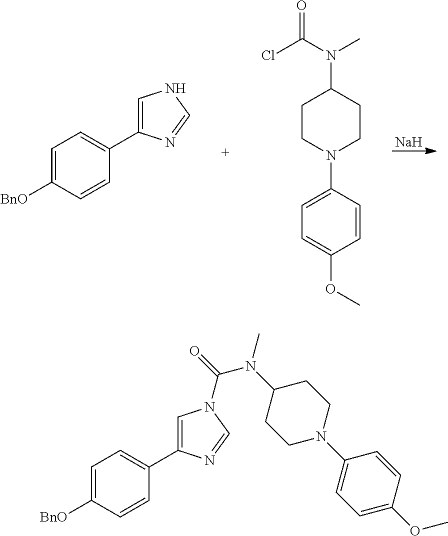

A cold solution of Intermediate 17 (500 mg, 1.998 mmol) in tetrahydrofuran (10 mL) was treated portionwise with sodium hydride (60% in oil dispersion) (88 mg, 2.197 mmol). The reaction was stirred for 30 min and then Intermediate 6 (678 mg, 2.397 mmol) was added in one portion. The reaction was allowed to warm up to room temperature and stirred overnight. The reaction was quenched with small amount of ice, transferred to a separatory funnel and the aqueous mixture was extracted with dichloromethane. The organic phase was dried over MgSO₄ and concentrated. The crude oil was purified on silica gel column (dichloromethane to dichloromethane/methanol 95:5). Recrystallization from isopropanol yielded a white solid. (Yield: 505 mg, 51%).

### Step2: 4-(4-hydroxyphenyl)-N-(1-(4-methoxyphenyl)piperidin-4-yl)-N-methyl-1H-imidazole-1-carboxamide

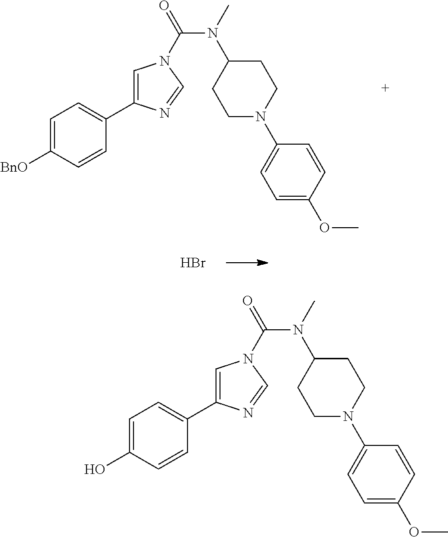 [SMILES: COc1ccc(N2CCC(N(C)C(=O)n3cnc(-c4ccc(OCc5ccccc5)cc4)c3)CC2)cc1 ; Br ; COc1ccc(N2CCC(N(C)C(=O)n3cnc(-c4ccc(O)cc4)c3)CC2)cc1]

To a cold suspension of 4-(4-(benzyloxy)phenyl)-N-(1-(4-methoxyphenyl)piperidin-4-yl)-N-methyl-1H-imidazole-1-carboxamide (0.5 g, 1.007 mmol) in dichloromethane (8 mL) was added hydrogen bromide (33% in acetic acid) (1.243 mL, 7.55 mmol) dropwise. The mixture was allowed to stir at room temperature for 2 h, Then, the reaction was quenched with water and neutralized upon careful addition of saturated aqueous solution of Na₂CO₃. The mixture was extracted with dichloromethane/isopropanol 7/3 until no further material could be extracted. The combined organic layers were dried over anhydrous Na₂SO₄, filtered and concentrated to yield a white solid. (Yield: 355 mg, 87%).

### Step3: 4-(1-((1-(4-methoxyphenyl)piperidin-4-yl)(methyl)carbamoyl)-1H-imidazol-4-yl)phenyl sulfamate

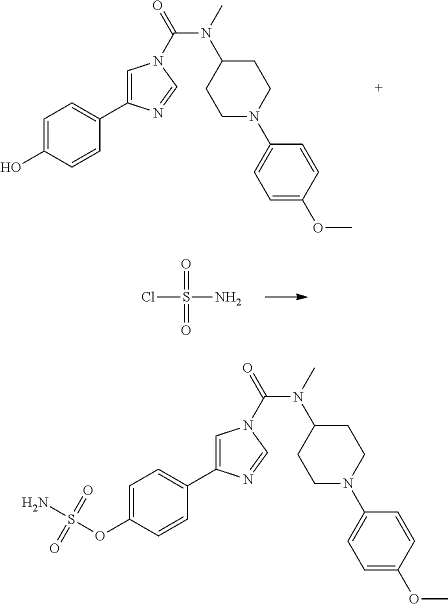 [SMILES: COc1ccc(N2CCC(N(C)C(=O)n3cnc(-c4ccc(O)cc4)c3)CC2)cc1 ; NS(=O)(=O)Cl ; COc1ccc(N2CCC(N(C)C(=O)n3cnc(-c4ccc(OS(N)(=O)=O)cc4)c3)CC2)cc1]

To a solution of 4-(4-hydroxyphenyl)-N-(1-(4-methoxyphenyl)piperidin-4-yl)-N-methyl-1H-imidazole-1-carboxamide (350 mg, 0.861 mmol) in N,N-dimethylacetamide (4 mL) was added sulfamoyl chloride (398 mg, 3.44 mmol) and stirred the reaction mixture at room temperature overnight. The reaction mixture was quenched with saturated aqueous solution of NaHCO₃ and the resultant precipitate was filtered, washed with water, and dried under vacuum to yield an off-white solid. (Yield: 401 mg, 96%).

### Step4: 4-(1-((1-(4-hydroxyphenyl)piperidin-4-yl)(methyl)carbamoyl)-1H-imidazol-4-yl)phenyl sulfamate

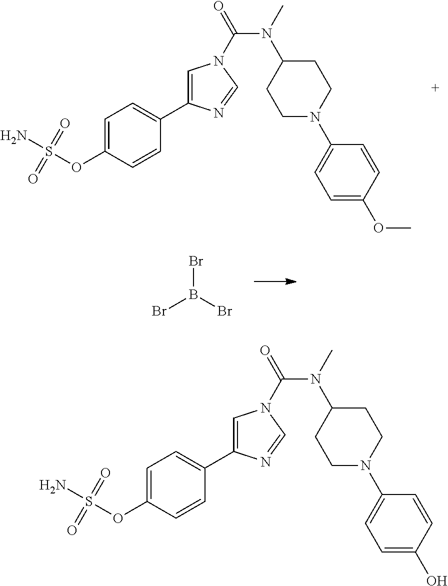

To a suspension of 4-(1-((1-(4-methoxyphenyl)piperidin-4-yl)(methyl)carbamoyl)-1H-imidazol-4-yl)phenyl sulfamate (395 mg, 0.814 mmol) in dry dichloromethane (24 mL) was added dropwise boron tribromide (0.271 mL, 2.86 mmol) at −78° C. After stirring in the cold for 1 h the reaction was allowed to stir at room temperature overnight. Once completed, the reaction mixture was cooled in crushed ice/water bath and quenched it with crushed ice. The resultant white solid was purified by column chromatography (dichloromethane/methanol 9:1) to yield a white solid. Recrystallization from isopropanol afforded the title compound. (Yield: 122 mg, 32%).

### Step5: 4-(1-((1-(4-hydroxyphenyl)piperidin-4-yl)(methyl)carbamoyl)-1H-imidazol-4-yl)phenyl sulfamate hydrochloride

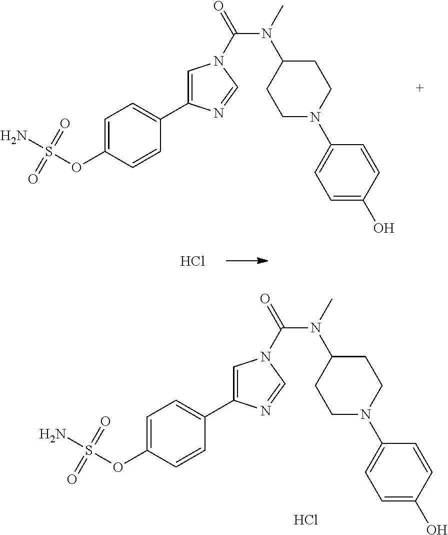 [SMILES: CN(C(=O)n1cnc(-c2ccc(OS(N)(=O)=O)cc2)c1)C1CCN(c2ccc(O)cc2)CC1 ; Cl ; CN(C(=O)n1cnc(-c2ccc(OS(N)(=O)=O)cc2)c1)C1CCN(c2ccc(O)cc2)CC1 ; Cl]

A solution of 4-(1-((1-(4-hydroxyphenyl)piperidin-4-yl)(methyl)carbamoyl)-1H-imidazol-4-yl)phenyl sulfamate (115 mg, 0.244 mmol) in ethyl acetate (3 mL) was treated with hydrogen chloride 2M in diethyl ether (0.122 mL, 0.244 mmol). The reaction mixture was stirred for 30 min and filtered. The filter cake was washed with methanol and diethyl ether and dried under vacuum to yield an off-white powder. (Yield: 61 mg, 49%).

¹H NMR (DMSO), δ (ppm): 12.57 (1H, s br), 10.09 (1H, s br), 8.26 (1H, s), 8.10 (1H, s), 8.03 (2H, s), 7.93 (2H, md, J=8.7 Hz), 7.67 (2H, m), 7.31 (2H, md, J=8.7 Hz), 6.89 (2H, d br, J=7.2 Hz), 4.43 (1H, s), 3.77 (2 h, m), 3.55 (2H, d, J=11.5 Hz), 3.01 (3H, s), 2.63 (2H, br), 2.03 (2H, d, J=11.8 Hz).

¹³C NMR (DMSO), δ (ppm): 158.3, 151, 149.3, 139.3, 137.9, 133.7, 131.3, 126.1, 122.7, 122.5, 116.2, 114.9, 54.7, 51.4, 31.6, 25.5.

### Example 29

### 3-(1-((1-(4-bromo-3-hydroxyphenyl)piperidin-4-yl)(methyl)carbamoyl)-1 H-imidazol-4-yl)phenyl sulfamate hydrochloride

### Step1: 1-(3-(benzyloxy)phenyl)-2-bromoethanone

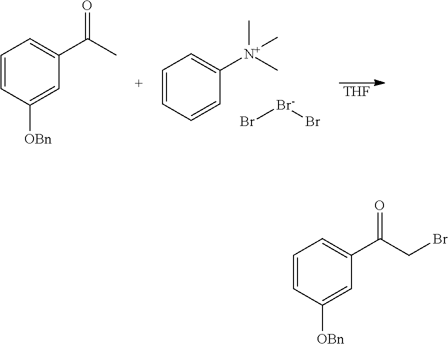

In a 500 mL round-bottomed flask was placed 1-(3-(benzyloxy)phenyl)ethanone (16.62 g, 73.5 mmol) in tetrahydrofuran (160 mL) at 0° C., under inert atmosphere, to give a colorless solution. A solution of phenyltrimethylammonium tribromide (30.4 g, 81 mmol) in tetrahydrofuran (160 mL) was added to the above solution, dropwise. The resultant orange suspension was allowed to warm up to room temperature and stirred for 6 h. The insoluble material was then filtered off, the filter cake was washed with tetrahydrofuran. The filtrate was evaporated and the orange oil was crystallized from isopropanol to yield the title product as off-white crystals. (Yield: 19.26 g, 86%).

### Step2: 4-(3-(benzyloxy)phenyl)-1H-imidazole

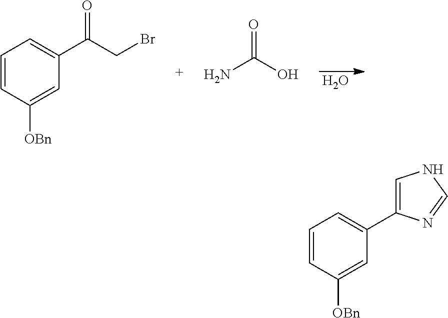

1-(3-(benzyloxy)phenyl)-2-bromoethanone (19.26 g, 63.1 mmol) was suspended in formamide (31.1 mL, 783 mmol) and water (2.2 mL). The heterogeneous mixture was heated at 140° C. for 6 h. Then, the reaction was cooled to room temperature, and quenched with water. A brown solid precipitated, which was filtered off and the filtrate was basified to pH 12 with a 20% NaOH aqueous solution. The aqueous phase was extracted with dichloromethane/isopropanol 7:3. The organic phase was dried over MgSO₄ and concentrated. The residue was purified by column chromatography (dichloromethane/methanol 9:1) to yield a light orange solid. (Yield: 8.654 g, 55%).

### Step3: 4-(3-(benzyloxy)phenyl)-N-(1-(4-bromo-3-methoxyphenyl)piperidin-4-yl)-N-methyl-1 H-imidazole-1-carboxamide

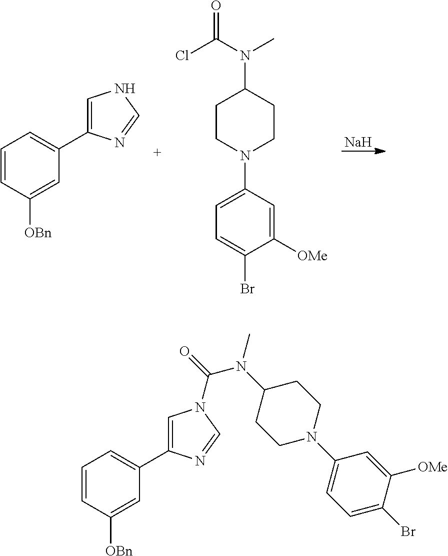

A cold solution of 4-(3-(benzyloxy)phenyl)-1H-imidazole (1 g, 4.00 mmol) in tetrahydrofuran (15 mL) was treated with sodium hydride (60% in oil dispersion) (0.208 g, 5.19 mmol) and stirred for 30 min. Then, Intermediate 18 (1.589 g, 4.39 mmol) was added in one portion and the reaction was allowed to warm up to room temperature and stirred overnight. Then, the reaction was transferred into a separatory funnel, diluted with 100 mL of dichloromethane and the organic layer was washed with aqueous HCl 1N and water. The organic phase was dried over MgSO₄, concentrated and the residue was crystallized from hot isopropanol to yield an off-white solid. (Yield: 1.203 g, 52%).

### Step4: N-(1-(4-bromo-3-methoxyphenyl)piperidin-4-yl)-4-(3-hydroxyphenyl)-N-methyl-1H-imidazole-1-carboxamide hydrobromide

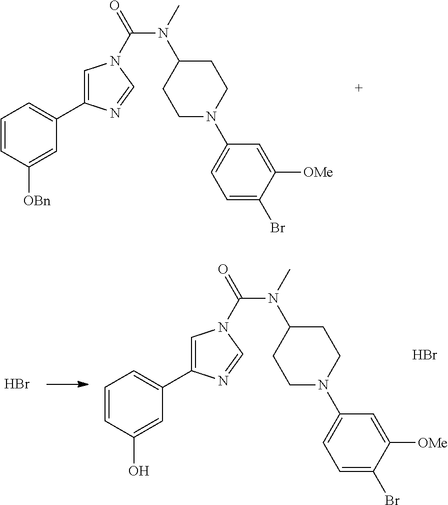

To a cold suspension of 4-(3-(benzyloxy)phenyl)-N-(1-(4-bromo-3-methoxyphenyl)piperidin-4-yl)-N-methyl-1H-imidazole-1-carboxamide (0.5 g, 0.869 mmol) in dichloromethane (7 mL) added dropwise hydrogen bromide (33% in acetic acid) (1.072 mL, 6.52 mmol). The reaction was allowed to stir at room temperature until its completion. Then, the resultant precipitate was filtered off, washed with dichloromethane and dried under vacuum to yield the title product as a white solid. (Yield: 0.503 g, 102%).

### Step5: 3-(1-((1-(4-bromo-3-methoxyphenyl)piperidin-4-yl)(methyl)carbamoyl)-1H-imidazol-4-yl)phenyl sulfamate

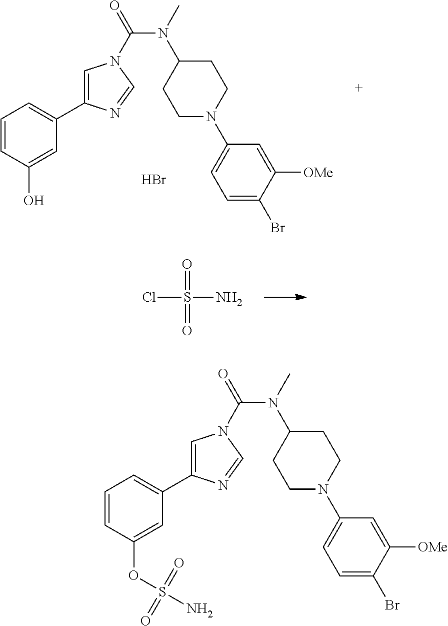 [SMILES: COc1cc(N2CCC(N(C)C(=O)n3cnc(-c4cccc(O)c4)c3)CC2)ccc1Br ; Br ; NS(=O)(=O)Cl ; COc1cc(N2CCC(N(C)C(=O)n3cnc(-c4cccc(OS(N)(=O)=O)c4)c3)CC2)ccc1Br]

To a solution of N-(1-(4-bromo-3-methoxyphenyl)piperidin-4-yl)-4-(3-hydroxyphenyl)-N-methyl-1H-imidazole-1-carboxamide hydrobromide (496 mg, 0.876 mmol) in N,N-dimethylacetamide (4.735 mL) was added sulfamoyl chloride (304 mg, 2.63 mmol) and stirred the reaction mixture at room temperature overnight. Thereupon, the reaction mixture was quenched with saturated aqueous solution of NaHCO₃ and the obtained precipitate was filtered off, washed with water, and dried under vacuum to yield an off-white solid. (Yield: 217 mg, 44%).

### Step6: 3-(1-((1-(4-bromo-3-hydroxyphenyl)piperidin-4-yl)(methyl)carbamoyl)-1H-imidazol-4-yl)phenyl sulfamate

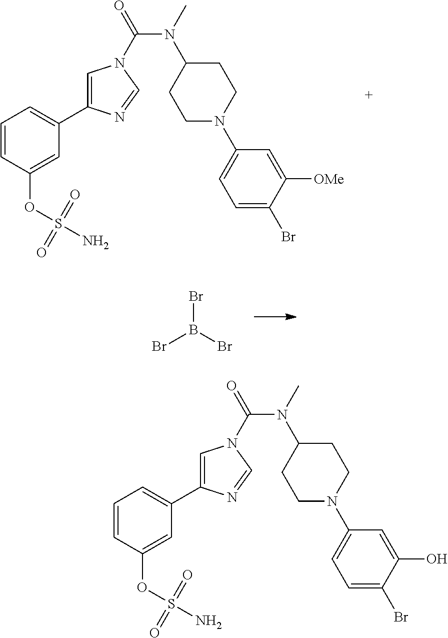

To a suspension of 3-(1-((1-(4-bromo-3-methoxyphenyl)piperidin-4-yl)(methyl)carbamoyl)-1H-imidazol-4-yl)phenyl sulfamate (210 mg, 0.372 mmol) in dry dichloromethane (11 mL) was added dropwise boron tribromide (0.106 mL, 1.117 mmol) at −78° C. The reaction was stirred in the cold for 1 h and then was allowed to stir at room temperature overnight. Once completed, the reaction was quenched with crushed ice. The obtained white solid was purified by column chromatography (dichloromethane/methanol 9:1) to yield a white solid, crystallized from isopropanol. (Yield: 75 mg, 37%).

### Step 7: 3-(1-((1-(4-bromo-3-hydroxyphenyl)piperidin-4-yl)(methyl)carbamoyl)-1H-imidazol-4-yl)phenyl sulfamate hydrochloride

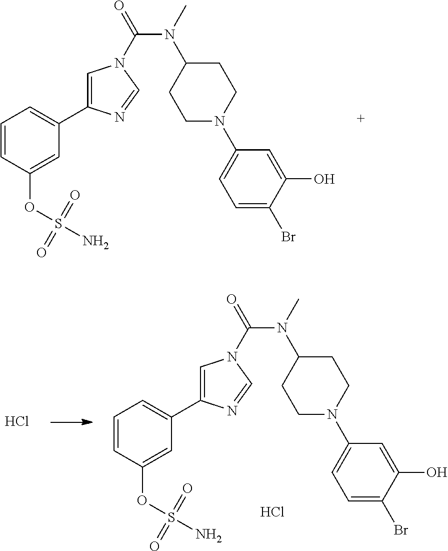 [SMILES: CN(C(=O)n1cnc(-c2cccc(OS(N)(=O)=O)c2)c1)C1CCN(c2ccc(Br)c(O)c2)CC1 ; Cl ; CN(C(=O)n1cnc(-c2cccc(OS(N)(=O)=O)c2)c1)C1CCN(c2ccc(Br)c(O)c2)CC1 ; Cl]

A solution of 3-(1-((1-(4-bromo-3-hydroxyphenyl)piperidin-4-yl)(methyl)carbamoyl)-1H-imidazol-4-yl)phenyl sulfamate (70 mg, 0.127 mmol) in methanol (1 mL) and ethyl acetate (1 mL) was treated with 2M HCl solution in diethyl ether (0.064 mL, 0.127 mmol). The reaction mixture was stirred for 30 min. and filtered. The filter cake was washed with methanol and diethyl ether and dried under vacuum to yield an off-white powder. (Yield: 37 mg, 50%).

¹H NMR (DMSO), δ (ppm): 10.67 (1H, br), 8.52 (1H, s), 8.23 (1H, s), 8.07 (2H, s), 7.83 (1H, md, J=7.8 Hz), 7.79 (1H, t, J=1.4 Hz), 7.51 (1H, t, J=8.0 Hz), 7.51 (1H, br), 7.22 (1H, ddd, J=0.9, 2.4, 8.1 Hz), 7.13 (1H, br), 6.88 (1H, br), 4.29 (1H, br), 3.64 (2H, m), 3.34 (2H, m), 3.0 (3H, s), 2.36 (2H, br), 1.95 (2H, d, J=11.2 Hz).

¹³C NMR (DMSO), δ (ppm): 154.8, 150.7, 150.3, 137.9, 137.8, 133.6, 133.4, 130.2, 123.1, 121.2, 118.7, 115.8, 111.4, 55.2, 52.7, 31.5, 26.

### Example 30

### 3-(1-((1-(benzo[d][1,3]dioxol-5-ylmethyl)piperidin-4-yl)(methyl)carbamoyl)-1H-imidazol-4-yl)phenyl sulfamate hydrochloride

### Step1: N-(1-(benzo[d][1, 3]dioxol-5-ylmethyl)piperidin-4-yl)-4-(3-hydroxyphenyl)-N-methyl-1 H-imidazole-1-carboxamide

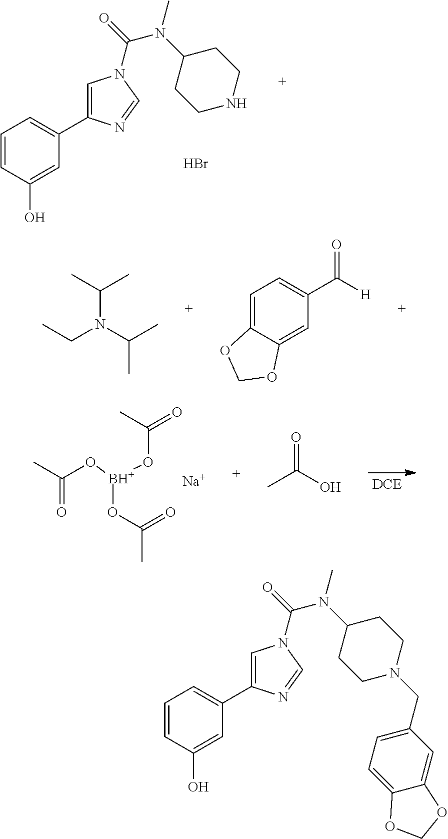

To a stirred suspension of 4-(3-hydroxyphenyl)-N-methyl-N-(piperidin-4-yl)-1H-imidazole-1-carboxamide (Intermediate 13) (250 mg, 0.832 mmol) in 1,2-dichloroethane (7.24 mL) was added N,N-Diisopropylethylamine (0.581 mL, 3.33 mmol), followed by addition of benzo[d][1,3]dioxole-5-carbaldehyde (250 mg, 1.665 mmol) and the reaction was allowed to stir at room temperature for 30 min. Then sodium triacetoxyhydroborate (353 mg, 1.665 mmol) and acetic acid (0.047 mL, 0.583 mmol) were added and the stirring was continued at room temperature. After completion, the reaction was quenched upon addition of crushed ice and the obtained white solid was collected Recrystallization from isopropanol afforded the title product. (Yield: 0.208 g, 55%).

### Step2: 3-(1-((1-(benzo[d][1, 3]dioxol-5-ylmethyl)piperidin-4-yl)(methyl)carbamoyl)-1H-imidazol-4-yl)phenyl sulfamate

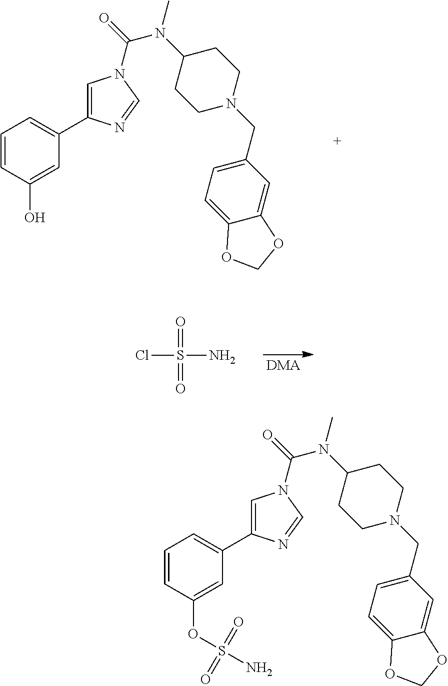

To a turbid solution of N-(1-(benzo[d][1,3]dioxol-5-ylmethyl)piperidin-4-yl)-4-(3-hydroxyphenyl)-N-methyl-1H-imidazole-1-carboxamide (208 mg, 0.479 mmol) in N,N-dimethylacetamide (1.9 mL) was added sulfamoyl chloride (221 mg, 1.915 mmol) and the reaction mixture was stirred at room temperature. After 48 h of stirring the reaction mixture was subjected to column chromatography (silica gel H; gradient dichloromethane/methanol 9:1) to give the title product. (Yield: 102 mg, 37%).

### Step 3: 3-(1-((1-(benzo[d][1, 3]dioxol-5-ylmethyl)piperidin-4-yl)(methyl)carbamoyl)-1H-imidazol-4-yl)phenyl sulfamate hydrochloride

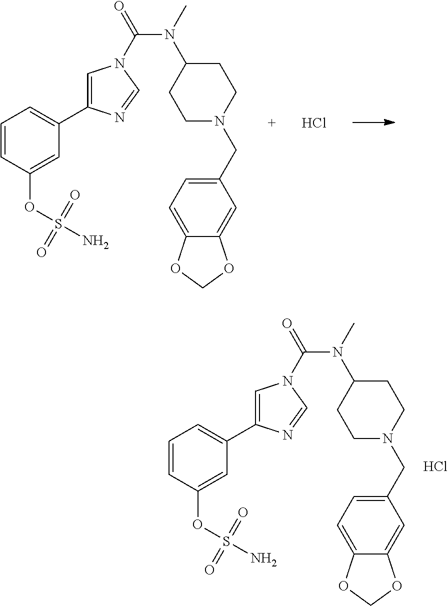 [SMILES: CN(C(=O)n1cnc(-c2cccc(OS(N)(=O)=O)c2)c1)C1CCN(Cc2ccc3c(c2)OCO3)CC1 ; Cl ; CN(C(=O)n1cnc(-c2cccc(OS(N)(=O)=O)c2)c1)C1CCN(Cc2ccc3c(c2)OCO3)CC1 ; Cl]

To a clear cold solution of 3-(1-((1-(benzo[d][1,3]dioxol-5-ylmethyl)piperidin-4-yl)(methyl)carbamoyl)-1H-imidazol-4-yl)phenyl sulfamate (102 mg, 0.199 mmol) in a mixture of ethyl acetate (2.5 mL) and methanol (2.5 mL) was added dropwise HCl solution (2 M in diethyl ether) (0.397 mL, 0.794 mmol). The reaction was allowed to stir in the cold for couple of hours and then solvents were removed under reduced pressure. The residue was recrystallized from a mixture of isopropanol diethyl ether to afford 3-(1-((1-(benzo[d][1,3]dioxol-5-ylmethyl)piperidin-4-yl)(methyl)carbamoyl)-1H-imidazol-4-yl)phenyl sulfamate hydrochloride (Yield: 105 mg, 87%).

¹H NMR (DMSO), δ (ppm): 11.09 (1H, s), 8.51 (1H, s), 8.21 (1H, s), 8.07 (2H, s), 7.82 (1H, d, J=7.9 Hz), 7.78 (1H, t, J=2.0 Hz), 7.50 (1H, t, J=7.9 Hz), 7.28 (1H, s), 7.22 (1H, dd, J=2.4, 8.4 Hz), 7.04 (1H, dd, J=1.0, 8.0 Hz), 6.98 (1H, d, J=8.0 Hz), 6.07 (2H, s), 4.20 (1H, br), 4.16 (2H, d, J=5.0 Hz), 3.38 (2H, md), 3.04 (2H, mq), 2.95 (3H, s), 2.37 (2H, mq), 1.95 (2H, d, J=12.5 Hz).

¹³C NMR (DMSO), δ (ppm): 150.7, 150.4, 148.1, 147.4, 138.1, 137.9, 133.7, 130.2, 125.5, 123.2, 123, 121.1, 118.6, 115.7, 111.3, 108.4, 101.5, 58.5, 52.2, 50, 31.6, 24.8.

### Example 31

### 4-(3-carbamoylphenyl)-N-(1-(4-fluoro-3-methoxybenzyl)piperidin-4-yl)-N-methyl-1H-imidazole-1-carboxamide hydrochloride

### Step1: 4-(3-carbamoylphenyl)-N-(1-(4-fluoro-3-methoxybenzyl)piperidin-4-yl)-N-methyl-1H-imidazole-1-carboxamide

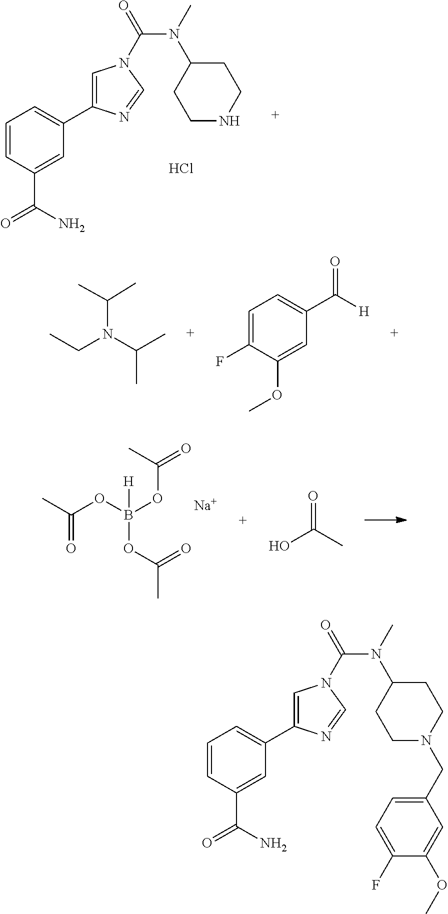

To a suspension of Intermediate 20 (0.4 g, 1.099 mmol) in 1,2-dichloroethane (10 mL) was added N,N-diisopropylethylamine (0.768 mL, 4.40 mmol) followed by addition of 4-fluoro-3-methoxybenzaldehyde (0.339 g, 2.199 mmol). The reaction was allowed to stir under inert atmosphere for 15 min and then sodium triacetoxyhydroborate (0.466 g, 2.199 mmol) and acetic acid (0.065 mL, 1.099 mmol) were added. The reaction mixture was stirred, at room temperature, overnight, then was quenched with water. The phases were separated and the aqueous phase was extracted several times with dichloromethane/isopropanol 7:3. The combined organic layers were dried over MgSO₄, filtered and evaporated. The resultant residue was crystallized from isopropanol to yield an off-white solid. (Yield 258 mg, 50%).

### Step2: 4-(3-carbamoylphenyl)-N-(1-(4-fluoro-3-methoxybenzyl)piperidin-4-yl)-N-methyl-1H-imidazole-1-carboxamide hydrochloride

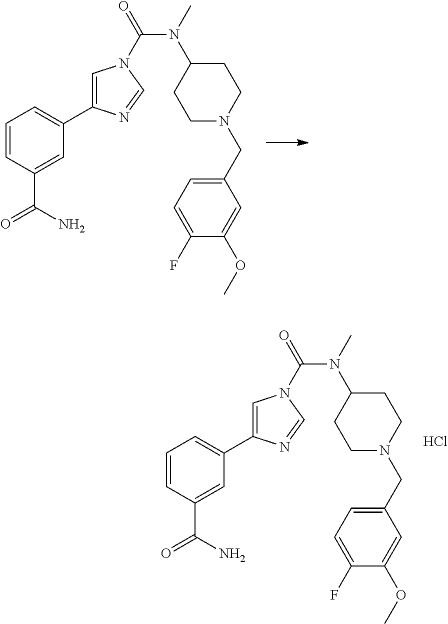 [SMILES: COc1cc(CN2CCC(N(C)C(=O)n3cnc(-c4cccc(C(N)=O)c4)c3)CC2)ccc1F ; COc1cc(CN2CCC(N(C)C(=O)n3cnc(-c4cccc(C(N)=O)c4)c3)CC2)ccc1F ; Cl]

A solution of 4-(3-carbamoylphenyl)-N-(1-(4-fluoro-3-methoxybenzyl)piperidin-4-yl)-N-methyl-1H-imidazole-1-carboxamide (100 mg, 0.215 mmol) in methanol (2 mL) was treated with 2M HCl solution in diethyl ether (0.107 mL, 0.215 mmol). The reaction mixture was stirred for 30 min and then filtered. The filter cake was washed with ethyl ether and dried under vacuum to yield 4-(3-carbamoylphenyl)-N-(1-(4-fluoro-3-methoxybenzyl)piperidin-4-yl)-N-methyl-1H-imidazole-1-carboxamide hydrochloride (100 mg, 0.199 mmol, 93% yield) as a white powder.

¹H NMR (DMSO), δ (ppm): 11.40 (1H, s), 8.72 (1H, s), 8.42 (1H, t, J=1.7 Hz), 8.29 (1H, s), 8.08 (1H, s), 8.03 (1H, td, J=1.3, 8.0 Hz), 7.82 (1H, td, J=1.0, 7.8 Hz), 7.67 (1H, dd, J=1.7, 8.4 Hz), 7.52 (1H, t, J=7.8 Hz), 7.47 (1H, s), 7.28 (1H, dd, J=8.3, 11.5 Hz), 7.11 (1H, m), 4.22 (1H, m), 4.24 (1H, d, J=5.0), 3.89 (3H, s), 3.39 (2H, md), 3.08 (2H, mq, J=11.7 Hz), 2.98 (3H, s), 2.44 (2H, dq, J=3.0, 12.5 Hz), 1.96 (2H, d, J=12.5 Hz).

¹³C NMR (DMSO), δ (ppm): 167.6, 152.8, 151.1, 150.1, 147.1, 147.1, 137.8, 137.6, 134.8, 131, 128.8, 127.7, 126.9, 126.7, 126.6, 124.4, 123.9, 123.9, 116.9, 116, 115.9, 115.7, 58.4, 56.1, 52.2, 50.2, 31.5, 24.7.

### Example 32

### 4-(3-carbamoylphenyl)-N-(1-(2-fluoro-5-methoxybenzyl)piperidin-4-yl)-N-methyl-1H-imidazole-1-carboxamide hydrochloride

### Step1: 4-(3-carbamoylphenyl)-N-(1-(2-fluoro-5-methoxybenzyl)piperidin-4-yl)-N-methyl-1H-imidazole-1-carboxamide

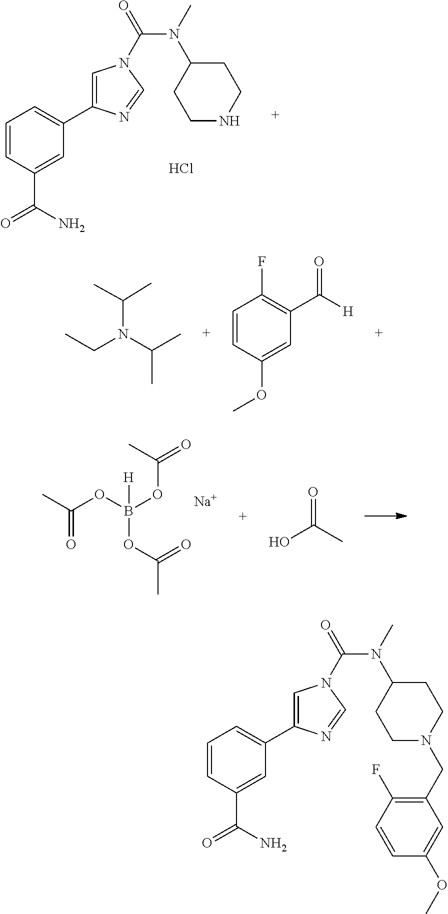

To a suspension of Intermediate 20 (0.3 g, 0.723 mmol) in 1,2-dichloroethane (10 mL) was added N,N-diisopropylethylamine (0.50 mL, 2.89 mmol) followed by addition of 2-fluoro-5-methoxybenzaldehyde (0.339 g, 2.199 mmol). The reaction was allowed to stir under inert atmosphere for 15 min and then sodium triacetoxyhydroborate (0.466 g, 2.199 mmol) and acetic acid (0.065 mL, 1.099 mmol) were added. The reaction mixture was stirred, at room temperature, overnight, then was quenched with water. The phases were separated and the aqueous phase was extracted several times with dichloromethane/isopropanol 7:3. The combined organic layers were dried over MgSO₄, filtered and evaporated. The resultant residue was crystallized from isopropanol to yield an off-white solid. (Yield 224 mg, 44%).

### Step2: 4-(3-carbamoylphenyl)-N-(1-(2-fluoro-5-methoxybenzyl)piperidin-4-yl)-N-methyl-1H-imidazole-1-carboxamide hydrochloride

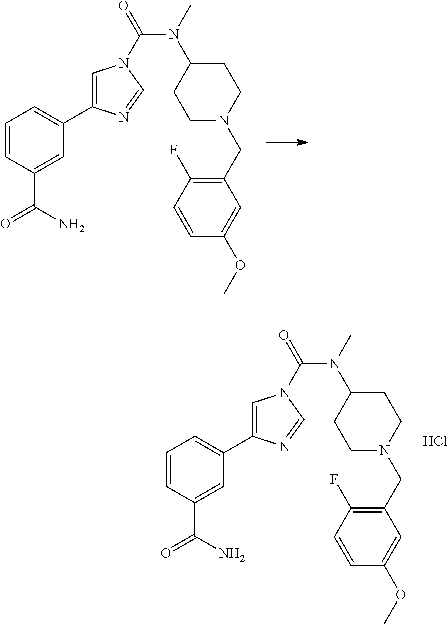 [SMILES: COc1ccc(F)c(CN2CCC(N(C)C(=O)n3cnc(-c4cccc(C(N)=O)c4)c3)CC2)c1 ; COc1ccc(F)c(CN2CCC(N(C)C(=O)n3cnc(-c4cccc(C(N)=O)c4)c3)CC2)c1 ; Cl]

A solution of 4-(3-carbamoylphenyl)-N-(1-(2-fluoro-5-methoxybenzyl)piperidin-4-yl)-N-methyl-1H-imidazole-1-carboxamide (100 mg, 0.215 mmol) in methanol (2 mL) was treated with 2M HCl solution in diethyl ether (0.107 mL, 0.215 mmol). The reaction mixture was stirred for 30 min and then filtered. The filter cake was washed with ethyl ether and dried under vacuum to yield the title compound as a white powder. (Yield: 93 mg, 86%).

¹H NMR (DMSO), δ (ppm): 11.34 (1H, s), 8.70 (1H, s), 8.42 (1H, s), 8.28 (1H, s), 8.08 (1H, s), 8.03 (1H, td, J=1.4, 7.8 Hz), 7.82 (1H, td, J=1.3, 7.7 Hz), 7.52 (1H, t, J=7.6 Hz), 7.50 (1H, m), 7.47 (1H, s), 7.24 (1H, t, J=9.1 Hz), 7.05 (1H, dt, J=3.5, 9.1 Hz), 4.28 (2H, d, J=4.4 Hz), 4.25 (1H, s br), 3.79 (3H, s), 3.44 (2H, d, J=11.0 Hz), 3.20 (2H, q, J=11.3 Hz), 2.97 (3H, s), 2.42 (2H, dq, J=3.0, 12.5 Hz), 1.97 (2H, d, J=12.5 Hz).

¹³C NMR (DMSO), δ (ppm): 167.6, 156.1, 155.4, 154.5, 150.1, 137.8, 137.7, 134.8, 131.1, 128.8, 127.7, 126.9, 124.4, 118, 117.5, 117.4, 117.3, 117.2, 116.5, 116.4, 115.7, 55.8, 52, 51.8, 50.2, 31.7, 24.7.

### Example 33

### 4-(3-carbamoylphenyl)-N-(1-(4-methoxybenzyl)piperidin-4-yl)-N-methyl-1H-imidazole-1-carboxamide hydrochloride

### Step1: 4-(3-carbamoylphenyl)-N-(1-(4-methoxybenzyl)piperidin-4-yl)-N-methyl-1H-imidazole-1-carboxamide

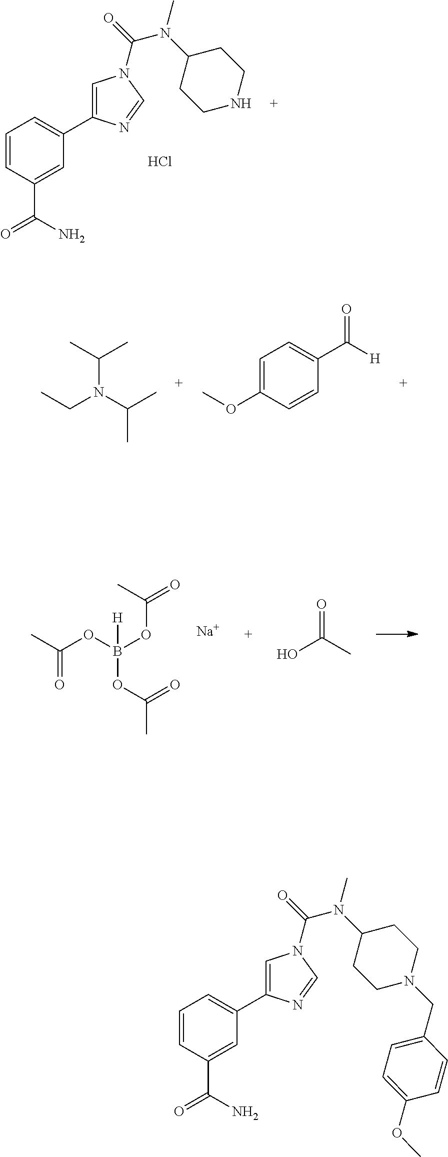

To a suspension of Intermediate 20 (0.3 g, 0.825 mmol) in 1,2-dichloroethane (10 mL) was added N,N-diisopropylethylamine (0.58 mL, 3.30 mmol) followed by addition of 4-methoxybenzaldehyde (0.225 g, 1.649 mmol). The reaction was allowed to stir under inert atmosphere for 15 min and then sodium triacetoxyhydroborate (0.350 g, 1.649 mmol) and acetic acid (0.049 mL, 0.825 mmol) were added. The reaction mixture was stirred, at room temperature, overnight, then was quenched with water. The phases were separated and the aqueous phase was extracted several times with dichloromethane/isopropanol 7:3. The combined organic layers were dried over MgSO₄, filtered and evaporated. The resultant residue was crystallized from isopropanol to yield an off-white solid. (Yield 164 mg, 44%).

### Step2: 4-(3-carbamoylphenyl)-N-(1-(4-methoxybenzyl)piperidin-4-yl)-N-methyl-1H-imidazole-1-carboxamide hydrochloride

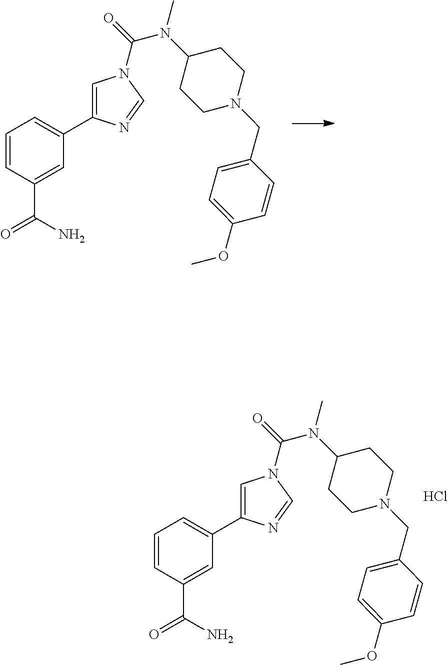 [SMILES: COc1ccc(CN2CCC(N(C)C(=O)n3cnc(-c4cccc(C(N)=O)c4)c3)CC2)cc1 ; COc1ccc(CN2CCC(N(C)C(=O)n3cnc(-c4cccc(C(N)=O)c4)c3)CC2)cc1 ; Cl]

A solution of 4-(3-carbamoylphenyl)-N-(1-(4-methoxybenzyl)piperidin-4-yl)-N-methyl-1H-imidazole-1-carboxamide (100 mg, 0.223 mmol) in methanol (2 mL) was treated with 2M HCl solution in diethyl ether (0.134 mL, 0.268 mmol). The reaction mixture was stirred for 30 min and then filtered. The filter cake was washed with ethyl ether and dried under vacuum to yield the title compound as a white powder. (Yield: 95 mg, 88%).

¹H NMR (DMSO), δ (ppm): 10.53 (1H, s), 8.36 (1H, t, J=1.7 Hz), 8.35 (1H, s), 8.15 (1H, s), 8.04 (1H, s), 7.99 (1H, ddd, J=1.2, 1.6, 7.8 Hz), 7.78 (1H, ddd, J=1.1, 1.6, 7.7 Hz), 7.50 (2H, md, J=8.7 Hz), 7.49 (1H, t, J=7.7 Hz), 7.43 (1H, s), 7.02 (2H, md, J=8.7 Hz), 4.19 (2H, d, J=5.0 Hz), 4.16 (1H, s br), 3.78 (3H, s), 3.39 (2H, d, J=13.0 Hz), 3.06 (2H, q, J=11.5 Hz), 2.95 (3H, s), 2.28 (2H, dq, J=3.3, 12.8 Hz), 1.97 (2H, d, J=13.0 Hz).

¹³C NMR (DMSO), δ (ppm): 167.6, 160, 150.1, 137.8, 137.7, 134.8, 132.9, 131, 128.8, 127.7, 126.9, 124.4, 121.6, 115.6, 114.1, 58.2, 55.2, 49.9, 31.6, 24.7.

### Example 34

### 4-(3-carbamoylphenyl)-N-(1-(2-methoxyphenyl)piperidin-4-yl)-N-methyl-1 H-imidazole-1-carboxamide hydrochloride

### Step1: 1-(2-methoxyphenyl)piperidin-4-one

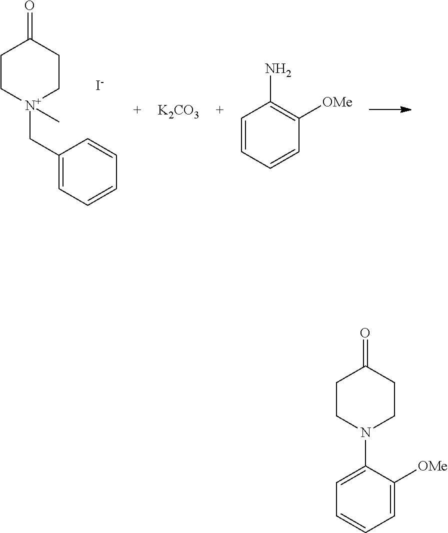

In a 250 mL round-bottomed flask 2-methoxyaniline (3 g, 24.36 mmol), potassium carbonate (0.471 g, 3.41 mmol) were placed in ethanol (80 mL). The mixture was heated at reflux and a suspension of 1-benzyl-1-methyl-4-oxopiperidinium iodide (12.10 g, 36.5 mmol) in water (65 mL) was added over a period of 1 h. The reaction mixture was stirred at reflux for 3 h. Then, it was quenched with water (50 mL) and the solution was extracted with dichloromethane. The organic phase was dried over MgSO₄, filtered and evaporated. The crude mixture was purified by column chromatography (petroleum ether/ethyl acetate, 4:1) and the obtained product was recrystallized from petroleum ether. (Yield: 4.39 g, 88%).

### Step2: 1-(2-methoxyphenyl)-N-methylpiperidin-4-amine

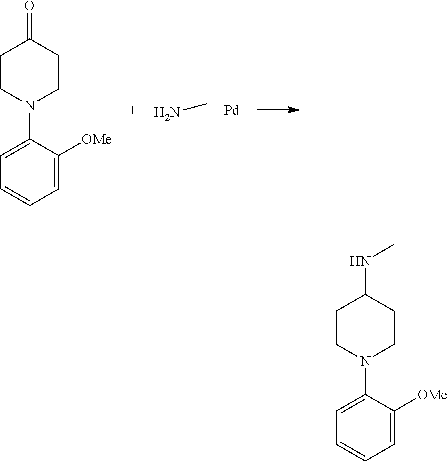

In a 250 mL glass flask was placed methylamine (8.08 mL, 94 mmol) under inert atmosphere. Palladium (10% on charcoal, 0.363 g, 0.341 mmol) was added followed by a solution of 1-(2-methoxyphenyl)piperidin-4-one (4.38 g, 21.34 mmol) in methanol (15 mL). The reaction flask was placed in an autoclave and was charged with 20 atm of hydrogen. The autoclave was heated at 50° C. and stirred for 2 h. The reaction mixture was filtered through celite and the solvent was removed to obtain a light beige solid. (Yield: 2.598 g, 55%).

### Step3: (1-(2-methoxyphenyl)piperidin-4-yl)(methyl)carbamic chloride

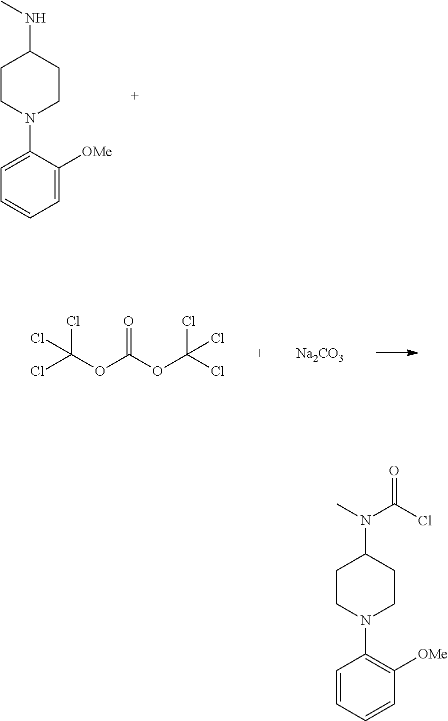

A cooled solution (0° C.) of bis(trichloromethyl)carbonate (1.395 g, 4.70 mmol) in dichloromethane (20 mL) was treated with a solution of 1-(2-methoxyphenyl)-N-methylpiperidin-4-amine (2.59 g, 11.76 mmol) in dichloromethane (20 mL). Then, sodium carbonate (2.492 g, 23.51 mmol) was added portionwise. The reaction mixture was allowed to warm up to room temperature and stirred overnight. Then, it was quenched with water, the organic phase was dried over MgSO₄ and the solvent was removed under reduced pressure. The obtained solid was triturated with petroleum ether, filtered and dried. (Yield: 2.67 g, 80%).

### Step 4: 4-(3-carbamoylphenyl)-N-(1-(2-methoxyphenyl)piperidin-4-yl)-N-methyl-1H-imidazole-1-carboxamide

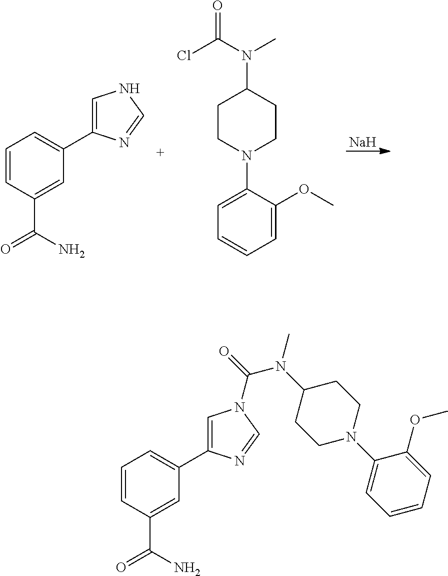

A cold solution of Intermediate 19 (400 mg, 2.137 mmol) in DMF (13.35 mL) was treated with sodium hydride (60% in oil dispersion) (103 mg, 2.56 mmol) and stirred for 30 min. Then, (1-(2-methoxyphenyl)piperidin-4-yl)(methyl)carbamic chloride (604 mg, 2.137 mmol) was added in one portion and the reaction was allowed to heat to room temperature and stirred overnight. Then, the reaction was transferred to a separatory funnel, extracted with a mixture of dichloromethane and isopropanol, then washed with aqueous 1N HCl and water, respectively. The organic phase was dried over MgSO₄, concentrated and the residue was crystallized from hot isopropanol to yield an off-white solid. (Yield: 0.542 g, 58%).

### Step 5: 4-(3-carbamoylphenyl)-N-(1-(2-methoxyphenyl)piperidin-4-yl)-N-methyl-1H-imidazole-1-carboxamide hydrochloride

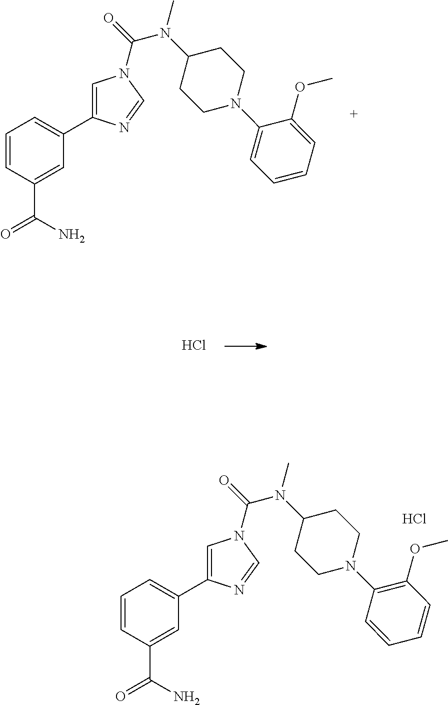 [SMILES: COc1ccccc1N1CCC(N(C)C(=O)n2cnc(-c3cccc(C(N)=O)c3)c2)CC1 ; Cl ; COc1ccccc1N1CCC(N(C)C(=O)n2cnc(-c3cccc(C(N)=O)c3)c2)CC1 ; Cl]

A solution of 4-(3-carbamoylphenyl)-N-(1-(2-methoxyphenyl)piperidin-4-yl)-N-methyl-1H-imidazole-1-carboxamide (100 mg, 0.231 mmol) in a mixture of methanol (1 mL) and ethyl acetate (1 mL) was treated with 2M HCl solution in diethyl ether (0.138 mL, 0.277 mmol). The reaction mixture was stirred for 30 min and then filtered. The filter cake was washed with diethyl ether and dried under vacuum to yield the title product as an off-white solid. (Yield: 0.110 g, quantitative).

¹H NMR (DMSO), δ (ppm): 12.11 (1H, br), 8.81 (1H, s), 8.46 (1H, s), 8.37 (1H, s), 8.11 (1H, s), 8.06 (1H, td, J=1.3, 7.8 Hz), 7.84 (1H, br), 7.84 (1H, dt, J=1.3, 7.8 Hz), 7.54 (1H, t, J=7.8 Hz), 7.48 (1H, s), 7.46 (1H, br), 7.29 (1H, d, J=7.9 Hz), 7.11 (1H, t, J=7.5 Hz), 4.39 (1H, s br), 3.95 (3H, s), 3.82 (2H, br), 3.63 (2H, d, J=11.1 Hz), 3.07 (3H, s), 2.66 (2H, br), 2.03 (2H, d, J=11.8 Hz).

¹³C NMR (DMSO), δ (ppm): 167.6, 151.9, 150.1, 137.8, 137.5, 134.8, 130.8, 128.9, 127.7, 127, 124.4, 122, 121.2, 115.8, 113.7, 56.4, 52.6, 52, 31.8, 25.4.

### Example 35

### 4-(3-carbamoylphenyl)-N-(1-(4-hydroxyphenyl)piperidin-4-yl)-N-methyl-1H-imidazole-1-carboxamide hydrochloride

### Step1: 4-(3-carbamoylphenyl)-N-(1-(4-methoxyphenyl)piperidin-4-yl)-N-methyl-1H-imidazole-1-carboxamide

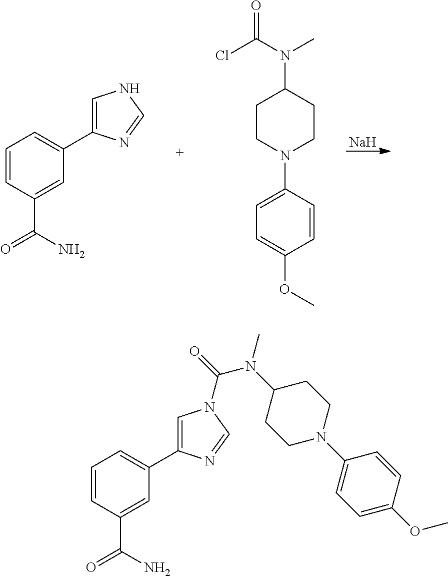

A cold solution of Intermediate 19 (500 mg, 2.67 mmol) in DMF (15 mL) was treated with sodium hydride (60% in oil dispersion) (121 mg, 3.28 mmol) and stirred for 30 min. Then, Intermediate 6 (906 mg, 3.21 mmol) was added in one portion and the reaction was allowed to heat to room temperature and stirred overnight. Then, the reaction was transferred to a separatory funnel, extracted with a mixture of dichloromethane and isopropanol, and then washed with aqueous 1N HCl and water, respectively. The organic phase was dried over MgSO₄, concentrated and the residue was chromatographed (dichloromethane/methanol 10:1) followed by trituration with diethyl ether to yield an off-white solid. (Yield: 0.148 g, 13%).

### Step2: 4-(3-carbamoylphenyl)-N-(1-(4-hydroxyphenyl)piperidin-4-yl)-N-methyl-1H-imidazole-1-carboxamide

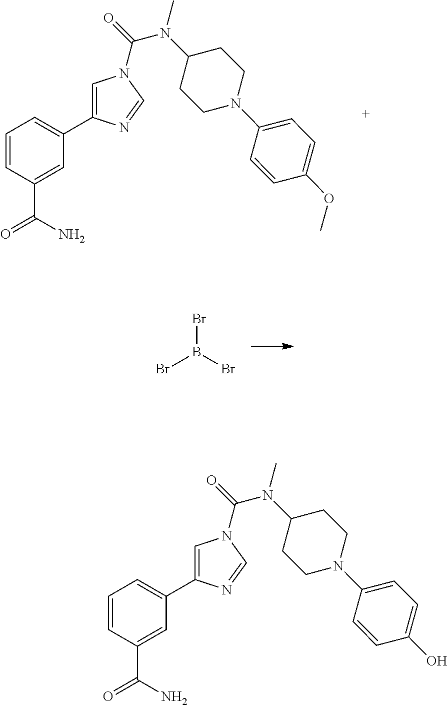

To a suspension of 4-(3-carbamoylphenyl)-N-(1-(4-methoxyphenyl)piperidin-4-yl)-N-methyl-1H-imidazole-1-carboxamide (140 mg, 0.323 mmol) in dry dichloromethane (9.23 mL) at −78° C. was added boron tribromide (0.092 mL, 0.969 mmol). The reaction was allowed to stir in the cold for 15 min and then at room temperature overnight. Then, the reaction was quenched with crushed ice, neutralized with saturated aqueous solution of NaHCO₃ and stirred for 1 h. The resultant precipitate was filtered off, washed with water, and dried under vacuum to yield an off-white solid. (Yield: 89 mg, 66%).

### Step3: 4-(3-carbamoylphenyl)-N-(1-(4-hydroxyphenyl)piperidin-4-yl)-N-methyl-1H-imidazole-1-carboxamide hydrochloride

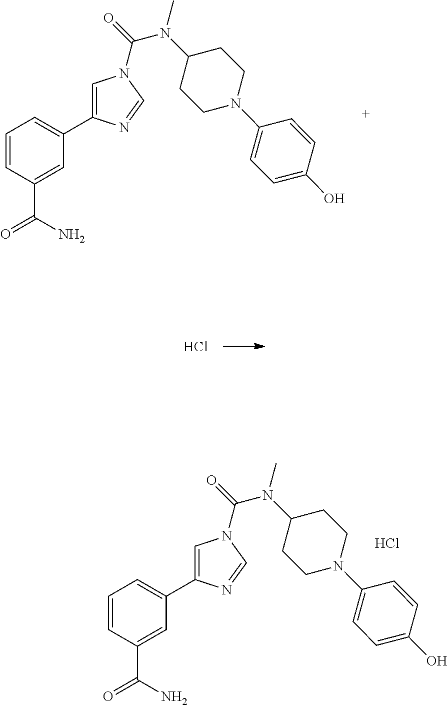 [SMILES: CN(C(=O)n1cnc(-c2cccc(C(N)=O)c2)c1)C1CCN(c2ccc(O)cc2)CC1 ; Cl ; CN(C(=O)n1cnc(-c2cccc(C(N)=O)c2)c1)C1CCN(c2ccc(O)cc2)CC1 ; Cl]

A solution of 4-(3-carbamoylphenyl)-N-(1-(4-hydroxyphenyl)piperidin-4-yl)-N-methyl-1H-imidazole-1-carboxamide (89 mg, 0212 mmol) in a mixture of methanol (1 mL) and ethyl acetate (1 mL) was treated with 2M HCl solution in diethyl ether (0.106 mL, 0.212 mmol). The reaction mixture was stirred for 30 min and then filtered. The filter cake was washed with methanol and diethyl ether then dried under vacuum to yield the title product as an off-white solid. (Yield: 0.081 g, 84%).

¹H NMR (DMSO), δ (ppm): 12.94 (1H, s), 10.12 (1H, s br), 8.72 (1H, s br), 8.42 (1H, s), 8.31 (1H, s), 8.08 (1H, s), 8.04 (1H, md, J=7.9 Hz), 7.83 (1H, md, J=7.9 Hz), 7.71 (2H, md, J=8.7 Hz), 7.53 (1H, t, J=7.7 Hz), 7.47 (1H, s), 6.91 (2H, md, J=8.7 Hz), 4.47 (1H, s br), 3.54 (2H, d, J=11.2 Hz), 3.04 (3H, s), 2.71 (2H, dq, J=12.5 Hz), 2.04 (2H, d, J=12.3 Hz).

¹³C NMR (DMSO), δ (ppm): 167.6, 158.2, 150.3, 137.9, 137.9, 137.9, 134.8, 133.8, 131.2, 128.8, 127.7, 126.9, 124.4, 122.7, 116.2, 115.7, 54.7, 51.5, 31.7, 25.3.

### Example 36

### 4-(3-carbamoylphenyl)-N-(1-(2-hydroxybenzyl)piperidin-4-yl)-N-methyl-1 H-imidazole-1-carboxamide hydrochloride

### Step1: 4-(3-carbamoylphenyl)-N-(1-(2-hydroxybenzyl)piperidin-4-yl)-N-methyl-1H-imidazole-1-carboxamide

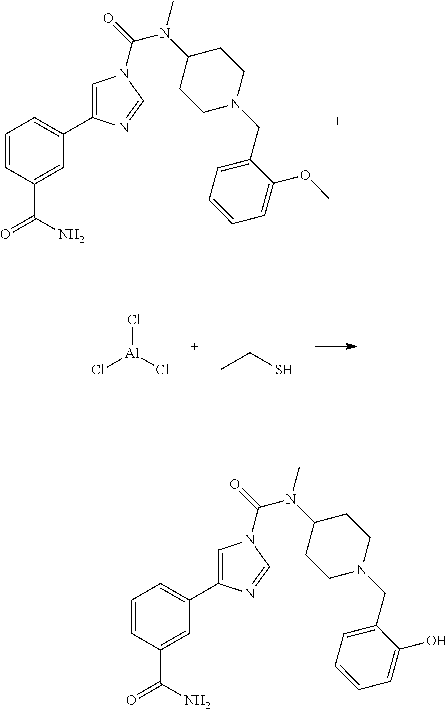

To a −78° C. cold suspension of 4-(3-carbamoylphenyl)-N-(1-(2-methoxybenzyl)piperidin-4-yl)-N-methyl-1H-imidazole-1-carboxamide (180 mg, 0.402 mmol) (Intermediate 21) in dry DCM (6 mL) was added aluminum trichloride (54 mg, 0.402 mmol) followed by addition of ethanethiol (25 mg, 0.402 mmol). The reaction was allowed to stir in the cold for 15 minutes and then at room temperature overnight. Then, the reaction was quenched with crushed ice, neutralized with saturated aqueous solution of NaHCO₃ and stirred for 1 h. The resultant precipitate was filtered, washed with water, and purified by column chromatography (dichloromethane/methanol 9:1). Crystallization from isopropanol afforded the title product as an off-white solid. (Yield: 128 mg, 73%).

### Step2: 4-(3-carbamoylphenyl)-N-(1-(2-hydroxybenzyl)piperidin-4-yl)-N-methyl-1H-imidazole-1-carboxamide hydrochloride

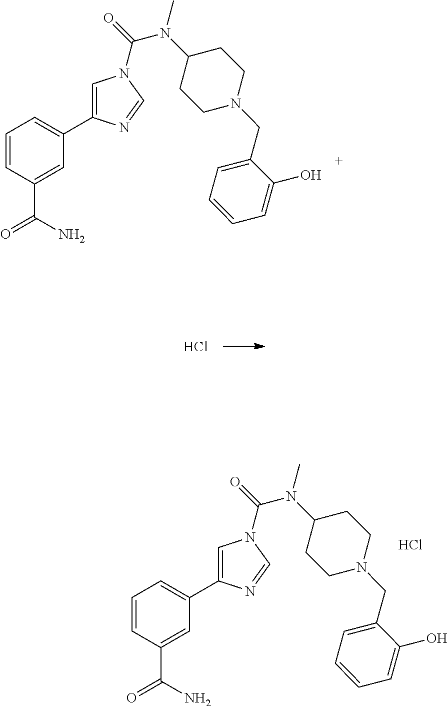 [SMILES: CN(C(=O)n1cnc(-c2cccc(C(N)=O)c2)c1)C1CCN(Cc2ccccc2O)CC1 ; Cl ; CN(C(=O)n1cnc(-c2cccc(C(N)=O)c2)c1)C1CCN(Cc2ccccc2O)CC1 ; Cl]

A solution of 4-(3-carbamoylphenyl)-N-(1-(2-hydroxybenzyl)piperidin-4-yl)-N-methyl-1H-imidazole-1-carboxamide (100 mg, 0.231 mmol) in methanol (2 mL) was treated with 2M HCl solution in diethyl ether (0.14 mL, 0.277 mmol). The reaction mixture was stirred for 30 min and then filtered. The filter cake was washed with diethyl ether then dried under vacuum to yield the title product as an off-white solid. (Yield: 103 mg, 95%).

¹H NMR (DMSO), δ (ppm): 10.45 (1H, s br), 10.27 (1H, s), 8.63 (1H, s), 8.41 (1H, t, J=1.5 Hz), 8.26 (1H, s), 8.07 (1H, s), 8.02 (1H, ddd, J=1.1, 1.7, 7.7 Hz), 7.81 (1H, ddd, J=1.1, 1.6, 7.7 Hz), 7.55-7.49 (2H, m), 7.46 (1H, s), 7.27 (1H, m), 6.99 (1H, dd, J=1.0, 8.1 Hz), 6.87 (1H, dt, J=1.1, 7.5 Hz), 4.23 (1H, m br), 4.18 (2H, d, J=4.8 Hz), 3.44 (2H, d, J=11.5 Hz), 3.16 (2H, m), 2.96 (3H, s), 2.34 (2H, dq, J=3.5, 12.5 Hz), 1.95 (2H, d, J=12.3 Hz),

¹³C NMR (DMSO), (ppm): 167.6, 156.7, 150.3, 138.1, 137.8, 134.8, 133.4, 131.4, 131.1, 128.8, 127.6, 126.8, 124.3, 119.2, 115.9, 115.7, 115.6, 53.5, 52.1, 50.3, 31.7, 24.8.

### Example 37

### 4-(3-carbamoylphenyl)-N-(1-(2-hydroxyphenyl)piperidin-4-yl)-N-methyl-1H-imidazole-1-carboxamide hydrochloride

### Step1: 4-(3-carbamoylphenyl)-N-(1-(2-hydroxyphenyl)piperidin-4-yl)-N-methyl-1H-imidazole-1-carboxamide

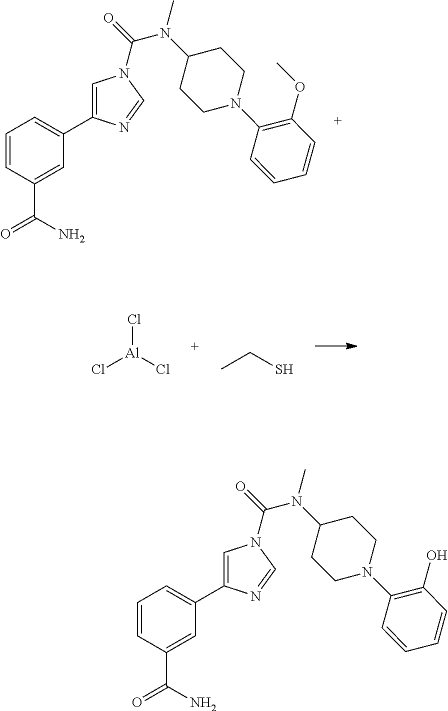

To a −78° C. cold suspension of 4-(3-carbamoylphenyl)-N-(1-(2-methoxyphenyl)piperidin-4-yl)-N-methyl-1H-imidazole-1-carboxamide (150 mg, 0.346 mmol) (Step 4 in Example 34) in dry dichloromethane (3 mL) added aluminum trichloride (231 mg, 1.730 mmol) followed by ethanethiol (0.05 mL, 0.692 mmol). The reaction was allowed to stir in the cold for 15 minutes and then at room temperature overnight. Then, the reaction was quenched with crushed ice, neutralized with saturated aqueous solution of NaHCO₃ and stirred for 1 h. The resultant precipitate was filtered, washed with water, and purified by column chromatography (dichloromethane/methanol 9:1). Crystallization from isopropanol afforded the title compound as an off-white solid. (Yield: 77 mg, 53%).

### Step2: 4-(3-carbamoylphenyl)-N-(1-(2-hydroxyphenyl)piperidin-4-yl)-N-methyl-1H-imidazole-1-carboxamide hydrochloride

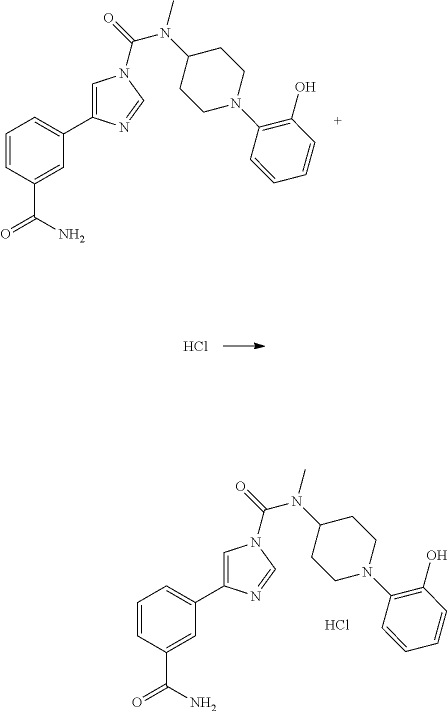 [SMILES: CN(C(=O)n1cnc(-c2cccc(C(N)=O)c2)c1)C1CCN(c2ccccc2O)CC1 ; Cl ; CN(C(=O)n1cnc(-c2cccc(C(N)=O)c2)c1)C1CCN(c2ccccc2O)CC1 ; Cl]

A solution of 4-(3-carbamoylphenyl)-N-(1-(2-hydroxyphenyl)piperidin-4-yl)-N-methyl-1H-imidazole-1-carboxamide (77 mg, 0.184 mmol) in methanol (1 mL) was treated with 2M HCl solution in diethyl ether (0.11 mL, 0.220 mmol). The reaction mixture was stirred for 30 min and then filtered. The filter cake was washed with diethyl ether then dried under vacuum to yield the title product as a white solid. (Yield: 82 mg, 99%).

¹H NMR (DMSO), δ (ppm): 11.64 (1H, br), 11.39 (1H, s br), 8.59 (1H, s), 8.42 (1H, t, J=1.5 Hz), 8.27 (1H, s), 8.08 (1H, s), 8.03 (1H, ddd, J=1.0, 1.5, 7.6 Hz), 7.82 (1H, ddd, J=1.0, 1.5, 7.7 Hz), 7.77 (1H, s br), 7.52 (1H, t, J=7.5 Hz), 7.46 (1H, s), 7.33 (1H, t, J=7.5 Hz), 7.15 (1H, d, J=8.1 Hz), 6.97 (1H, t, J=7.8 Hz), 4.40 (1H, s br), 3.89 (2H, m), 3.64 (2H, m), 3.05 (3H, s), 2.62 (2H, m), 2.06 (2H, d, J=12.4 Hz).

¹³C NMR (DMSO), δ (ppm): 167.7, 150.5, 150.2, 138.5, 137.9, 134.8, 131.8, 130.6, 128.8, 127.6, 126.7, 124.3, 122.1, 119.8, 117.4, 115.5, 52.7, 51.9, 31.8, 25.4.

### Example 38

### 4-(3-carbamoylphenyl)-N-(1-(2-methoxybenzyl)piperidin-4-yl)-N-methyl-1H-imidazole-1-carboxamide hydrochloride

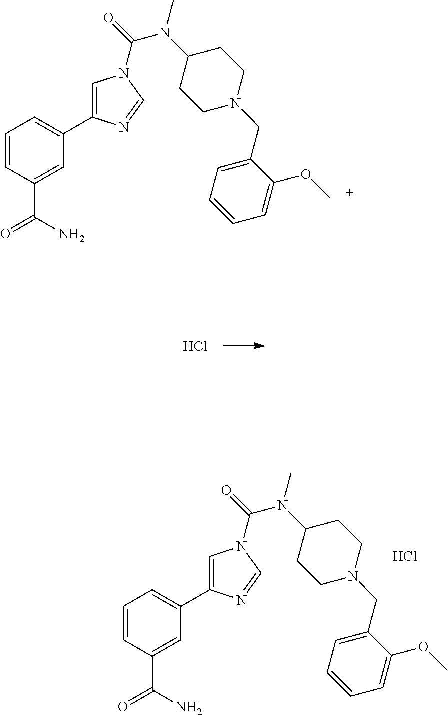 [SMILES: COc1ccccc1CN1CCC(N(C)C(=O)n2cnc(-c3cccc(C(N)=O)c3)c2)CC1 ; Cl ; COc1ccccc1CN1CCC(N(C)C(=O)n2cnc(-c3cccc(C(N)=O)c3)c2)CC1 ; Cl]

A solution of 4-(3-carbamoylphenyl)-N-(1-(2-methoxybenzyl)piperidin-4-yl)-N-methyl-1H-imidazole-1-carboxamide (100 mg, 0.223 mmol) (Intermediate 21) in methanol (2 mL) was treated with 2M HCl solution in diethyl ether (0.13 mL, 0.268 mmol). The reaction mixture was stirred for 30 min and then filtered. The filter cake was washed with diethyl ether then dried under vacuum to yield the title product as an off-white solid. (Yield: 113 mg, 94%).

¹H NMR (DMSO), δ (ppm): 10.71 (1H, s), 8.65 (1H, s br), 8.42 (1H, s), 8.27 (1H, s), 8.08 (1H, s br), 8.03 (1H, td, J=1.2, 7.8 Hz), 7.82 (1H, td, J=1.1, 7.8 Hz), 7.62 (1H, dd, J=1.5, 7.5 Hz), 7.52 (1H, t, J=7.7 Hz), 7.49-7.42 (2H, m), 7.12 (1H, d, J=8.5 Hz), 7.03 (1H, dt, J=0.8, 7.5 Hz), 4.22 (1H, s br), 4.21 (2H, d, J=4.8 Hz), 3.85 (3H, s), 3.42 (2H, d, J=11.5 Hz), 3.14 (2H, m), 2.96 (3H, s), 2.39 (2H, dq, J=3.0, 12.5 Hz), 1.94 (2H, d, J=12.0 Hz).

¹³C NMR (DMSO), δ (ppm): 167.6, 158.1, 150.2, 138, 137.8, 134.8, 133.4, 131.5, 131.3, 128.8, 127.6, 126.8, 124.3, 120.5, 117.5, 115.6, 111.5, 55.8, 53.2, 52.1, 50.4, 31.6, 24.8.

### Example 39

### 4-(4-carbamoylphenyl)-N-(1-(3-methoxybenzyl)piperidin-4-yl)-N-methyl-1H-imidazole-1-carboxamide hydrochloride

### Step1: 4-(1H-imidazol-4-yl)benzonitrile

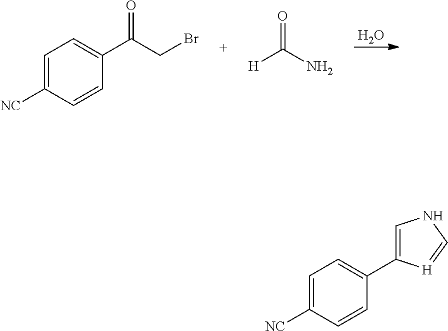

A suspension of 4-(2-bromoacetyl)benzonitrile (4.3 g, 19.19 mmol) in formamide (9.53 mL, 240 mmol) was treated with water (0.9 mL, 19.19 mmol) and the resulting mixture was heated at 140° C. under vigorous stirring for 3 hours. Then, the reaction mixture was cooled to room temperature and diluted with 40 mL of water. The suspension obtained was acidified with 2N HCl until pH 3 and the precipitate formed was filtered off. The mother liqueur was basified with 1N NaOH solution until pH 12 and the suspension formed was cooled to 0° C. The precipitate was filtered recrystallized from ethyl acetate to yield a pale yellow solid. (Yield: 2.234 g, 69%)

### Step2: 4-(1H-imidazol-4-yl)benzamide

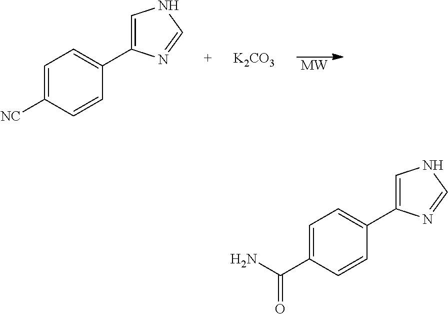

A mixture of 4-(1H-imidazol-4-yl)benzonitrile (2 g, 11.82 mmol) and potassium carbonate (0.327 g, 2.364 mmol) in water (10 mL) was heated in the microwave oven (P=100 W; T=150° C.) for 1 h 30 m. Then, the reaction mixture was diluted with 40 mL of water and extracted with dichloromethane/isopropanol 7:3. The organics were dried over MgSO₄ and the solvents removed under vacuum. The resulting crude material was purified by column chromatography and the product was precipitated from petroleum ether to yield a pale orange solid. (Yield: 1.01 g, 46%).

### Step3: tert-butyl 4-(4-(4-carbamoylphenyl)-N-methyl-1H-imidazole-1-carboxamido)piperidine-1-carboxylate

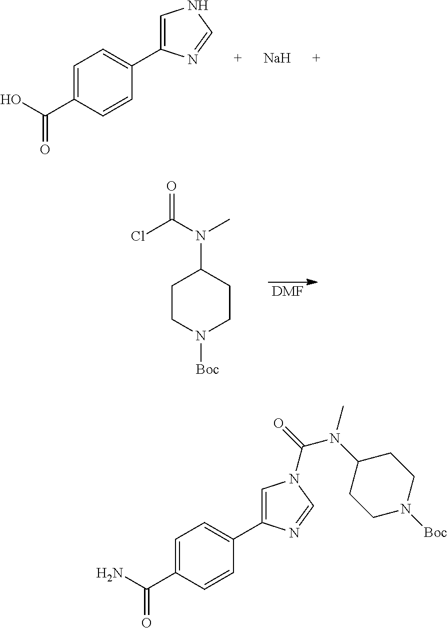

In a 100 mL round-bottomed flask, 4-(1H-imidazol-4-yl)benzamide (1 g, 5.34 mmol) and N,N-dimethylformamide (27 mL) were placed under inert atmosphere. The mixture was cooled to 0° C. and sodium hydride (0.256 g, 6.41 mmol) was added portionwise. Then, the reaction mixture was allowed to warm to room temperature and was stirred for 30 minutes, tert-butyl 4-(chlorocarbonyl(methyl)amino)piperidine-1-carboxylate (1.774 g, 6.41 mmol) was added portionwise and the reaction mixture was stirred for 2.5 hours. The mixture was cooled to 0° C. and quenched with water. The phases were separated. The aqueous phase was extracted several times with dichloromethane/isopropanol 7:3. The combined organic layers were dried over MgSO₄ and concentrated. The resulting residue was purified by column chromatography (dichloromethane/methanol 9:1) to yield a colourless oil that was triturated with ethyl ether. The precipitate was filtered and dried under vacuum. (Yield: 1.59 g, 70%).

### Step4: 4-(3-carbamoylphenyl)-N-methyl-N-(piperidin-4-yl)-1H-imidazole-1-carboxamide hydrochloride

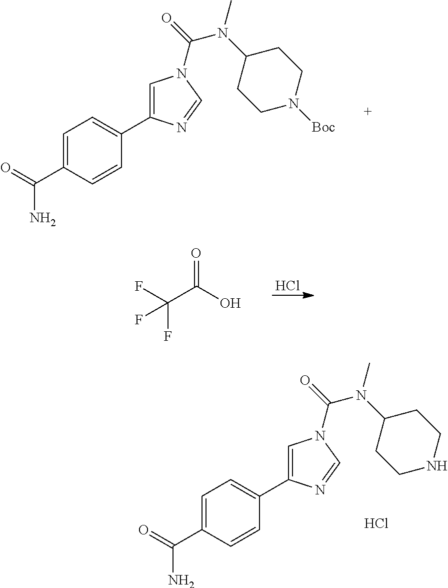 [SMILES: CN(C(=O)n1cnc(-c2ccc(C(N)=O)cc2)c1)C1CCN(C(=O)OC(C)(C)C)CC1 ; O=C(O)C(F)(F)F ; Cl ; CN(C(=O)n1cnc(-c2ccc(C(N)=O)cc2)c1)C1CCNCC1 ; Cl]

tert-butyl 4-(4-(4-carbamoylphenyl)-N-methyl-1H-imidazole-1-carboxamido) piperidine-1-carboxylate (1.59 g, 3.72 mmol) was dissolved in trifluoroacetic acid (8 mL), at 0° C., and the reaction was allowed to heat to room temperature. The reaction was vigorously stirred at room temperature for 1 hour. Trifluoroacetic acid was removed under reduced pressure and the residue obtained was dissolved in 5 mL of ethyl acetate. The solution was cooled to 0° C. and treated with 2N hydrogen chloride solution in diethyl ether (1.86 mL, 3.72 mmol). The precipitate was filtered, washed with diethyl ether and dried under vacuum. (Yield: 1.174 g, 87%).

### Step5: 4-(4-carbamoylphenyl)-N-(1-(3-methoxybenzyl)piperidin-4-yl)-N-methyl-1H-imidazole-1-carboxamide hydrochloride

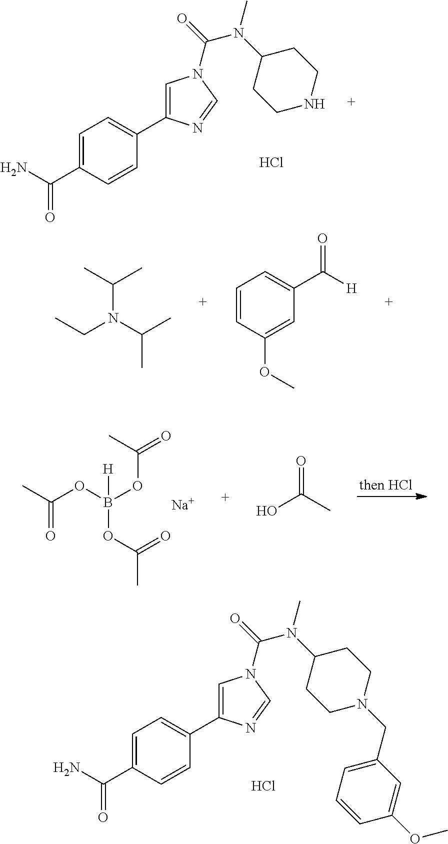

A suspension of 4-(4-carbamoylphenyl)-N-methyl-N-(piperidin-4-yl)-1H-imidazole-1-carboxamide hydrochloride (500 mg, 1.374 mmol) in 1,2-Dichloroethane (19.6 mL) was treated with N,N-diisopropylethylamine (0.960 mL, 5.50 mmol). Then 3-methoxybenzaldehyde (0.168 mL, 1.374 mmol) was added and the reaction was allowed to stir at room temperature. After 30 minutes, sodium triacetoxyhydroborate (583 mg, 2.75 mmol) and acetic acid (0.079 mL, 1.374 mmol) were added and the reaction was stirred overnight. The reaction was quenched with ice and the heterogeneous mixture was transferred to a separating funnel. The aqueous layer was extracted with dichloromethane/isopropanol 7/3. The organic layers were dried over MgSO₄, filtered and evaporated under vacuum. The crude mixture was purified by column chromatography (dichloromethane/isopropanol 9:1) to yield an off-white solid. The solid was dissolved in methanol (1 mL) and was treated with 2N HCl in diethyl ether (0.05 mL). The resulting precipitate was filtered, washed with diethyl ether and dried under vacuum to yield a white solid. (Yield: 33 mg, 5%).

¹H NMR (DMSO), δ (ppm): 10.73 (1H, s), 8.17 (1H, s), 8.13 (1H, s), 7.97 (1H, s), 7.95-7.87 (4H, m), 7.37 (1H, t, J=8.0 Hz), 7.34 (1H, s), 7.27 (1H, s), 7.11 (1H, d, J=7.1 Hz), 7.02 (1H, d, J=7.9 Hz), 4.23 (2H, s br), 4.18 (1H, s br), 3.79 (3H, s), 3.38 (2H, m), 3.09 (2H, m), 2.95 (3H, s), 2.32 (2H, q, J=12.0 Hz), 1.96 (2H, d, J=12.5 Hz).

¹³C NMR (DMSO), δ (ppm): 167.6, 159.4, 151, 140, 138, 136, 132.6, 131.2, 129.9, 127.9, 124.3, 123.3, 116.7, 115.6, 115.1, 58.8, 55.2, 52.2, 50.4, 31.5, 24.9.

### 2. Biological Efficacy of Compounds of the Invention

In vitro/ex vivo Protocol for FAAH Activity Determination

To evaluate the selectivity of test compounds in inhibiting FAAH activity, male NMRI mice were administrated following instillation with 1 mg/kg compound and were sacrificed after 8 h treatment. Liver and brain fragments were removed and processed for enzymatic activity determination.

FAAH activity was measured as the amount of ³H-ethanolamine formed, by liquid scintillation counting, from the hydrolysis of the substrate anandamide (AEA, labeled with ³H on the ethanolamine part of the molecule). The percentage of remaining enzymatic activity was calculated in respect to controls and after blank subtraction. Therefore, a low value for the test compounds indicates a strong inhibitor. A value of 100 indicates that no measurable inhibition took place.

### 1. Animal Treatment

Male NMRI mice (body weight range: 25-35 g) were obtained from Harlan Laboratories (Barcelona, Spain). Animals were kept 10 per cage, under controlled environmental conditions (12 hr light [8 am]/12 hr dark [8 pm]cycle; room temperature 22±1° C.). Food and tap water were allowed ad libitum. Animals were habituated to the animal facilities at least for a week prior to experiments. The experiments were all carried out during daylight hours.

Animals were fasted overnight before administration of compounds. Animals were administered with compounds (1 mg/kg) by intra-tracheal instillation (2 ml/kg in milliQ water) using an Introcan® Certo cannula after intra-peritoneal (ip) anaesthesia with a mixture of ketamine (150 mg/kg)+medetomidine (1 mg/kg)+butorphanol (1 mg/kg). After administration the animals were given atipamezole (1 mg/kg) to reverse the sedative and analgesic effects induced by the anaesthesia.

Fifteen minutes before sacrifice, animals were anesthetized with pentobarbital 60 mg/kg administrated intra-peritoneally. The brain (without cerebellum) and a fragment of liver were collected into plastic vials containing membrane buffer (3 mM MgCl₂, 1 mM EDTA, 50 mM Tris HCl, pH 7.4). Glass beads (2.5 mm BioSpec Products, Bartlesville, Okla., USA) were added to the vials containing the brain and liver tissues. Tissues were stored at −20° C. until analysis.

### 2. FAAH Activity Determination

2.1. Reagents and Solutions

Anandamide [ethanolamine-1-³H-] was obtained from American Radiochemicals—with a specific activity of 60 Ci/mmol. All other reagents were obtained from Sigma-Aldrich. Optiphase Supermix was obtained from Perkin Elmer.

2.2. Tissue Preparation

Brain and liver tissues were thawed and homogenized in 10 volumes of membrane buffer (3 mM MgCl₂, 1 mM EDTA, 50 mM Tris HCl, pH 7.4) with homogenizer Precellys 24 Dual Tissue Homogenizer (Bertin Technologies) for 2 cycles of 5 sec (5000 rpm).

Total protein in the tissue homogenates was determined with the BioRad Protein Assay (BioRad) using a standard curve of BSA (50-250 g/mL). Tissue homogenates were diluted to appropriate concentration for enzymatic determination in assay buffer (1 mM EDTA, 10 mM Tris HCl, pH 7.6).

2.3. Enzymatic Assay

Reaction mix (total volume 200 μL) contained 2 μM AEA (2 μM AEA+5 nM ³H-AEA), 0.1% fatty acid free BSA, 15 μg (brain) or 5 μg (liver) protein, in assay buffer (1 mM EDTA, 10 mM Tris pH 7.6).

The test compound was used at a level of 1 mg/kg protein. After 15 minutes pre-incubation at 37° C. of the protein sample, reaction was started by the addition of the substrate solution (cold AEA+radiolabelled AEA+BSA). Reaction was carried out for 12 minutes for brain and liver tissues. Reaction was terminated by addition of 400 μL chloroform:methanol (1:1, v/v) solution. Reaction samples were vortex twice, left on ice for 5 minutes and then centrifuged in the microfuge for 7 minutes, 7000 rpm. 200 μL of the obtained supernatants were added to 800 L Optiphase Supermix scintillation cocktail previously distributed in 24-well plates.

Counts per minute (cpm) were determined in a Microbeta TriLux scintillation counter. In each assay blank samples (without protein) were prepared.

### 3. CYPs Metabolic Stability Assay

Stability of the test compounds was performed in HLM (human liver microsomes) in the presence and in the absence of NADPH.

The stability was measured using the incubation mixture (100 μl total volume) contained 1 mg/ml total protein, MgCl₂ 5 mM and 50 mM K-phosphate buffer. Samples were incubated in the presence and in the absence of NADPH 1 mM. Reactions were pre-incubated 5 min and the reaction initiated with the compound under test (at a concentration of 5 μM). Samples were incubated for 60 min in a shaking water bath at 37° C. The reaction was stopped by adding 100 μl of acetonitrile. Samples were then centrifuged, filtered and supematant injected in HLPC-MSD. Test compounds were dissolved in DMSO and the final concentration of DMSO in the reaction was below 0.5% (v/v). At T₀ acetonitrile was added before adding the compound. All experiments were performed with samples in duplicate.

### 4. Solubility Assay

A saturated solution was prepared by adding the test compound in excess to 1 mL of purified water (pH=5.6-5.8) followed by stirring of the mixture for 2 h at room temperature. Thereupon, the un-dissolved material was removed by filtration and the amount of test compound in the filtrate was quantified by HPLC using a calibration curve.

### Results

TABLE 1
The following table shows the FAAH activity
in brain and liver samples for the compounds:

| Compound - | FAAH Activity - Brain | FAAH Activity - Liver |
|---|---|---|
| Example No. | itc (% C) | itc (% C) |
| 1 | 79.0 | 3.2 |
| 2 | 86.8 | 5.1 |
| 3 | 96.0 | 5.3 |
| 4 | 98.7 | 7.2 |
| 5 | 104.7 | 5.7 |
| 6 | 82.1 | 1.7 |
| 7 | 87.2 | 3.5 |
| 8 | 97.6 | 5.4 |
| 9 | 107.2 | 3.9 |
| 10 | 113.4 | 3.2 |
| 11 | 84.2 | 4.4 |
| 12 | 108.7 | 3.8 |
| 13 | 82.1 | 2.9 |
| 14 | 89.3 | 6.0 |
| 15 | 91.1 | 3.0 |
| 16 | 96.9 | 4.5 |
| 17 | 91.9 | 4.2 |
| 18 | 93.3 | 3.4 |
| 19 | 99.2 | 9.2 |
| 20 | 86.4 | 15.8 |
| 21 | 92.4 | 8.2 |
| 22 | 113.8 | 10.7 |
| 23 | 82.1 | 11.5 |
| 24 | 92.6 | 5.7 |
| 25 | 100.7 | 9.0 |
| 26 | 124.3 | 12.4 |
| 27 | 84.3 | 13.9 |
| 28 | 91.0 | 3.4 |
| 29 | 97.6 | 10.5 |
| 30 | 107.1 | 13.7 |
| 31* | 87.7 | 13.1 |
| 32* | 96.0 | 12.8 |
| 33 | 102 | 3.4 |
| 34 | 78.7 | 3.3 |
| 35 | 96.6 | 4.3 |
| 36 | 94.4 | 8.6 |
| 37 | 110.2 | 3.4 |
| 38 | 96.5 | 6.0 |
| 39 | 80.2 | 2.6 |

*These compounds were tested at 0.1 mg/kg rather than at 1 mg/kg as with the other compounds.

As can be seen from the table above, the compounds are peripherally selective, i.e. they inhibit FAAH to a greater extent in peripheral tissue compared to central nervous system tissue. The compounds are also relatively potent.

TABLE 2
The following table shows the metabolic
stability of the compounds:

| Compound - | CYP+ | Control |
|---|---|---|
| Example No. | (% remain) | (% remain) |
| 1 | 30.2 | 111.4 |
| 2 | 68.6 | 98.3 |
| 3 | 17.5 | 103.5 |
| 4 | 11.2 | 100 |
| 5 | 77.3 | 100 |
| 6 | 12.0 | 88.7 |
| 7 | 65.0 | 92.6 |
| 8 | 25.2 | 93.2 |
| 9 | 62.8 | 102.5 |
| 10 | 5.8 | 100.0 |
| 11 | 5.9 | 100.0 |
| 12 | 7.9 | 106.2 |
| 13 | 25.1 | 103.5 |
| 14 | 68.6 | 100.9 |
| 15 | 87.1 | 94.4 |
| 16 | 17.0 | 101.4 |
| 17 | 21.4 | 103.4 |
| 18 | 76.8 | 97.6 |
| 24 | 76.2 | 91.5 |
| 25 | 49.1 | 61.5 |
| 26 | 81.4 | 97.7 |
| 27 | 26.7 | 96.9 |
| 28 | 66.0 | 84.8 |
| 29 | 79.5 | 79.6 |
| 30 | 64.8 | 97.8 |
| 31 | 18.5 | 111.7 |
| 32 | 14.2 | 115.2 |
| 33 | 70.4 | 112.2 |
| 34 | 9.1 | 105.4 |
| 35 | 81.0 | 73.8 |

The above table shows that many of the compounds are metabolised by CYP enzymes. For example, compounds may be metabolised into inactive compounds which can help to ensure the compounds are peripherally selective. Further, compounds may be metabolised into an alternative form which is more peripherally selective.

TABLE 3
The following table shows the aqueous
solubility of selected compounds:

| Compound - | Solubility at room |
|---|---|
| Example No. | temperature (mg/mL) |
| 3 | 7.7 |
| 13 | >16.0 |
| 17 | 14.1 |
| 21 | 4.3 |
| 23 | >16.0 |
| 33 | >14.9 |
| 34 | >14.7 |
| 35 | 10.2 |

The above table shows that the compounds are relatively water soluble. For example, most of the compounds have a solubility of more than 10 mg/ml. The majority of the compounds have a solubility of more than 14 mg/ml. A solubility of between 10 mg/ml and 33 mg/ml is defined as being sparingly soluble by the US Pharmacopeia. However, in the current application, such a solubility is sufficient for the relevant indication (such as treatment of ocular conditions) because other factors also need to be taken into account, such as the potency of the compounds. For example, having a relatively high water solubility may reduce the potency of the compound in terms of FAAH inhibition.

1. A compound having Formula I: 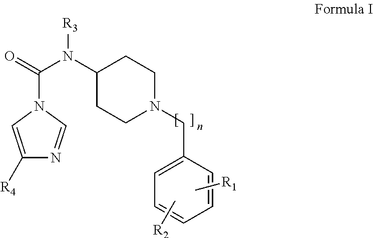
wherein:
R1 is selected from hydrogen, halogen, hydroxyl and C₁₋₄ alkoxy;
R2 is selected from hydrogen, halogen, hydroxyl and C₁₋₄ alkoxy;
R3 is C₁₋₄ alkyl;
R4 is aryl which is substituted with a group selected from OSO₂NH₂, NHCONH₂, NHSO₂NH₂, NHSO₂C₁₋₄ alkyl and CONH₂; and
n is 0 or 1;
or a pharmaceutically acceptable salt thereof;
provided that the compound is not N-(1-benzylpiperidin-4-yl)-N-methyl-4-(4-(sulfamoylamino)phenyl)-1H-imidazole-1-carboxamide or N-(1-benzylpiperidin-4-yl)-N-methyl-4-(3-(methylsulfonamido)phenyl)-1H-imidazole-1-carboxamide.

2. The compound of claim 1, wherein R1 is selected from hydroxyl and C₁₋₄ alkoxy.

3. The compound of claim 1 or claim 2, wherein R1 is selected from hydroxyl and methoxy.

4. The compound of any preceding claim, wherein R1 is hydroxyl.

5. The compound of any preceding claim, wherein R2 is selected from hydrogen, fluorine, hydroxyl and methoxy.

6. The compound of any preceding claim, wherein R1 is hydroxyl and R2 is selected from hydrogen, fluorine and hydroxyl.

7. The compound of any preceding claim, wherein R3 is methyl.

8. The compound of any preceding claim, wherein R4 is aryl which is substituted with a group selected from OSO₂NH₂, NHCONH₂, NHSO₂NH₂, NHSO₂CH₃ and CONH₂.

9. The compound of any preceding claim, wherein R4 is phenyl which is substituted with a group selected from OSO₂NH₂, NHCONH₂, NHSO₂NH₂, NHSO₂CH₃ and CONH₂.

10. The compound of any preceding claim, wherein R4 is phenyl which is substituted with OSO₂NH₂.

11. The compound of claim 10, wherein the OSO₂NH₂ group is at the meta or para position.

12. The compound of any one of claims 1 to 9, wherein R4 is phenyl which is substituted with NHCONH₂.

13. The compound of claim 12, wherein the NHCONH₂ group is at the meta position.

14. The compound of any one of claims 1 to 9, wherein R4 is phenyl which is substituted with NHSO₂NH₂.

15. The compound of claim 14, wherein the NHSO₂NH₂ group is at the meta position.

16. The compound of any one of claims 1 to 9, wherein R4 is phenyl which is substituted with NHSO₂CH₃.

17. The compound of claim 16, wherein the NHSO₂CH₃ group is at the meta position.

18. The compound of any one of claims 1 to 9, wherein R4 is phenyl which is substituted with CONH₂.

19. The compound of claim 18, wherein the CONH₂ group is at the meta or at the para position.

20. The compound of any preceding claim, wherein when n is 1.

21. The compound of claim 1, having Formula VI: 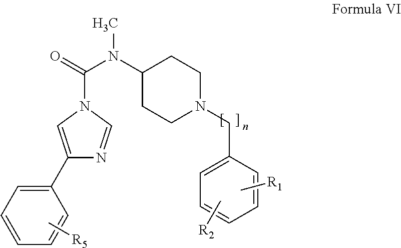
wherein:
R1 is selected from hydroxyl and methoxy;
R2 is selected from hydrogen, halogen, hydroxyl and methoxy;
R5 is selected from OSO₂NH₂, NHCONH₂, NHSO₂NH₂, NHSO₂CH₃ and CONH₂; and
n is 0 or 1;
or a pharmaceutically acceptable salt thereof.

22. A pharmaceutical composition comprising a compound according to any one of claims 1 to 21, together with one or more pharmaceutically acceptable excipients.

23. The pharmaceutical composition of claim 22, further comprising one or more additional active pharmaceutical ingredients such as anandamide, N-oleoylethanolamine or N-palmitoylethanolamine.

24. The pharmaceutical composition of claim 22 or claim 23, wherein the composition is for topical administration.

25. A compound according to any one of claims 1 to 21 or a composition according to any one of claims 22 to 24 for use in therapy.

26. A compound according to any one of claims 1 to 21 or a composition according to any one of claims 22 to 24 for use in the treatment or prevention of a condition whose development or symptoms are linked to a substrate of the FAAH enzyme.

27. A method of treatment or prevention of a condition whose development or symptoms are linked to a substrate of the FAAH enzyme, the method comprising the administration, to a subject in need of such treatment or prevention, of a therapeutically effective amount of a compound according to any one of claims 1 to 21 or a composition according to any one of claims 22 to 24.

28. The method of claim 27, wherein the compound or the composition is administered topically.

29. A compound for use according to claim 26 or a method according to claim 27, wherein the condition is a disorder associated with the endocannabinoid system.

30. A compound or a method according to claim 29, wherein the condition is an ocular condition.

31. A compound or a method according to claim 29, wherein the disorder is selected from ocular hypertension, retinopathy, glaucoma, ocular pain, chronic corneal pain, dry eye syndrome, post-surgical recovery, ocular inflammatory disorders such as uveitis, scleritis, episcleritis, episclera, keratitis, retinal vasculitis and chronic conjunctivitis, reduction of L-dopa-induced hyperactivity in adjunctive dopamine replacement therapy, bladder control and stress-related neuroinflammatory disorders such as post-traumatic stress disorder, multiple sclerosis and stroke.
